SEQUENCE LISTING (2760 sequences)

SEQ ID NO: 1 - protein, 736 aa, unidentified
  source 1..736: note = Dependovirus adeno-associated virus 1
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEEVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDEDKFFPMSGVMIFGKESAGASNTALDNVMITDEEEIKATNPVATERFGTVAVNFQSSSTDPATGDVHAMGALPGMVWQDRDVYLQGPIWAKIPHTDGHFHPSPLMGGFGLKNPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 2 - protein, 735 aa, unidentified [sequence omitted]

SEQ ID NO: 3 - protein, 736 aa, unidentified
  source 1..736: note = Dependovirus adeno-associated virus 3
MAADGYLPDWLEDNLSEGIREWWALKPGVPQPKANQQHQDNRRGLVLPGYKYLGPGNGLDKGEPVNEADAAALEHDKAYDQQLKAGDNPYLKYNHADAEFQERLQEDTSFGGNLGRAVFQAKKRILEPLGLVEEAAKTAPGKKRPVDQSPQEPDSSSGVGKSGKQPARKRLNFGQTGDSESVPDPQPLGEPPAAPTSLGSNTMASGGGAPMADNNEGADGVGNSSGNWHCDSQWLGDRVITTSTRTWALPTYNNHLYKQISSQSGASNDNHYFGYSTPWGYFDFNRFHCHFSPRDWQRLINNNWGFRPKKLSFKLFNIQVKEVTQNDGTTTIANNLTSTVQVFTDSEYQLPYVLGSAHQGCLPPFPADVFMVPQYGYLTLNNGSQAVGRSSFYCLEYFPSQMLRTGNNFQFSYTFEDVPFHSSYAHSQSLDRLMNPLIDQYLYYLNRTQGTTSGTTNQSRLLFSQAGPQSMSLQARNWLPGPCYRQQRLSKTANDNNNSNFPWTAASKYHLNGRDSLVNPGPAMASHKDDEEKFFPMHGNLIFGKEGTTASNAELDNVMITDEEEIRTTNPVATEQYGTVANNLQSSNTAPTTRTVNDQGALPGMVWQDRDVYLQGPIWAKIPHTDGHFHPSPLMGGFGLKHPPPQIMIKNTPVPANPPTTFSPAKFASFITQYSTGQVSVEIEWELQKENSKRWNPEIQYTSNYNKSVNVDFTVDTNGVYSEPRPIGTRYLTRNL

SEQ ID NO: 4 - protein, 734 aa, unidentified
  source 1..734: note = Dependovirus adeno-associated virus 4
MTDGYLPDWLEDNLSEGVREWWALQPGAPKPKANQQHQDNARGLVLPGYKYLGPGNGLDKGEPVNAADAAALEHDKAYDQQLKAGDNPYLKYNHADAEFQQRLQGDTSFGGNLGRAVFQAKKRVLEPLGLVEQAGETAPGKKRPLIESPQQPDSSTGIGKKGKQPAKKKLVFEDETGAGDGPPEGSTSGAMSDDSEMRAAAGGAAVEGGQGADGVGNASGDWHCDSTWSEGHVTTTSTRTWVLPTYNNHLYKRLGESLQSNTYNGFSTPWGYFDFNRFHCHFSPRDWQRLINNNWGMRPKAMRVKIFNIQVKEVTTSNGETTVANNLTSTVQIFADSSYELPYVMDAGQEGSLPPFPNDVFMVPQYGYCGLVTGNTSQQQTDRNAFYCLEYFPSQMLRTGNNFEITYSFEKVPFHSMYAHSQSLDRLMNPLIDQYLWGLQSTTTGTTLNAGTATTNFTKLRPTNFSNFKKNWLPGPSIKQQGFSKTANQNYKIPATGSDSLIKYETHSTLDGRWSALTPGPPMATAGPADSKFSNSQLIFAGPKQNGNTATVPGTLIFTSEEELAATNATDTDMWGNLPGGDQSNSNLPTVDRLTALGAVPGMVWQNRDIYYQGPIWAKIPHTDGHFHPSPLIGGFGLKHPPPQIFIKNTPVPANPATTFSSTPVNSFITQYSTGQVSVQIDWEIQKERSKRWNPEVQFTSNYGQQNSLLWAPDAAGKYTEPRAIGTRYLTHHL

SEQ ID NO: 5 - protein, 724 aa, unidentified
  source 1..724: note = Dependovirus adeno-associated virus 5
MSFVDHPPDWLEEVGEGLREFLGLEAGPPKPKPNQQHQDQARGLVLPGYNYLGPGNGLDRGEPVNRADEVAREHDISYNEQLEAGDNPYLKYNHADAEFQEKLADDTSFGGNLGKAVFQAKKRVLEPFGLVEEGAKTAPTGKRIDDHFPKRKKARTEEDSKPSTSSDAEAGPSGSQQLQIPAQPASSLGADTMSAGGGGPLGDNNQGADGVGNASGDWHCDSTWMGDRVVTKSTRTWVLPSYNNHQYREIKSGSVDGSNANAYFGYSTPWGYFDFNRFHSHWSPRDWQRLINNYWGFRPRSLRVKIFNIQVKEVTVQDSTTTIANNLTSTVQVFTDDDYQLPYVVGNGTEGCLPAFPPQVFTLPQYGYATLNRDNTENPTERSSFFCLEYFPSKMLRTGNNFEFTYNFEEVPFHSSFAPSQNLFKLANPLVDQYLYRFVSTNNTGGVQFNKNLAGRYANTYKNWFPGPMGRTQGWNLGSGVNRASVSAFATTNRMELEGASYQVPPQPNGMTNNLQGSNTYALENTMIFNSQPANPGTTATYLEGNMLITSESETQPVNRVAYNVGGQMATNNQSSTTAPATGTYNLQEIVPGSVWMERDVYLQGPIWAKIPETGAHFHPSPAMGGFGLKHPPPMMLIKNTPVPGNITSFSDVPVSSFITQYSTGQVTVEMEWELKKENSKRWNPEIQYTNNYNDPQFVDFAPDSTGEYRTTRPIGTRYLTRPL

SEQ ID NO: 6 - protein, 736 aa, unidentified
  source 1..736: note = Dependovirus adeno-associated virus 6
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEQSPQEPDSSSGIGKTGQQPAKKRLNFGQTGDSESVPDPQPLGEPPATPAAVGPTTMASGGGAPMADNNEGADGVGNASGNWHCDSTWLGDRVITTSTRTWALPTYNNHLYKQISSASTGASNDNHYFGYSTPWGYFDFNRFHCHFSPRDWQRLINNNWGFRPKRLNFKLFNIQVKEVTTNDGVTTIANNLTSTVQVFSDSEYQLPYVLGSAHQGCLPPFPADVFMIPQYGYLTLNNGSQAVGRSSFYCLEYFPSQMLRTGNNFTFSYTFEDVPFHSSYAHSQSLDRLMNPLIDQYLYYLNRTQNQSGSAQNKDLLFSRGSPAGMSVQPKNWLPGPCYRQQRVSKTKTDNNNSNFTWTGASKYNLNGRESIINPGTAMASHKDDKDKFFPMSGVMIFGKESAGASNTALDNVMITDEEEIKATNPVATERFGTVAVNLQSSSTDPATGDVHVMGALPGMVWQDRDVYLQGPIWAKIPHTDGHFHPSPLMGGFGLKHPPPQILIKNTPVPANPPAEFSATKFASFITQYSTGQVSVEIEWELQKENSKRWNPEVQYTSNYAKSANVDFTVDNNGLYTEPRPIGTRYLTRPL

SEQ ID NO: 7 - protein, 737 aa, unidentified
  source 1..737: note = Dependovirus adeno-associated virus 7
MAADGYLPDWLEDNLSEGIREWWDLKPGAPKPKANQQKQDNGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPAKKRPVEPSPQRSPDSSTGIGKKGQQPARKRLNFGQTGDSESVPDPQPLGEPPAAPSSVGSGTVAAGGGAPMADNNEGADGVGNASGNWHCDSTWLGDRVITTSTRTWALPTYNNHLYKQISSETAGSTNDNTYFGYSTPWGYFDFNRFHCHFSPRDWQRLINNNWGFRPKKLRFKLFNIQVKEVTTNDGVTTIANNLTSTIQVFSDSEYQLPYVLGSAHQGCLPPFPADVFMIPQYGYLTLNNGSQSVGRSSFYCLEYFPSQMLRTGNNFEFSYSFEDVPFHSSYAHSQSLDRLMNPLIDQYLYYLARTQSNPGGTAGNRELQFYQGGPSTMAEQAKNWLPGPCFRQQRVSKTLDQNNNSNFAWTGATKYHLNGRNSLVNPGVAMATHKDDEDRFFPSSGVLIFGKTGATNKTTLENVLMTNEEEIRPTNPVATEEYGIVSSNLQAANTAAQTQVVNNQGALPGMVWQNRDVYLQGPIWAKIPHTDGNFHPSPLMGGFGLKHPPPQILIKNTPVPANPPEVFTPAKFASFITQYSTGQVSVEIEWELQKENSKRWNPEIQYTSNFEKQTGVDFAVDSQGVYSEPRPIGTRYLTRNL

SEQ ID NO: 8 - protein, 738 aa, unidentified
  source 1..738: note = Dependovirus adeno-associated virus 8
MAADGYLPDWLEDNLSEGIREWWALKPGAPKPKANQQKQDDGRGLVLPGYKYLGPFNGLDKGEPVNAADAAALEHDKAYDQQLQAGDNPYLRYNHADAEFQERLQEDTSFGGNLGRAVFQAKKRVLEPLGLVEEGAKTAPGKKRPVEPSPQRSPDSSTGIGKKGQQPARKRLNFGQTGDSESVPDPQPLGEPPAAPSGVGPNTMAAGGGAPMADNNEGADGVGSSSGNWHCDSTWLGDRVITTSTRTWALPTYNNHLYKQISNGTSGGATNDNTYFGYSTPWGYFDFNRFHCHFSPRDWQRLINNNWGFRPKRLSFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFQFTYTFEDVPFHSSYAHSQSLDRLMNPLIDQYLYYLSRTQTTGGTANTQTLGFSQGGPNTMANQAKNWLPGPCYRQQRVSTTTGQNNNSNFAWTAGTKYHLNGRNSLANPGIAMATHKDDEERFFPSNGILIFGKQNAARDNADYSDVMLTSEEEIKTTNPVATEEYGIVADNLQQQNTAPQIGTVNSQGALPGMVWQNRDVYLQGPIWAKIPHTDGNFHPSPLMGGFGLKHPPPQILIKNTPVPADPPTTFNQSKLNSFITQYSTGQVSVEIEWELQKENSKRWNPEIQYTSNYYKSTSVDFAVNTEGVYSEPRPIGTRYLTRNL

SEQ ID NO: 9 - protein, 736 aa, unidentified
  source 1..736: note = Dependovirus adeno-associated virus 9
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 10 - protein, 738 aa, unidentified
  source 1..738: note = Dependovirus adeno-associated virus rh.10
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPVEPSPQRSPDSSTGIGKKGQQPAKKRLNFGQTGDSESVPDPQPIGEPPAGPSGLGSGTMAAGGGAPMADNNEGADGVGSSSGNWHCDSTWLGDRVITTSTRTWALPTYNNHLYKQISNGTSGGSTNDNTYFGYSTPWGYFDFNRFHCHFSPRDWQRLINNNWGFRPKRLNFKLFNIQVKEVTQNEGTKTIANNLTSTIQVFTDSEYQLPYVLGSAHQGCLPPFPADVFMIPQYGYLTLNNGSQAVGRSSFYCLEYFPSQMLRTGNNFEFSYQFEDVPFHSSYAHSQSLDRLMNPLIDQYLYYLSRTQSTGGTAGTQQLLFSQAGPNNMSAQAKNWLPGPCYRQQRVSTTLSQNNNSNFAWTGATKYHLNGRDSLVNPGVAMATHKDDEERFFPSSGVLMFGKQGAGKDNVDYSSVMLTSEEEIKTTNPVATEQYGVVADNLQQQNAAPIVGAVNSQGALPGMVWQNRDVYLQGPIWAKIPHTDGNFHPSPLMGGFGLKHPPPQILIKNTPVPADPPTTFSQAKLASFITQYSTGQVSVEIEWELQKENSKRWNPEIQYTSNYYKSTNVDFAVNTDGTYSEPRPIGTRYLTRNL

SEQ ID NO: 11 - protein, 733 aa, unidentified
  source 1..733: note = Dependovirus adeno-associated virus 11
MAADGYLPDWLEDNLSEGIREWWDLKPGAPKPKANQQKQDDGRGLVLPGYKYLGPFNGLDKGEPVNAADAAALEHDKAYDQQLKAGDNPYLRYNHADAEFQERLQEDTSFGGNLGRAVFQAKKRVLEPLGLVEEGAKTAPGKKRPLESPQEPDSSSGIGKKGKQPARKRLNFEEDTGAGDGPPEGSDTSAMSSDIEMRAAPGGNAVDAGQGSDGVGNASGDWHCDSTWSEGKVTTTSTRTWVLPTYNNHLYLRLGTTSSSNTYNGFSTPWGYFDFNRFHCHFSPRDWQRLINNNWGLRPKAMRVKIFNIQVKEVTTSNGETTVANNLTSTVQIFADSSYELPYVMDAGQEGSLPPFPNDVFMVPQYGYCGIVTGENQNQTDRNAFYCLEYFPSQMLRTGNNFEMAYNFEKVPFHSMYAHSQSLDRLMNPLLDQYLWHLQSTTSGETLNQGNAATTFGKIRSGDFAFYRKNWLPGPCVKQQRFSKTASQNYKIPASGGNALLKYDTHYTLNNRWSNIAPGPPMATAGPSDGDFSNAQLIFPGPSVTGNTTTSANNLLFTSEEEIAATNPRDTDMFGQIADNNQNATTAPITGNVTAMGVLPGMVWQNRDIYYQGPIWAKIPHADGHFHPSPLIGGFGLKHPPPQIFIKNTPVPANPATTFTAARVDSFITQYSTGQVAVQIEWEIEKERSKRWNPEVQFTSNYGNQSSMLWAPDTTGKYTEPRVIGSRYLTNHL

SEQ ID NO: 12 - protein, 8 aa, synthetic construct
  REGION 1..8: note = Amino acid substitution
SCQPTVMN

SEQ ID NO: 13 - protein, 8 aa, synthetic construct
  REGION 1..8: note = Amino acid substitution
FGVPNQPL

SEQ ID NO: 14 - protein, 8 aa, synthetic construct
  REGION 1..8: note = Amino acid substitution
QRGQAAPF

SEQ ID NO: 15 - protein, 8 aa, synthetic construct
  REGION 1..8: note = Amino acid substitution
GDYAPIRE

SEQ ID NO: 16 - protein, 8 aa, synthetic construct
  REGION 1..8: note = Amino acid substitution
KTRRIVQH

SEQ ID NO: 17 - protein, 8 aa, synthetic construct
  REGION 1..8: note = Amino acid substitution
FGFPNQPL

SEQ ID NO: 18 - protein, 8 aa, synthetic construct
  REGION 1..8: note = Amino acid substitution
RQDQPINA

SEQ ID NO: 19 - protein, 8 aa, synthetic construct
  REGION 1..8: note = Amino acid substitution
SKVESWTE

SEQ ID NO: 20 - protein, 8 aa, synthetic construct
  REGION 1..8: note = Amino acid substitution
STVDSIAI

SEQ ID NO: 21 - skipped

SEQ ID NO: 22 - protein, 5 aa, synthetic construct
  REGION 1..5: note = peptide motif
FVFLP

SEQ ID NO: 23 - skipped

SEQ ID NO: 24 - protein, 4 aa, synthetic construct
  REGION 1..4: note = peptide motif
RGNR

SEQ ID NO: 25 - protein, 7 aa, synthetic construct
  REGION 1..7: note = targeting sequence
  VARIANT 7: note = X can be G or S
NSVRDLX

SEQ ID NO: 26 - protein, 7 aa, synthetic construct
  REGION 1..7: note = targeting sequence
PRSVTVP

SEQ ID NO: 27 - protein, 7 aa, synthetic construct
  REGION 1..7: note = targeting sequence
  VARIANT 6: note = X can be any amino acid
  VARIANT 7: note = X can be S or A
NSVSSXX

SEQ ID NO: 28 - protein, 6 aa, synthetic construct
  REGION 1..6: note = targeting sequence
NGRAHA

SEQ ID NO: 29 - protein, 7 aa, synthetic construct
  REGION 1..7: note = targeting sequence
QPEHSST

SEQ ID NO: 30 - protein, 7 aa, synthetic construct
  REGION 1..7: note = targeting sequence
VNTANST

SEQ ID NO: 31 - protein, 6 aa, synthetic construct
  REGION 1..6: note = targeting sequence
HGPMQS

SEQ ID NO: 32 - protein, 7 aa, synthetic construct
  REGION 1..7: note = targeting sequence
PHKPPLA

SEQ ID NO: 33 - protein, 7 aa, synthetic construct
  REGION 1..7: note = targeting sequence
IKNNEMW

SEQ ID NO: 34 - protein, 7 aa, synthetic construct
  REGION 1..7: note = targeting sequence
RNLDTPM

SEQ ID NO: 35 - protein, 7 aa, synthetic construct
  REGION 1..7: note = targeting sequence
VDSHRQS

SEQ ID NO: 36 - protein, 7 aa, synthetic construct
  REGION 1..7: note = targeting sequence
YDSKTKT

SEQ ID NO: 37 - protein, 7 aa, synthetic construct
  REGION 1..7: note = targeting sequence
SQLPHQK

SEQ ID NO: 38 - protein, 7 aa, synthetic construct
  REGION 1..7: note = targeting sequence
STMQQNT

SEQ ID NO: 39 - protein, 7 aa, synthetic construct
  REGION 1..7: note = targeting sequence
TERYMTQ

SEQ ID NO: 40 - protein, 7 aa, synthetic construct
  REGION 1..7: note = targeting sequence
QPEHSST

SEQ ID NO: 41 - protein, 7 aa, synthetic construct
  REGION 1..7: note = targeting sequence
DASLSTS

SEQ ID NO: 42 - protein, 6 aa, synthetic construct
  REGION 1..6: note = targeting sequence
DLPNKT

SEQ ID NO: 43 - protein, 7 aa, synthetic construct
  REGION 1..7: note = targeting sequence
DLTAARL

SEQ ID NO: 44 - protein, 7 aa, synthetic construct
  REGION 1..7: note = targeting sequence
EPHQFNY

SEQ ID NO: 45 - protein, 7 aa, synthetic construct
  REGION 1..7: note = targeting sequence
EPQSNHT

SEQ ID NO: 46 - protein, 7 aa, synthetic construct
  REGION 1..7: note = targeting sequence
MSSWPSQ

SEQ ID NO: 47 - protein, 7 aa, synthetic construct
  REGION 1..7: note = targeting sequence
NPKHNAT

SEQ ID NO: 48 - protein, 7 aa, synthetic construct
  REGION 1..7: note = targeting sequence
PDGMRTT

SEQ ID NO: 49 - protein, 7 aa, synthetic construct
  REGION 1..7: note = targeting sequence
PNNNKTT

SEQ ID NO: 50 - protein, 7 aa, synthetic construct
  REGION 1..7: note = targeting sequence
QSTTHDS

SEQ ID NO: 51 - protein, 7 aa, synthetic construct
  REGION 1..7: note = targeting sequence
TGSKQKQ

SEQ ID NO: 52 - protein, 7 aa, synthetic construct
  REGION 1..7: note = targeting sequence
SLKHQAL

SEQ ID NO: 53 - protein, 7 aa, synthetic construct
  REGION 1..7: note = targeting sequence
SPIDGEQ

SEQ ID NO: 54 - protein, 8 aa, synthetic construct
  REGION 1..8: note = targeting sequence
WIFPWIQL

SEQ ID NO: 55 - protein, 9 aa, synthetic construct
  REGION 1..9: note = targeting sequence
CDCRGDCFC

SEQ ID NO: 56 - protein, 5 aa, synthetic construct
  REGION 1..5: note = targeting sequence
CNGRC

SEQ ID NO: 57 - protein, 7 aa, synthetic construct
  REGION 1..7: note = targeting sequence
CPRECES

SEQ ID NO: 58 - protein, 10 aa, synthetic construct
  REGION 1..10: note = targeting sequence
CTTHWGFTLC

SEQ ID NO: 59 - protein, 9 aa, synthetic construct
  REGION 1..9: note = targeting sequence
CGRRAGGSC

SEQ ID NO: 60 - protein, 9 aa, synthetic construct
  REGION 1..9: note = targeting sequence
CKGGRAKDC

SEQ ID NO: 61 - protein, 9 aa, synthetic construct
  REGION 1..9: note = targeting sequence
CVPELGHEC

SEQ ID NO: 62 - protein, 9 aa, synthetic construct
  REGION 1..9: note = targeting sequence
CRRETAWAK

SEQ ID NO: 63 - protein, 14 aa, synthetic construct
  REGION 1..14: note = targeting sequence
VSWFSHRYSPFAVS

SEQ ID NO: 64 - protein, 13 aa, synthetic construct
  REGION 1..13: note = targeting sequence
GYRDGYAGPILYN

SEQ ID NO: 65-66 - skipped (2 entries)

SEQ ID NO: 67 - protein, 7 aa, synthetic construct
  REGION 1..7: note = targeting sequence
RPLPPLP

SEQ ID NO: 68 - protein, 7 aa, synthetic construct
  REGION 1..7: note = targeting sequence
APPLPPR

SEQ ID NO: 69 - protein, 12 aa, synthetic construct
  REGION 1..12: note = targeting sequence
DVFYPYPYASGS

SEQ ID NO: 70 - protein, 6 aa, synthetic construct
  REGION 1..6: note = targeting sequence
MYWYPY

SEQ ID NO: 71 - protein, 12 aa, synthetic construct
  REGION 1..12: note = targeting sequence
DITWDQLWDLMK

SEQ ID NO: 72 - protein, 8 aa, synthetic construct
  REGION 1..8: note = targeting sequence
  VARIANT 5: note = X can be G or L
CWDDXWLC

SEQ ID NO: 73 - protein, 14 aa, synthetic construct
  REGION 1..14: note = targeting sequence
EWCEYLGGYLRCYA

SEQ ID NO: 74 - protein, 14 aa, synthetic construct
  REGION 1..14: note = targeting sequence
  VARIANT 2: note = X can be any amino acid
  VARIANT 4..5: note = X can be any amino acid
  VARIANT 8: note = X can be any amino acid
  VARIANT 11: note = X can be any amino acid
  VARIANT 13: note = X can be any amino acid
YXCXXGPXTWXCXP

SEQ ID NO: 75 - protein, 14 aa, synthetic construct
  REGION 1..14: note = targeting sequence
IEGPTLRQWLAARA

SEQ ID NO: 76 - skipped

SEQ ID NO: 77 - protein, 7 aa, synthetic construct
  REGION 1..7: note = targeting sequence
  VARIANT 1: note = X can be any amino acid
  VARIANT 3..4: note = X can be any amino acid
XFXXYLW

SEQ ID NO: 78 - protein, 6 aa, synthetic construct
  REGION 1..6: note = targeting sequence
RWGLCD

SEQ ID NO: 79 - protein, 13 aa, synthetic construct
  REGION 1..13: note = targeting sequence
MSRPACPPNDKYE

SEQ ID NO: 80 - protein, 8 aa, synthetic construct
  REGION 1..8: note = targeting sequence
CLRSGRGC

SEQ ID NO: 81 - protein, 9 aa, synthetic construct
  REGION 1..9: note = targeting sequence
CHWMFSPWC

SEQ ID NO: 82 - skipped

SEQ ID NO: 83 - protein, 8 aa, synthetic construct
  REGION 1..8: note = targeting sequence
CSSRLDAC

SEQ ID NO: 84 - protein, 7 aa, synthetic construct
  REGION 1..7: note = targeting sequence
CLPVASC

SEQ ID NO: 85 - protein, 13 aa, synthetic construct
  REGION 1..13: note = targeting sequence
CGFECVRQCPERC

SEQ ID NO: 86 - protein, 13 aa, synthetic construct
  REGION 1..13: note = targeting sequence
CVALCREACGEGC

SEQ ID NO: 87 - protein, 9 aa, synthetic construct
  REGION 1..9: note = targeting sequence
SWCEPGWCR

SEQ ID NO: 88 - protein, 6 aa, synthetic construct
  REGION 1..6: note = targeting sequence
YSGWGW

SEQ ID NO: 89 - protein, 7 aa, synthetic construct
  REGION 1..7: note = targeting sequence
GLSGGRS

SEQ ID NO: 90 - protein, 7 aa, synthetic construct
  REGION 1..7: note = targeting sequence
LMLPRAD

SEQ ID NO: 91 - protein, 9 aa, synthetic construct
  REGION 1..9: note = targeting sequence
CSCFRDVCC

SEQ ID NO: 92 - protein, 9 aa, synthetic construct
  REGION 1..9: note = targeting sequence
CRDVVSVIC

SEQ ID NO: 93 - protein, 5 aa, synthetic construct
  REGION 1..5: note = targeting sequence
CNGRC

SEQ ID NO: 94 - protein, 6 aa, synthetic construct
  REGION 1..6: note = targeting sequence
MARSGL

SEQ ID NO: 95 - protein, 6 aa, synthetic construct
  REGION 1..6: note = targeting sequence
MARAKE

SEQ ID NO: 96 - protein, 6 aa, synthetic construct
  REGION 1..6: note = targeting sequence
MSRTMS

SEQ ID NO: 97 - protein, 6 aa, synthetic construct
  REGION 1..6: note = targeting sequence
KCCYSL

SEQ ID NO: 98 - protein, 14 aa, synthetic construct
  REGION 1..14: note = targeting sequence
MYWGDSHWLQYWYE

SEQ ID NO: 99 - protein, 7 aa, synthetic construct
  REGION 1..7: note = targeting sequence
MQLPLAT

SEQ ID NO: 100 - protein, 4 aa, synthetic construct
  REGION 1..4: note = targeting sequence
EWLS

SEQ ID NO: 101 - protein, 4 aa, synthetic construct
  REGION 1..4: note = targeting sequence
SNEW

SEQ ID NO: 102 - protein, 4 aa, synthetic construct
  REGION 1..4: note = targeting sequence
TNYL

SEQ ID NO: 103 - protein, 8 aa, synthetic construct
  REGION 1..8: note = targeting sequence
WIFPWIQL

SEQ ID NO: 104 - protein, 12 aa, synthetic construct
  REGION 1..12: note = targeting sequence
WDLAWMFRLPVG

SEQ ID NO: 105 - protein, 13 aa, synthetic construct
  REGION 1..13: note = targeting sequence
CTVALPGGYVRVC

SEQ ID NO: 106 - protein, 9 aa, synthetic construct
  REGION 1..9: note = targeting sequence
CVPELGHEC

SEQ ID NO: 107 - protein, 9 aa, synthetic construct
  REGION 1..9: note = targeting sequence
CGRRAGGSC

SEQ ID NO: 108 - protein, 13 aa, synthetic construct
  REGION 1..13: note = targeting sequence
CVAYCIEHHCWTC

SEQ ID NO: 109 - protein, 12 aa, synthetic construct
  REGION 1..12: note = targeting sequence
CVFAHNYDYLVC

SEQ ID NO: 110 - protein, 10 aa, synthetic construct
  REGION 1..10: note = targeting sequence
CVFTSNYAFC

SEQ ID NO: 111 - protein, 7 aa, synthetic construct
  REGION 1..7: note = targeting sequence
VHSPNKK

SEQ ID NO: 112 - protein, 9 aa, synthetic construct
  REGION 1..9: note = targeting sequence
CDCRGDCFC

SEQ ID NO: 113 - protein, 7 aa, synthetic construct
  REGION 1..7: note = targeting sequence
CRGDGWC

SEQ ID NO: 114 - protein, 6 aa, synthetic construct
  REGION 1..6: note = targeting sequence
  VARIANT 1: note = X can be any amino acid
  VARIANT 6: note = X can be any amino acid
XRGCDX

SEQ ID NO: 115 - skipped

SEQ ID NO: 116 - protein, 10 aa, synthetic construct
  REGION 1..10: note = targeting sequence
CTTHWGFTLC

SEQ ID NO: 117 - protein, 11 aa, synthetic construct
  REGION 1..11: note = targeting sequence
SGKGPRQITAL

SEQ ID NO: 118 - skipped

SEQ ID NO: 119 - protein, 6 aa, synthetic construct
  REGION 1..6: note = targeting sequence
VYMSPF

SEQ ID NO: 120 - protein, 7 aa, synthetic construct
  REGION 1..7: note = targeting sequence
MQLPLAT

SEQ ID NO: 121 - protein, 7 aa, synthetic construct
  REGION 1..7: note = targeting sequence
ATWLPPR

SEQ ID NO: 122 - protein, 12 aa, synthetic construct
  REGION 1..12: note = targeting sequence
HTMYYHHYQHHL

SEQ ID NO: 123 - protein, 19 aa, synthetic construct
  REGION 1..19: note = targeting sequence
SEVGCRAGPLQWLCEKYFG

SEQ ID NO: 124 - protein, 18 aa, synthetic construct
  REGION 1..18: note = targeting sequence
CGLLPVGRPDRNVWRWLC

SEQ ID NO: 125 - protein, 15 aa, synthetic construct
  REGION 1..15: note = targeting sequence
CKGQCDRFKGLPWEC

SEQ ID NO: 126 - protein, 5 aa, synthetic construct
  REGION 1..5: note = targeting sequence
SGRSA

SEQ ID NO: 127 - protein, 4 aa, synthetic construct
  REGION 1..4: note = targeting sequence
WGFP

SEQ ID NO: 128 - protein, 6 aa, synthetic construct
  REGION 1..6: note = targeting sequence
  VARIANT 3..4: note = X can be any amino acid
LWXXAR

SEQ ID NO: 129 - protein, 7 aa, synthetic construct
  REGION 1..7: note = targeting sequence
  VARIANT 1: note = X can be any amino acid
  VARIANT 3..4: note = X can be any amino acid
XFXXYLW

SEQ ID NO: 130 - protein, 17 aa, synthetic construct
  REGION 1..17: note = targeting sequence
AEPMPHSLNFSQYLWYT

SEQ ID NO: 131 - protein, 6 aa, synthetic construct
  REGION 1..6: note = targeting sequence
  VARIANT 4: note = X can be W or F
WAYXSP

SEQ ID NO: 132 - protein, 7 aa, synthetic construct
  REGION 1..7: note = targeting sequence
IELLQAR

SEQ ID NO: 133 - protein, 12 aa, synthetic construct
  REGION 1..12: note = targeting sequence
DITWDQLWDLMK

SEQ ID NO: 134 - protein, 16 aa, synthetic construct
  REGION 1..16: note = targeting sequence
AYTKCSRQWRTCMTTH

SEQ ID NO: 135 - protein, 15 aa, synthetic construct
  REGION 1..15: note = targeting sequence
PQNSKIPGPTFLDPH

SEQ ID NO: 136 - protein, 15 aa, synthetic construct
  REGION 1..15: note = targeting sequence
SMEPALPDWWWKMFK

SEQ ID NO: 137 - protein, 16 aa, synthetic construct
  REGION 1..16: note = targeting sequence
ANTPCGPYTHDCPVKR

SEQ ID NO: 138 - protein, 12 aa, synthetic construct
  REGION 1..12: note = targeting sequence
TACHQHVRMVRP

SEQ ID NO: 139 - protein, 12 aa, synthetic construct
  REGION 1..12: note = targeting sequence
VPWMEPAYQRFL

SEQ ID NO: 140 - protein, 8 aa, synthetic construct
  REGION 1..8: note = targeting sequence
DPRATPGS

SEQ ID NO: 141 - protein, 12 aa, synthetic construct
  REGION 1..12: note = targeting sequence
FRPNRAQDYNTN

SEQ ID NO: 142 - protein, 9 aa, synthetic construct
  REGION 1..9: note = targeting sequence
CTKNSYLMC

SEQ ID NO: 143 - protein, 11 aa, synthetic construct
  REGION 1..11: note = targeting sequence
  VARIANT 2: note = X can be R or Q
  VARIANT 3: note = X can be L or R
  VARIANT 5: note = X can be G or N
  VARIANT 6..7: note = X can be any amino acid
  VARIANT 9: note = X can be A or V
CXXTXXXGXGC

SEQ ID NO: 144 - protein, 9 aa, synthetic construct
  REGION 1..9: note = targeting sequence
CPIEDRPMC

SEQ ID NO: 145 - protein, 12 aa, synthetic construct
  REGION 1..12: note = targeting sequence
HEWSYLAPYPWF

SEQ ID NO: 146 - protein, 9 aa, synthetic construct
  REGION 1..9: note = targeting sequence
MCPKHPLGC

SEQ ID NO: 147 - protein, 15 aa, synthetic construct
  REGION 1..15: note = targeting sequence
RMWPSSTVNLSAGRR

SEQ ID NO: 148 - protein, 20 aa, synthetic construct
  REGION 1..20: note = targeting sequence
SAKTAVSQRVWLPSHRGGEP

SEQ ID NO: 149 - protein, 20 aa, synthetic construct
  REGION 1..20: note = targeting sequence
KSREHVNNSACPSKRITAAL

SEQ ID NO: 150 - protein, 4 aa, synthetic construct
  REGION 1..4: note = targeting sequence
EGFR

SEQ ID NO: 151 - protein, 6 aa, synthetic construct
  REGION 1..6: note = targeting sequence
AGLGVR

SEQ ID NO: 152 - protein, 15 aa, synthetic construct
  REGION 1..15: note = targeting sequence
GTRQGHTMRLGVSDG

SEQ ID NO: 153 - protein, 15 aa, synthetic construct
  REGION 1..15: note = targeting sequence
IAGLATPGWSHWLAL

SEQ ID NO: 154 - protein, 7 aa, synthetic construct
  REGION 1..7: note = targeting sequence
SMSIARL

SEQ ID NO: 155 - protein, 7 aa, synthetic construct
  REGION 1..7: note = targeting sequence
HTFEPGV

SEQ ID NO: 156 - protein, 14 aa, synthetic construct
  REGION 1..14: note = targeting sequence
NTSLKRISNKRRRK

SEQ ID NO: 157 - protein, 15 aa, synthetic construct
  REGION 1..15: note = targeting sequence
LRIKRKRRKRKKTRK

SEQ ID NO: 158-164 - skipped (7 entries)

SEQ ID NO: 165 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTSCQPTVMNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 166 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTFGVPNQPLQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 167 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTQRGQAAPFQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 168 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTGDYAPIREQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 169 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTKTRRIVQHQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 170 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTFGFPNQPLQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 171 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTRQDQPINAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSAQAQAQTGWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 172 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 173 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTINGSGQNQQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 174 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTSCQPTVMNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 175 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTFGVPNQPLQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 176 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTQRGQAAPFQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 177 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTGDYAPIREQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 178 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTKTRRIVQHQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 179 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTFGFPNQPLQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 180 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTRQDQPINAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSKVESWTEWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 181 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTSCQPTVMNQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 182 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTFGVPNQPLQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 183 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTQRGQAAPFQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 184 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTGDYAPIREQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 185 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTKTRRIVQHQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 186 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTFGFPNQPLQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 187 - protein, 736 aa, synthetic construct
  REGION 1..736: note = AAV capsid variant
MAADGYLPDWLEDNLSEGIREWWALKPGAPQPKANQQHQDNARGLVLPGYKYLGPGNGLDKGEPVNAADAAALEHDKAYDQQLKAGDNPYLKYNHADAEFQERLKEDTSFGGNLGRAVFQAKKRLLEPLGLVEEAAKTAPGKKRPVEQSPQEPDSSAGIGKSGAQPAKKRLNFGQTGDTESVPDPQPIGEPPAAPSGVGSLTMASGGGAPVADNNEGADGVGSSSGNWHCDSQWLGDRVITTSTRTWALPTYNNHLYKQISNSTSGGSSNDNAYFGYSTPWGYFDFNRFHCHFSPRDWQRLINNNWGFRPKRLNFKLFNIQVKEVTDNNGVKTIANNLTSTVQVFTDSDYQLPYVLGSAHEGCLPPFPADVFMIPQYGYLTLNDGSQAVGRSSFYCLEYFPSQMLRTGNNFQFSYEFENVPFHSSYAHSQSLDRLMNPLIDQYLYYLSKTRQDQPINAQTLKFSVAGPSNMAVQGRNYIPGPSYRQQRVSTTVTQNNNSEFAWPGASSWALNGRNSLMNPGPAMASHKEGEDRFFPLSGSLIFGKQGTGRDNVDADKVMITNEEEIKTTNPVATESYGQVATNHQSSTVDSIAIWVQNQGILPGMVWQDRDVYLQGPIWAKIPHTDGNFHPSPLMGGFGMKHPPPQILIKNTPVPADPPTAFNKDKLNSFITQYSTGQVSVEIEWELQKENSKRWNPEIQYTSNYYKSNNVEFAVNTEGVYSEPRPIGTRYLTRNL

SEQ ID NO: 188-1373 - skipped (1186 entries)

SEQ ID NO: 1374 - protein, 8 aa, synthetic construct
  REGION 1..8: note = AAV peptide sequence
LSKTQTLK

SEQ ID NO: 1375 - protein, 10 aa, synthetic construct
  REGION 1..10: note = AAV peptide sequence
LSKTDPQTLK

SEQ ID NO: 1376-2590 - skipped (1215 entries)

SEQ ID NO: 2591 - protein, 7 aa, unidentified
  source 1..7: note = Dependovirus adeno-associated virus
SASTGAS

SEQ ID NO: 2592 - protein, 10 aa, unidentified
  source 1..10: note = Dependovirus adeno-associated virus
VFMIPQYGYL

SEQ ID NO: 2593 - protein, 9 aa, unidentified
  source 1..9: note = Dependovirus adeno-associated virus
NQSGSAQNK

SEQ ID NO: 2594 - skipped

SEQ ID NO: 2595 - protein, 8 aa, unidentified
  source 1..8: note = Dependovirus adeno-associated virus
KTDNNNSN

SEQ ID NO: 2596 - protein, 7 aa, unidentified
  source 1..7: note = Dependovirus adeno-associated virus
KDDEDKF

SEQ ID NO: 2597 - protein, 6 aa, unidentified
  source 1..6: note = Dependovirus adeno-associated virus
SAGASN

SEQ ID NO: 2598 - protein, 10 aa, unidentified
  source 1..10: note = Dependovirus adeno-associated virus
STDPATGDVH

SEQ ID NO: 2599 - skipped

SEQ ID NO: 2600 - protein, 7 aa, unidentified
  source 1..7: note = Dependovirus adeno-associated virus
DNNGLYT

SEQ ID NO: 2601 - protein, 6 aa, unidentified
  source 1..6: note = Dependovirus adeno-associated virus
SQSGAS

SEQ ID NO: 2602 - protein, 10 aa, unidentified
  source 1..10: note = Dependovirus adeno-associated virus
VFMVPQYGYL

SEQ ID NO: 2603 - protein, 9 aa, unidentified
  source 1..9: note = Dependovirus adeno-associated virus
TPSGTTTQS

SEQ ID NO: 2604 - skipped

SEQ ID NO: 2605 - protein, 8 aa, unidentified
  source 1..8: note = Dependovirus adeno-associated virus
SADNNNSE

SEQ ID NO: 2606 - protein, 7 aa, unidentified
  source 1..7: note = Dependovirus adeno-associated virus
KDDEEKF

SEQ ID NO: 2607 - protein, 6 aa, unidentified
  source 1..6: note = Dependovirus adeno-associated virus
GSEKTN

SEQ ID NO: 2608 - protein, 10 aa, unidentified
  source 1..10: note = Dependovirus adeno-associated virus
NRQAATADVN

SEQ ID NO: 2609 - skipped

SEQ ID NO: 2610 - protein, 7 aa, unidentified
  source 1..7: note = Dependovirus adeno-associated virus
DTNGVYS

SEQ ID NO: 2611 - protein, 6 aa, unidentified
  source 1..6: note = Dependovirus adeno-associated virus
SQSGAS

SEQ ID NO: 2612 - protein, 10 aa, unidentified
  source 1..10: note = Dependovirus adeno-associated virus
VFMVPQYGYL

SEQ ID NO: 2613 - protein, 9 aa, unidentified
  source 1..9: note = Dependovirus adeno-associated virus
TTSGTTNQS

SEQ ID NO: 2614 - skipped

SEQ ID NO: 2615 - protein, 8 aa, unidentified
  source 1..8: note = Dependovirus adeno-associated virus
ANDNNNSN

SEQ ID NO: 2616 - protein, 7 aa, unidentified
  source 1..7: note = Dependovirus adeno-associated virus
KDDEEKF

SEQ ID NO: 2617 - protein, 6 aa, unidentified
  source 1..6: note = Dependovirus adeno-associated virus
GTTASN

SEQ ID NO: 2618 - protein, 10 aa, unidentified
  source 1..10: note = Dependovirus adeno-associated virus
NTAPTTGTVN

SEQ ID NO: 2619 - skipped

SEQ ID NO: 2620 - protein, 7 aa, unidentified
  source 1..7: note = Dependovirus adeno-associated virus
DTNGVYS

SEQ ID NO: 2621 - protein, 8 aa, unidentified
  source 1..8: note = Dependovirus adeno-associated virus
RLGESLQS

SEQ ID NO: 2622 - protein, 10 aa, unidentified
  source 1..10: note = Dependovirus adeno-associated virus
VFMVPQYGYC

SEQ ID NO: 2623 - protein, 9 aa, unidentified
  source 1..9: note = Dependovirus adeno-associated virus
GTTLNAGTA

SEQ ID NO: 2624 - skipped

SEQ ID NO: 2625 - protein, 12 aa, unidentified
  source 1..12: note = Dependovirus adeno-associated virus
ANQNYKIPATGS

SEQ ID NO: 2626 - protein, 7 aa, unidentified
  source 1..7: note = Dependovirus adeno-associated virus
GPADSKF

SEQ ID NO: 2627 - protein, 6 aa, unidentified
  source 1..6: note = Dependovirus adeno-associated virus
QNGNTA

SEQ ID NO: 2628 - protein, 10 aa, unidentified
  source 1..10: note = Dependovirus adeno-associated virus
SNLPTVDRLT

SEQ ID NO: 2629 - skipped

SEQ ID NO: 2630 - protein, 7 aa, unidentified
  source 1..7: note = Dependovirus adeno-associated virus
DAAGKYT

SEQ ID NO: 2631 - protein, 10 aa, unidentified
  source 1..10: note = Dependovirus adeno-associated virus
EIKSGSVDGS

SEQ ID NO: 2632 - protein, 10 aa, unidentified
  source 1..10: note = Dependovirus adeno-associated virus
VFTLPQYGYA

SEQ ID NO: 2633 - protein, 9 aa, unidentified
  source 1..9: note = Dependovirus adeno-associated virus
STNNTGGVQ

SEQ ID NO: 2634 - skipped

SEQ ID NO: 2635 - protein, 7 aa, unidentified
  source 1..7: note = Dependovirus adeno-associated virus
SGVNRAS

SEQ ID NO: 2636 - protein, 7 aa, unidentified
  source 1..7: note = Dependovirus adeno-associated virus
LQGSNTY

SEQ ID NO: 2637 - protein, 8 aa, unidentified
  source 1..8: note = Dependovirus adeno-associated virus
ANPGTTAT

SEQ ID NO: 2638 - protein, 10 aa, unidentified
  source 1..10: note = Dependovirus adeno-associated virus
TTAPATGTYN

SEQ ID NO: 2639 - skipped

SEQ ID NO: 2640 - protein, 7 aa, unidentified
  source 1..7: note = Dependovirus adeno-associated virus
DSTGEYR

SEQ ID NO: 2641 - protein, 7 aa, unidentified
  source 1..7: note = Dependovirus adeno-associated virus
SASTGAS

SEQ ID NO: 2642 - protein, 10 aa, unidentified
  source 1..10: note = Dependovirus adeno-associated virus
VFMIPQYGYL

SEQ ID NO: 2643 - protein, 9 aa, unidentified
  source 1..9: note = Dependovirus adeno-associated virus
NQSGSAQNK

SEQ ID NO: 2644 - skipped

SEQ ID NO: 2645 - protein, 8 aa, unidentified
  source 1..8: note = Dependovirus adeno-associated virus
KTDNNNSN

SEQ ID NO: 2646 - protein, 7 aa, unidentified
  source 1..7: note = Dependovirus adeno-associated virus
KDDKDKF

SEQ ID NO: 2647 - protein, 6 aa, unidentified
  source 1..6: note = Dependovirus adeno-associated virus
SAGASN

SEQ ID NO: 2648 - protein, 10 aa, unidentified
  source 1..10: note = Dependovirus adeno-associated virus
STDPATGDVH

SEQ ID NO: 2649 - skipped

SEQ ID NO: 2650 - protein, 7 aa, unidentified
  source 1..7: note = Dependovirus adeno-associated virus
DNNGLYT

SEQ ID NO: 2651 - protein, 7 aa, unidentified
  source 1..7: note = Dependovirus adeno-associated virus
SETAGST

SEQ ID NO: 2652 - protein, 10 aa, unidentified
  source 1..10: note = Dependovirus adeno-associated virus
VFMIPQYGYL

SEQ ID NO: 2653 - protein, 9 aa, unidentified
  source 1..9: note = Dependovirus adeno-associated virus
NPGGTAGNR

SEQ ID NO: 2654 - skipped

SEQ ID NO: 2655 - protein, 8 aa, unidentified
  source 1..8: note = Dependovirus adeno-associated virus
LDQNNNSN

SEQ ID NO: 2656 - protein, 7 aa, unidentified
  source 1..7: note = Dependovirus adeno-associated virus
KDDEDRF

SEQ ID NO: 2657 - protein, 6 aa, unidentified
  source 1..6: note = Dependovirus adeno-associated virus
GATNKT

SEQ ID NO: 2658 - protein, 10 aa, unidentified
  source 1..10: note = Dependovirus adeno-associated virus
NTAAQTQVVN

SEQ ID NO: 2659 - skipped

SEQ ID NO: 2660 - protein, 7 aa, unidentified
  source 1..7: note = Dependovirus adeno-associated virus
DSQGVYS

SEQ ID NO: 2661 - protein, 8 aa, unidentified
  source 1..8: note = Dependovirus adeno-associated virus
NGTSGGAT

SEQ ID NO: 2662 - protein, 10 aa, unidentified
  source 1..10: note = Dependovirus adeno-associated virus
VFMIPQYGYL

SEQ ID NO: 2663 - protein, 9 aa, unidentified
  source 1..9: note = Dependovirus adeno-associated virus
TTGGTANTQ

SEQ ID NO: 2664 - skipped

SEQ ID NO: 2665 - protein, 8 aa, unidentified
  source 1..8: note = Dependovirus adeno-associated virus
TGQNNNSN

SEQ ID NO: 2666 - protein, 7 aa, unidentified
  source 1..7: note = Dependovirus adeno-associated virus
KDDEERF

SEQ ID NO: 2667 - protein, 6 aa, unidentified
  source 1..6: note = Dependovirus adeno-associated virus
NAARDN

SEQ ID NO: 2668 - protein, 11 aa, unidentified
  source 1..11: note = Dependovirus adeno-associated virus
NTAPQIGTVNS

SEQ ID NO: 2669 - skipped

SEQ ID NO: 2670 - protein, 7 aa, unidentified
  source 1..7: note = Dependovirus adeno-associated virus
NTEGVYS

SEQ ID NO: 2671 - protein, 8 aa, unidentified
  source 1..8: note = Dependovirus adeno-associated virus
NSTSGGSS

SEQ ID NO: 2672 - protein, 10 aa, unidentified
  source 1..10: note = Dependovirus adeno-associated virus
VFMIPQYGYL

SEQ ID NO: 2673 - protein, 9 aa, unidentified
  source 1..9: note = Dependovirus adeno-associated virus
INGSGQNQQ

SEQ ID NO: 2674 - skipped

SEQ ID NO: 2675 - protein, 8 aa, unidentified
  source 1..8: note = Dependovirus adeno-associated virus
VTQNNNSE

SEQ ID NO: 2676 - protein, 7 aa, unidentified
  source 1..7: note = Dependovirus adeno-associated virus
KEGEDRF

SEQ ID NO: 2677 - protein, 6 aa, unidentified
  source 1..6: note = Dependovirus adeno-associated virus
GTGRDN

SEQ ID NO: 2678 - protein, 10 aa, unidentified
  source 1..10: note = Dependovirus adeno-associated virus
QAQAQTGWVQ

SEQ ID NO: 2679 - skipped

SEQ ID NO: 2680 - protein, 7 aa, unidentified
  source 1..7: note = Dependovirus adeno-associated virus
NTEGVYS

SEQ ID NO: 2681 - protein, 8 aa, unidentified
  source 1..8: note = Dependovirus adeno-associated virus
NGTSGGST

SEQ ID NO: 2682 - protein, 10 aa, unidentified
  source 1..10: note = Dependovirus adeno-associated virus
VFMVPQYGYL

SEQ ID NO: 2683 - protein, 9 aa, unidentified
  source 1..9: note = Dependovirus adeno-associated virus
QTTGTGGTQ

SEQ ID NO: 2684 - skipped

SEQ ID NO: 2685 - protein, 8 aa, unidentified
  source 1..8: note = Dependovirus adeno-associated virus
TNQNNNSN

SEQ ID NO: 2686 - protein, 7 aa, unidentified
  source 1..7: note = Dependovirus adeno-associated virus
KDDDDRF

SEQ ID NO: 2687 - protein, 6 aa, unidentified
  source 1..6: note = Dependovirus adeno-associated virus
GAGNDG

SEQ ID NO: 2688 - protein, 10 aa, unidentified
  source 1..10: note = Dependovirus adeno-associated virus
NTQAQTGLVH

SEQ ID NO: 2689 - skipped

SEQ ID NO: 2690 - protein, 7 aa, unidentified
  source 1..7: note = Dependovirus adeno-associated virus
NTEGVYS

SEQ ID NO: 2691 - protein, 8 aa, unidentified
  source 1..8: note = Dependovirus adeno-associated virus
NGTSGGST

SEQ ID NO: 2692 - protein, 10 aa, unidentified
  source 1..10: note = Dependovirus adeno-associated virus
VFMIPQYGYL

SEQ ID NO: 2693 - protein, 9 aa, unidentified
  source 1..9: note = Dependovirus adeno-associated virus
STGGTAGTQ

SEQ ID NO: 2694 - skipped

SEQ ID NO: 2695 - protein, 8 aa, unidentified
  source 1..8: note = Dependovirus adeno-associated virus
LSQNNNSN

SEQ ID NO: 2696 - protein, 7 aa, unidentified
  source 1..7: note = Dependovirus adeno-associated virus
KDDEERF

SEQ ID NO: 2697 - protein, 6 aa, unidentified
  source 1..6: note = Dependovirus adeno-associated virus
GAGKDN

SEQ ID NO: 2698 - protein, 10 aa, unidentified
  source 1..10: note = Dependovirus adeno-associated virus
NAAPIVGAVN

SEQ ID NO: 2699 - skipped

SEQ ID NO: 2700 - protein, 7 aa, unidentified
  source 1..7: note = Dependovirus adeno-associated virus
NTDGTYS

SEQ ID NO: 2701 - protein, 8 aa, unidentified
  source 1..8: note = Dependovirus adeno-associated virus
NGTSGGST

SEQ ID NO: 2702 - protein, 10 aa, unidentified
  source 1..10: note = Dependovirus adeno-associated virus
VFMIPQYGYL

SEQ ID NO: 2703 - protein, 9 aa, unidentified
  source 1..9: note = Dependovirus adeno-associated virus
STGGTQGTQ

SEQ ID NO: 2704 - skipped

SEQ ID NO: 2705 - protein, 8 aa, unidentified
  source 1..8: note = Dependovirus adeno-associated virus
LSQNNNSN

SEQ ID NO: 2706 - protein, 7 aa, unidentified
  source 1..7: note = Dependovirus adeno-associated virus
KDDEERF

SEQ ID NO: 2707 - protein, 6 aa, unidentified
  source 1..6: note = Dependovirus adeno-associated virus
GAGRDN

SEQ ID NO: 2708 - protein, 10 aa, unidentified
  source 1..10: note = Dependovirus adeno-associated virus
NTGPIVGNVN

SEQ ID NO: 2709 - skipped

SEQ ID NO: 2710 - protein, 7 aa, unidentified
  source 1..7: note = Dependovirus adeno-associated virus
NTEGTYS

SEQ ID NO: 2711 - protein, 8 aa, unidentified
  source 1..8: note = Dependovirus adeno-associated virus
RLGTTSSS

SEQ ID NO: 2712 - protein, 10 aa, unidentified
  source 1..10: note = Dependovirus adeno-associated virus
VFMVPQYGYC

SEQ ID NO: 2713 - protein, 9 aa, unidentified
  source 1..9: note = Dependovirus adeno-associated virus
GETLNQGNA

SEQ ID NO: 2714 - skipped

SEQ ID NO: 2715 - protein, 12 aa, unidentified
  source 1..12: note = Dependovirus adeno-associated virus
ASQNYKIPASGG

SEQ ID NO: 2716 - protein, 7 aa, unidentified
  source 1..7: note = Dependovirus adeno-associated virus
GPSDGDF

SEQ ID NO: 2717 - protein, 6 aa, unidentified
  source 1..6: note = Dependovirus adeno-associated virus
VTGNTT

SEQ ID NO: 2718 - protein, 10 aa, unidentified
  source 1..10: note = Dependovirus adeno-associated virus
TTAPITGNVT

SEQ ID NO: 2719 - skipped

SEQ ID NO: 2720 - protein, 7 aa, unidentified
  source 1..7: note = Dependovirus adeno-associated virus
DTTGKYT

SEQ ID NO: 2721 - protein, 8 aa, unidentified
  source 1..8: note = Dependovirus adeno-associated virus
RIGTTANS

SEQ ID NO: 2722 - protein, 10 aa, unidentified
  source 1..10: note = Dependovirus adeno-associated virus
VFMVPQYGYC

SEQ ID NO: 2723 - protein, 9 aa, unidentified
  source 1..9: note = Dependovirus adeno-associated virus
GNSLNQGTA

SEQ ID NO: 2724 - skipped

SEQ ID NO: 2725 - protein, 12 aa, unidentified
  source 1..12: note = Dependovirus adeno-associated virus
ANQNYKIPASGG

SEQ ID NO: 2726 - protein, 7 aa, unidentified
  source 1..7: note = Dependovirus adeno-associated virus
GAGDSDF

SEQ ID NO: 2727 - protein, 6 aa, unidentified
  source 1..6: note = Dependovirus adeno-associated virus
PSGNTT

SEQ ID NO: 2728 - protein, 10 aa, unidentified
  source 1..10: note = Dependovirus adeno-associated virus
TTAPHIANLD

SEQ ID NO: 2729 - skipped

SEQ ID NO: 2730 - protein, 7 aa, unidentified
  source 1..7: note = Dependovirus adeno-associated virus
DNAGNYH

SEQ ID NO: 2731 - protein, 8 aa, unidentified
  source 1..8: note = Dependovirus adeno-associated virus
RLGTTSNS

SEQ ID NO: 2732 - protein, 10 aa, unidentified
  source 1..10: note = Dependovirus adeno-associated virus
VFMVPQYGYC

SEQ ID NO: 2733 - protein, 9 aa, unidentified
  source 1..9: note = Dependovirus adeno-associated virus
GETLNQGNA

SEQ ID NO: 2734 - skipped

SEQ ID NO: 2735 - protein, 12 aa, unidentified
  source 1..12: note = Dependovirus adeno-associated virus
ASQNYKIPASGG

SEQ ID NO: 2736 - protein, 7 aa, unidentified
  source 1..7: note = Dependovirus adeno-associated virus
GPSDGDF

SEQ ID NO: 2737 - protein, 6 aa, unidentified
  source 1..6: note = Dependovirus adeno-associated virus
VTGNTT

SEQ ID NO: 2738 - protein, 10 aa, unidentified
  source 1..10: note = Dependovirus adeno-associated virus
TTAPITGNVT

SEQ ID NO: 2739 - skipped

SEQ ID NO: 2740 - protein, 7 aa, unidentified
  source 1..7: note = Dependovirus adeno-associated virus
DTTGKYT

SEQ ID NO: 2741 - protein, 8 aa, unidentified
  source 1..8: note = Dependovirus adeno-associated virus
RLGSSNAS

SEQ ID NO: 2742 - protein, 10 aa, unidentified
  source 1..10: note = Dependovirus adeno-associated virus
VFMVPQYGYC

SEQ ID NO: 2743 - protein, 9 aa, unidentified
  source 1..9: note = Dependovirus adeno-associated virus
GGTLNQGNS

SEQ ID NO: 2744 - skipped

SEQ ID NO: 2745 - protein, 12 aa, unidentified
  source 1..12: note = Dependovirus adeno-associated virus
ASQNYKIPQGRN

SEQ ID NO: 2746 - protein, 7 aa, unidentified
  source 1..7: note = Dependovirus adeno-associated virus
ANDATDF

SEQ ID NO: 2747 - protein, 6 aa, unidentified
  source 1..6: note = Dependovirus adeno-associated virus
ITGNTT

SEQ ID NO: 2748 - protein, 10 aa, unidentified
  source 1..10: note = Dependovirus adeno-associated virus
TTVPTVDDVD

SEQ ID NO: 2749 - skipped

SEQ ID NO: 2750 - protein, 7 aa, unidentified
  source 1..7: note = Dependovirus adeno-associated virus
DNAGAYK

SEQ ID NO: 2751 - protein, 8 aa, unidentified
  source 1..8: note = Dependovirus adeno-associated virus
RIQGPSGG

SEQ ID NO: 2752 - protein, 10 aa, unidentified
  source 1..10: note = Dependovirus adeno-associated virus
IYTIPQYGYC

SEQ ID NO: 2753 - protein, 9 aa, unidentified
  source 1..9: note = Dependovirus adeno-associated virus
VSQAGSSGR

SEQ ID NO: 2754 - skipped

SEQ ID NO: 2755 - protein, 12 aa, unidentified
  source 1..12: note = Dependovirus adeno-associated virus
ASNITKNNVFSV

SEQ ID NO: 2756 - protein, 7 aa, unidentified
  source 1..7: note = Dependovirus adeno-associated virus
FSGEPDR

SEQ ID NO: 2757 - protein, 8 aa, unidentified
  source 1..8: note = Dependovirus adeno-associated virus
VYDQTTAT

SEQ ID NO: 2758 - protein, 10 aa, unidentified
  source 1..10: note = Dependovirus adeno-associated virus
VTPGTRAAVN

SEQ ID NO: 2759 - skipped

SEQ ID NO: 2760 - protein, 7 aa, unidentified
  source 1..7: note = Dependovirus adeno-associated virus
SDTGSYS

(19) United States
(12) Patent Application Publication
(10) Pub. No.: US 2024/0067986 A1
(43) Pub. Date: Feb. 29, 2024

(54) RECOMBINANT ADENO-ASSOCIATED VIRUS VECTORS

(71) Applicant: Ginkgo Bioworks, Inc., Boston, MA (US)

(72) Inventors: Daniel McCoy, Durham, NC (US); Garrett E. Berry, Durham, NC (US)

(21) Appl. No.: 18/372,334

(22) Filed: Sep. 25, 2023

Related U.S. Application Data
(63) Continuation of application No. 17/479,577, filed on Sep. 20, 2021.
Application No. 17/479,577 is a continuation of application No. PCT/US2020/023877, filed on Mar. 20, 2020.
(60) Provisional application No. 62/821,710, filed on Mar. 21, 2019.

(51) Int. Cl. C12N 15/86; C07K 14/005

(52) U.S. Cl. CPC C12N 15/86; C07K 14/005; A61K 48/00

(57) ABSTRACT

AAV capsid proteins comprising a modification in the amino acid sequence and virus vectors comprising the modified AAV capsid protein are described. Also described are methods of administering the virus vectors and virus capsids to a cell or to a subject in vivo.

### CROSS-REFERENCE TO RELATED APPLICATION

This application claims the benefit of U.S. Provisional Application No. 62/821,710, filed Mar. 21, 2019, which is incorporated herein by reference in its entirety.

### DESCRIPTION OF THE TEXT FILE SUBMITTED ELECTRONICALLY

The contents of the text file submitted electronically herewith are incorporated by reference in their entirety: a computer readable format copy of the Sequence Listing (filename: STRD_013_01WO_SeqList_ST25.txt, date recorded Mar. 20, 2020, file size ˜350 kilobytes).

### TECHNICAL FIELD

This application relates to adeno-associated virus (AAV) vectors comprising recombinant capsid proteins. In some embodiments, the recombinant AAV vectors evade neutralizing antibodies without decreased transduction efficiency.

### BACKGROUND

Host-derived pre-existing antibodies generated upon natural encounter of AAV or recombinant AAV vectors prevent first time as well as repeat administration of AAV vectors as vaccines and/or for gene therapy. Serological studies reveal a high prevalence of antibodies in the human population worldwide with about 67% of people having antibodies against AAV1, 72% against AAV2, and about 40% against AAV5 through AAV9.

In gene therapy, certain clinical scenarios involving gene silencing or tissue degeneration may require multiple AAV vector administrations to sustain long term expression of the transgene. Accordingly, there is a need in the art for recombinant AAV vectors which evade antibody recognition. Such vectors will help a) expand the eligible cohort of subjects suitable for AAV-based gene therapy and b) allow multiple, repeat administrations of AAV-based gene therapy vectors.

### BRIEF SUMMARY

Provided herein are recombinant AAV vectors which evade antibody recognition and/or selectively target tissues of the CNS.

In some embodiments, an adeno-associated virus (AAV) vector comprises (i) a recombinant capsid protein and (ii) a cargo nucleic acid encapsidated by the capsid protein, wherein the capsid protein comprises a peptide having the sequence of any one of SEQ ID NO: 12-20.

In some embodiments, an adeno-associated virus (AAV) vector comprises (i) a mutant AAV9 capsid protein and (ii) a cargo nucleic acid encapsidated by the capsid protein, wherein the capsid protein comprises a peptide having the sequence X¹-X²-X³-X⁴-X⁵-X⁶-X⁷-X⁸ (SEQ ID NO: 158) at amino acids 451-458 of the native AAV9 capsid protein sequence, wherein the peptide does not occur in the native AAV9 capsid protein sequence.

In some embodiments, an adeno-associated virus (AAV) vector comprises (i) a mutant AAV9 capsid protein and (ii) a cargo nucleic acid encapsidated by the capsid protein, wherein the capsid protein comprises a peptide having the sequence X¹-X²-X³-X⁴-X⁵-X⁶-X⁷-X⁸ (SEQ ID NO: 158) at amino acids 587-594 of the native AAV9 capsid protein sequence, wherein the peptide does not occur in the native AAV9 capsid protein sequence.

In some embodiments, the an adeno-associated virus (AAV) vector comprises (i) a recombinant capsid protein and (ii) a cargo nucleic acid encapsidated by the capsid protein, wherein the capsid protein comprises an amino acid sequence that is at least 95%, at least 96%, at least 97%, at least 98%, or at least 99% identical to any one of SEQ ID NO: 165-187.

Also provided herein are nucleic acid sequences encoding a recombinant capsid protein, and expression vectors comprising the same.

Also provided are cells comprising a nucleic acid, an expression vector, an AAV vector, or an AAV capsid described herein.

Also provided are pharmaceutical compositions comprising a nucleic acid, an expression vector, an AAV vector, an AAV capsid, or a cell described herein.

Also provided are methods for treating a subject in need thereof comprising administering to the subject a therapeutically effective amount of an AAV vector described herein.

Also provided are in vitro method of introducing a nucleic acid molecule into a cell, the methods comprising contacting the cell with an AAV vector described herein.

Also provided is an AAV vector described herein for use as a medicament.

Also provided is an AAV vector described herein for use in a method of treatment of a subject in need thereof.

Also provided is an AAV vector described herein for use in a method of treating or preventing a disease or disorder of the CNS in a subject in need thereof.

These and other embodiments are described in more detail below.

### BRIEF DESCRIPTION OF THE DRAWINGS

FIG. 1A-1C. Bubble plots showing analysis of library diversity, directed evolution and enrichment of novel antigenic footprints. Parental (FIG. 1A) and evolved libraries from a first round (FIG. 1B) and a second round (FIG. 1C) of evolution were subjected to high-throughput sequencing using the Illumina MiSeq platform. Following analysis with a custom Perl script, enriched amino acid sequences were plotted. Each bubble represents a distinct capsid amino acid sequence with the radius of the bubble proportional to the number of reads for that variant in the respective library. The y-axis represents the percentage of total reads from the sequencing run. Data are spread along the x-axis for ease of visualization. The percent reduction in unique clones (96.5%) directly demonstrates that numerous “un-fit” sequences were removed after a first and second round of evolution. Dominant isolates were selected for further analysis.

FIG. 2. Volumetric yield of AAV vectors comprising AAV capsid variants STRD.101 and STRD.102, as compared to wildtype AAV9. Bars represent mean+/−95% confidence interval.

FIG. 3. Infectivity values of AAV-STRD.101 and wildtype AAV9 determined using a standard TCID50 assay. Data are graphed as the natural log of the number of particles required to generate an infectious unit (P:I Ratio). Error bars represent standard deviation.

FIG. 4A-4D. Transduction of U87 cells (FIG. 4A), N2A cells (FIG. 4B), Sy5Y cells (FIG. 4C), and U2OS cells (FIG. 4D) by recombinant AAV vectors comprising the STRD.101 capsid and packaging a luciferase transgene, as compared to wildtype AAV9 vectors similarly packaging a luciferase sequence. Error bars represent standard error.

FIG. 5. Representative fluorescent microscopy images showing tdTomato expression in coronal vibratome sections 24 hours post-fixation with 4% PFA. Each section is 25 μm thick. Top panel shows images obtained using a 4× objective lens with native tdTomato fluorescence. The bottom panel shows images obtained using a 10× objective lens with native tdTomato fluorescence.

FIG. 6. Representative immunohistochemistry images showing tdTomato expression in coronal vibratome sections 24 hours post-fixation with 4% PFA. Each section is 25 μm thick.

FIG. 7. Representative fluorescent microscopy images showing TdTomato expression in vibratrome liver sections 24 hours post-fixation with 4% PFA. Each section is 25 μm in thick. Panels show native tdTomato fluorescence with DAPI counterstain.

FIG. 8. Representative fluorescent microscopy images showing TdTomato expression in vibratrome heart sections 24 hours post-fixation with 4% PFA. Each section is 50 μm in thick. Panels show native tdTomato fluorescence with DAPI counterstain.

FIG. 9. Biodistribution of recombinant AAVs in non-human primates. Horizontal line shows limit of detection.

### DETAILED DESCRIPTION

Unless otherwise defined, all technical and scientific terms used herein have the same meaning as commonly understood by one of ordinary skill in the art to which this disclosure belongs. The terminology used in the detailed description herein is for the purpose of describing particular embodiments only and is not intended to be limiting.

All publications, patent applications, patents, GenBank or other accession numbers and other references mentioned herein are incorporated by reference in their entirety for all purposes.

The designation of amino acid positions in the AAV capsid proteins in the disclosure and the appended claims is with respect to VP1 capsid subunit numbering. It will be understood by those skilled in the art that the modifications described herein if inserted into the AAV cap gene may result in modifications in the VP1, VP2 and/or VP3 capsid subunits. Alternatively, the capsid subunits can be expressed independently to achieve modification in only one or two of the capsid subunits (VP1, VP2, VP3, VP1+VP2, VP1+VP3, or VP2+VP3).

### Definitions

The following terms are used in the description herein and the appended claims.

The singular forms “a,” “an” and “the” are intended to include the plural forms as well, unless the context clearly indicates otherwise.

Furthermore, the term “about” as used herein when referring to a measurable value such as an amount of the length of a polynucleotide or polypeptide sequence, dose, time, temperature, and the like, is meant to encompass variations of ±20%, 10%, 5%, 1%, +0.5%, or even ±0.1% of the specified amount.

Also as used herein, “and/or” refers to and encompasses any and all possible combinations of one or more of the associated listed items, as well as the lack of combinations when interpreted in the alternative (“or”).

Unless the context indicates otherwise, it is specifically intended that the various features described herein can be used in any combination. Moreover, in some embodiments, any feature or combination of features set forth herein can be excluded or omitted. To illustrate further, if, for example, the specification indicates that a particular amino acid can be selected from A, G, I, L and/or V, this language also indicates that the amino acid can be selected from any subset of these amino acid(s) for example A, G, I or L; A, G, I or V; A or G; only L; etc., as if each such subcombination is expressly set forth herein. Moreover, such language also indicates that one or more of the specified amino acids can be disclaimed. For example, in some embodiments the amino acid is not A, G or I; is not A; is not G or V; etc., as if each such possible disclaimer is expressly set forth herein.

As used herein, the terms “reduce,” “reduces,” “reduction” and similar terms mean a decrease of at least about 10%, about 15%, about 20%, about 25%, about 35%, about 50%, about 75%, about 80%, about 85%, about 90%, about 95%, about 97% or more.

As used herein, the terms “increase,” “improve,” “enhance,” “enhances,” “enhancement” and similar terms indicate an increase of at least about 10%, about 15%, about 20%, about 25%, about 50%, about 75%, about 100%, about 150%, about 200%, about 300%, about 400%, about 500% or more.

The term “parvovirus” as used herein encompasses the family Parvoviridae, including autonomously replicating parvoviruses and dependoviruses. The autonomous parvoviruses include members of the genera Protoparvovirus, Erythroparvovirus, Bocaparvovirus, and Densovirus subfamily. Exemplary autonomous parvoviruses include, but are not limited to, minute virus of mouse, bovine parvovirus, canine parvovirus, chicken parvovirus, feline panleukopenia virus, feline parvovirus, goose parvovirus, H1 parvovirus, muscovy duck parvovirus, B19 virus, and any other autonomous parvovirus now known or later discovered. Other autonomous parvoviruses are known to those skilled in the art. See, e.g., BERNARD N. FIELDS et al, VIROLOGY, volume 2, chapter 69 (4th ed., Lippincott-Raven Publishers; Cotmore et al. Archives of Virology DOI 10.1007/s00705-013-1914-1).

As used herein, the term “adeno-associated virus” (AAV), includes but is not limited to, AAV type 1, AAV type 2, AAV type 3 (including types 3A and 3B), AAV type 4, AAV type 5, AAV type 6, AAV type 7, AAV type 8, AAV type 9, AAV type 10, AAV type 11, AAV type 12, AAV type 13, AAV type rh32.33, AAV type rh8, AAV type rh10, AAV type rh74, AAV type hu.68, avian AAV, bovine AAV, canine AAV, equine AAV, ovine AAV, snake AAV, bearded dragon AAV, AAV2i8, AAV2g9, AAV-LK03, AAV7m8, AAV Anc80, AAV PHP.B, and any other AAV now known or later discovered. See, e.g., BERNARD N. FIELDS et al., VIROLOGY, volume 2, chapter 69 (4th ed., Lippincott-Raven Publishers). A number of AAV serotypes and clades have been identified (see, e.g., Gao et al, (2004) J. Virology 78:6381-6388; Moris et al, (2004) Virology 33-:375-383; and Table 2). Exemplary AAV capsid sequences for AAV1-9, AAVrh.10 and AAV11 are provided in SEQ ID NO: 1-11.

As used herein, the term “chimeric AAV” refers to an AAV comprising a capsid protein with regions, domains, individual amino acids that are derived from two or more different serotypes of AAV. In some embodiments, a chimeric AAV comprises a capsid protein comprised of a first region that is derived from a first AAV serotype and a second region that is derived from a second AAV serotype. In some embodiments, a chimeric AAV comprises a capsid protein comprised of a first region that is derived from a first AAV serotype, a second region that is derived from a second AAV serotype, and a third region that is derived from a third AAV serotype. In some embodiments, the chimeric AAV may comprise regions, domains, individual amino acids derived from AAV1, AAV2, AAV3, AAV4, AAV5, AAV6, AAV7, AAV8, AAV9, AAV10, AAV11, and/or AAV12. For example, the chimeric AAV may include regions, domains, and/or individual amino acids from a first and a second AAV serotype as shown below (Table 1), wherein AAVX+Y indicates a chimeric AAV including sequences derived from AAVX and AAVY:

TABLE 1
Chimeric AAVs

| | | Second AAV Serotype | | | | | | |
|---|---|---|---|---|---|---|---|---|
| | | AAV1 | AAV2 | AAV3 | AAV4 | AAV5 | AAV6 | AAV7 |
| First | AAV1 | x | AAV1 + 2 | AAV1 + 3 | AAV1 + 4 | AAV1 + 5 | AAV1 + 6 | AAV1 + 7 |
| AAV | AAV2 | AAV2 + 1 | x | AAV2 + 3 | AAV2 + 4 | AAV2 + 5 | AAV2 + 6 | AAV2 + 7 |
| Sertoype | AAV3 | AAV3 + 1 | AAV3 + 2 | x | AAV3 + 4 | AAV3 + 5 | AAV3 + 6 | AAV3 + 7 |
| | AAV4 | AAV4 + 1 | AAV4 + 2 | AAV4 + 3 | x | AAV4 + 5 | AAV4 + 6 | AAV4 + 7 |
| | AAV5 | AAV5 + 1 | AAV5 + 2 | AAV5 + 3 | AAV5 + 4 | x | AAV5 + 6 | AAV5 + 7 |
| | AAV6 | AAV6 + 1 | AAV6 + 2 | IAAV6 + 3 | AAV6 + 4 | AAV6 + 5 | x | AAV6 + 7 |
| | AAV7 | AAV7 + 1 | AAV7 + 2 | AAV7 + 3 | AAV7 + 4 | AAV7 + 5 | AAV7 + 6 | x |
| | AAV8 | AAV8 + 1 | AAV8 + 2 | AAV8 + 3 | AAV8 + 4 | AAV8 + 5 | AAV8 + 6 | AAV8 + 7 |
| | AAV9 | AAV9 + 1 | AAV9 + 2 | AAV9 + 3 | AAV9 + 4 | AAV9 + 5 | AAV9 + 6 | AAV9 + 7 |
| | AAV10 | AAV10 + 1 | AAV10 + 2 | AAV10 + 3 | AAV10 + 4 | AAV10 + 5 | AAV10 + 6 | AAV10 + 7 |
| | AAV11 | AAV11 + 1 | AAV11 + 2 | AAV11 + 3 | AAV11 + 4 | AAV11 + 5 | AAV11 + 6 | AAV11 + 7 |
| | AAV12 | AAV12 + 1 | AAV12 + 2 | AAV12 + 3 | AAV12 + 4 | AAV12 + 5 | AAV12 + 6 | AAV12 + 7 |

| | | Second AAV Serotype | | | | |
|---|---|---|---|---|---|---|
| | | AAV8 | AAV9 | AAV10 | AAV11 | AAV12 |
| First | AAV1 | AAV1 + 8 | AAV1 + 9 | AAV1 + 10 | AAV1 + 11 | AAV1 + 12 |
| AAV | AAV2 | AAV2 + 8 | AAV2 + 9 | AAV2 + 10 | AAV2 + 11 | AAV2 + 12 |
| Sertoype | AAV3 | AAV3 + 8 | AAV3 + 9 | AAV3 + 10 | AAV3 + 11 | AAV3 + 12 |
| | AAV4 | AAV4 + 8 | AAV4 + 9 | AAV4 + 10 | AAV4 + 11 | AAV4 + 12 |
| | AAV5 | AAV5 + 8 | AAV5 + 9 | AAV5 + 10 | AAV5 + 11 | AAV5 + 12 |
| | AAV6 | AAV6 + 8 | AAV6 + 9 | AAV6 + 10 | AAV6 + 11 | AAV6 + 12 |
| | AAV7 | AAV7 + 8 | AAV7 + 9 | AAV7 + 10 | AAV7 + 11 | AAV7 + 12 |
| | AAV8 | x | AAV8 + 9 | AAV8 + 10 | AAV8 + 11 | AAV8 + 12 |
| | AAV9 | AAV9 + 8 | x | AAV9 + 10 | AAV9 + 11 | AAV9 + 12 |
| | AAV10 | AAV10 + 8 | AAV10 + 9 | x | AAV10 + 11 | AAV10 + 12 |
| | AAV11 | AAV11 + 8 | AAV11 + 9 | AAV11 + 10 | x | AAV11 + 12 |
| | AAV12 | AAV12 + 8 | AAV12 + 9 | AAV12 + 10 | AAV12 + 11 | x |

By including individual amino acids or regions from multiple AAV serotypes in one capsid protein, capsid proteins that have multiple desired properties that are separately derived from the multiple AAV serotypes may be obtained.

The genomic sequences of various serotypes of AAV and the autonomous parvoviruses, as well as the sequences of the native terminal repeats (TRs), Rep proteins, and capsid subunits are known in the art. Such sequences may be found in the literature or in public databases such as GenBank. See, e.g., GenBank Accession Numbers NC_002077, NC_001401, NC_001729, NC_001863, NC_001829, NC_001862, NC_000883, NC_001701, NC_001510, NC_006152, NC_006261, AF063497, U89790, AF043303, AF028705, AF028704, J02275, J01901, J02275, X01457, AF288061, AH009962, AY028226, AY028223, NC_001358, NC_001540, AF513851, AF513852, AY530579; the disclosures of which are incorporated by reference herein for teaching parvovirus and AAV nucleic acid and amino acid sequences. See also, e.g., Srivistava et al., (1983) J. Virology 45:555; Chiorini et al, (1998) J Virology 71:6823; Chiorini et al., (1999) J. Virology 73: 1309; Bantel-Schaal et al., (1999) J Virology 73:939; Xiao et al, (1999) J Virology 73:3994; Muramatsu et al., (1996) Virology 221:208; Shade et al, (1986) J. Virol. 58:921; Gao et al, (2002) Proc. Nat. Acad. Sci. USA 99:11854; Moris et al, (2004) Virology 33:375-383; international patent publications WO 00/28061, WO 99/61601, WO 98/11244; and U.S. Pat. No. 6,156,303; the disclosures of which are incorporated by reference herein for teaching parvovirus and AAV nucleic acid and amino acid sequences. See also Table 2. The capsid structures of autonomous parvoviruses and AAV are described in more detail in BERNARD N. FIELDS et al., VIROLOGY, volume 2, chapters 69 & 70 (4th ed., Lippincott-Raven Publishers). See also, description of the crystal structure of AAV2 (Xie et al., (2002) Proc. Nat. Acad. Sci. 99: 10405-10), AAV9 (DiMattia et al., (2012) J. Virol. 86:6947-6958), AAV8 (Nam et al, (2007) J. Virol. 81: 12260-12271), AAV6 (Ng et al., (2010) J. Virol. 84:12945-12957), AAV5 (Govindasamy et al. (2013) J. Virol. 87, 11187-11199), AAV4 (Govindasamy et al. (2006) J. Virol. 80:11556-11570), AAV3B (Lerch et al., (2010) Virology 403:26-36), BPV (Kailasan et al., (2015) J. Virol. 89:2603-2614) and CPV (Xie et al, (1996) J. Mol. Biol. 6:497-520 and Tsao et al, (1991) Science 251:1456-64).

TABLE 2
AAV Serotypes and Clades

| | GenBank |
|---|---|
| | Accession |
| | Number |
| Complete | |
| Genomes | |
| Adeno-associated | NC_002077, |
| virus 1 | AF063497 |
| Adeno-associated | NC_001401 |
| virus 2 | |
| Adeno-associated | NC_001729 |
| virus 3 | |
| Adeno-associated | NC_001863 |
| virus 3B | |
| Adeno-associated | NC_001829 |
| virus 4 | |
| Adeno-associated | Y18065, |
| virus 5 | AF085716 |
| Adeno-associated | NC_001862 |
| virus 6 | |
| Avian AAV ATCC | AY186198, |
| VR-865 | AY629583, |
| | NC_004828 |
| Avian AAV strain | NC_006263, |
| DA-1 | AY629583 |
| Bovine AAV | NC_005889, |
| | AY388617, |
| | AAR26465 |
| AAV11 | AAT46339, |
| | AY631966 |
| AAV12 | ABI16639, |
| | DQ813647 |
| Clade A | |
| AAV1 | NC_002077, |
| | AF063497 |
| AAV6 | NC_001862 |
| Hu.48 | AY530611 |
| Hu 43 | AY530606 |
| Hu 44 | AY530607 |
| Hu 46 | AY530609 |
| Clade B | |
| Hu. 19 | AY530584 |
| Hu. 20 | AY530586 |
| Hu 23 | AY530589 |
| Hu22 | AY530588 |
| Hu24 | AY530590 |
| Hu21 | AY530587 |
| Hu27 | AY530592 |
| Hu28 | AY530593 |
| Hu 29 | AY530594 |
| Hu63 | AY530624 |
| Hu64 | AY530625 |
| Hu13 | AY530578 |
| Hu56 | AY530618 |
| Hu57 | AY530619 |
| Hu49 | AY530612 |
| Hu58 | AY530620 |
| Hu34 | AY530598 |
| Hu35 | AY530599 |
| AAV2 | NC_001401 |
| Hu45 | AY530608 |
| Hu47 | AY530610 |
| Hu51 | AY530613 |
| Hu52 | AY530614 |
| Hu T41 | AY695378 |
| Hu S17 | AY695376 |
| Hu T88 | AY695375 |
| Hu T71 | AY695374 |
| Hu T70 | AY695373 |
| Hu T40 | AY695372 |
| Hu T32 | AY695371 |
| Hu T17 | AY695370 |
| Hu LG15 | AY695377 |
| Clade C | |
| Hu9 | AY530629 |
| Hu10 | AY530576 |
| Hu11 | AY530577 |
| Hu53 | AY530615 |
| Hu55 | AY530617 |
| Hu54 | AY530616 |
| Hu7 | AY530628 |
| Hu18 | AY530583 |
| Hu15 | AY530580 |
| Hu16 | AY530581 |
| Hu25 | AY530591 |
| Hu60 | AY530622 |
| Ch5 | AY243021 |
| Hu3 | AY530595 |
| Hu1 | AY530575 |
| Hu4 | AY530602 |
| Hu2 | AY530585 |
| Hu61 | AY530623 |
| Clade D | |
| Rh62 | AY530573 |
| Rh48 | AY530561 |
| Rh54 | AY530567 |
| Rh55 | AY530568 |
| Cy2 | AY243020 |
| AAV7 | AF513851 |
| Rh35 | AY243000 |
| Rh37 | AY242998 |
| Rh36 | AY242999 |
| Cy6 | AY243016 |
| Cy4 | AY243018 |
| Cy3 | AY243019 |
| Cy5 | AY243017 |
| Rh13 | AY243013 |
| Clade E | |
| Rh38 | AY530558 |
| Hu66 | AY530626 |
| Hu42 | AY530605 |
| Hu67 | AY530627 |
| Hu40 | AY530603 |
| Hu41 | AY530604 |
| Hu37 | AY530600 |
| Rh40 | AY530559 |
| Rh2 | AY243007 |
| Bb1 | AY243023 |
| Bb2 | AY243022 |
| Rh10 | AY243015 |
| Hu17 | AY530582 |
| Hu6 | AY530621 |
| Rh25 | AY530557 |
| Pi2 | AY530554 |
| Pi1 | AY530553 |
| Pi3 | AY530555 |
| Rh57 | AY530569 |
| Rh50 | AY530563 |
| Rh49 | AY530562 |
| Hu39 | AY530601 |
| Rh58 | AY530570 |
| Rh61 | AY530572 |
| Rh52 | AY530565 |
| Rh53 | AY530566 |
| Rh51 | AY530564 |
| Rh64 | AY530574 |
| Rh43 | AY530560 |
| AAV8 | AF513852 |
| Rh8 | AY242997 |
| Rh1 | AY530556 |
| Clade F | |
| Hu14 | AY530579 |
| (AAV9) | |
| Hu31 | AY530596 |
| Hu32 | AY530597 |
| HSC1 | MI332400.1 |
| HSC2 | MI332401.1 |
| HSC3 | MI332402.1 |
| HSC4 | MI332403.1 |
| HSC5 | MI332405.1 |
| HSC6 | MI332404.1 |
| HSC7 | MI332407.1 |
| HSC8 | MI332408.1 |
| HSC9 | MI332409.1 |
| HSC11 | MI332406.1 |
| HSC12 | MI332410.1 |
| HSC13 | MI332411.1 |
| HSC14 | MI332412.1 |
| HSC15 | MI332413.1 |
| HSC16 | MI332414.1 |
| HSC17 | MI332415.1 |
| Hu68 | |
| Clonal | |
| Isolate | |
| AAV5 | Y18065, |
| | AF085716 |
| AAV 3 | NC_001729 |
| AAV 3B | NC_001863 |
| AAV4 | NC_001829 |
| Rh34 | AY243001 |
| Rh33 | AY243002 |
| Rh32 | AY243003 |
| Others | |
| Rh74 | |
| Bearded | |
| Dragon | |
| AAV | |
| Snake | NC_006148.1 |
| AAV | |

The term “self-complimentary AAV” or “scAAV” refers to a recombinant AAV vector which forms a dimeric inverted repeat DNA molecule that spontaneously anneals, resulting in earlier and more robust transgene expression compared with conventional single-strand (ss) AAV genomes. See, e.g., McCarty, D. M., et al., Gene Therapy 8, 1248-1254 (2001). Unlike conventional ssAAV, scAAV can bypass second-strand synthesis, the rate-limiting step for gene expression. Moreover, double-stranded scAAV is less prone to DNA degradation after viral transduction, thereby increasing the number of copies of stable episomes. Notably, scAAV can typically only hold a genome that is about 2.4 kb, half the size of a conventional AAV vector. In some embodiments, the AAV vectors described herein are self-complementary AAVs.

As used herein, the term “peptide” refers to a short amino acid sequence. The term peptide may be used to refer to portion or region of an AAV capsid amino acid sequence. The peptide may be a peptide that naturally occurs in a native AAV capsid, or a peptide that does not naturally occur in a native AAV capsid. Naturally occurring AAV peptides in an AAV capsid may be substituted by non-naturally occurring peptides. For example, a non-naturally occurring peptide may be substituted into an AAV capsid to provide a modified capsid, such that the naturally-occurring peptide is replaced by the non-naturally occurring peptide.

The term “tropism” as used herein refers to preferential entry of the virus into certain cells or tissues, optionally followed by expression (e.g., transcription and, optionally, translation) of a sequence(s) carried by the viral genome in the cell, e.g., for a recombinant virus, expression of a transgene of interest.

As used here, “systemic tropism” and “systemic transduction” (and equivalent terms) indicate that the virus capsid or virus vector as described herein exhibits tropism for or transduces, respectively, tissues throughout the body (e.g., brain, lung, skeletal muscle, heart, liver, kidney and/or pancreas). In some embodiments, systemic transduction of muscle tissues (e.g., skeletal muscle, diaphragm and cardiac muscle) is achieved. In some embodiments, systemic transduction of skeletal muscle tissues is achieved. For example, in some embodiments, essentially all skeletal muscles throughout the body are transduced (although the efficiency of transduction may vary by muscle type). In some embodiments, systemic transduction of limb muscles, cardiac muscle and diaphragm muscle is achieved. Optionally, the virus capsid or virus vector is administered via a systemic route (e.g., systemic route such as intravenously, intra-articularly or intra-lymphatically).

Alternatively, in some embodiments, the capsid or virus vector is delivered locally (e.g., to the footpad, intramuscularly, intradermally, subcutaneously, topically). In some embodiments, the capsid or virus vector is delivered locally to a tissue of the central nervous system (CNS), such as the brain or the spinal cord. In some embodiments, the capsid or virus vector is administered by intrathecal, intracerebral or intracerebroventricular injection.

Unless indicated otherwise, “efficient transduction” or “efficient tropism,” or similar terms, can be determined by reference to a suitable control (e.g., at least about 50%, about 60%, about 70%, about 80%, about 85%, about 90%, about 95% or more of the transduction or tropism, respectively, of the control). In some embodiments, the virus vector (e.g., the AVV vector) efficiently transduces or has efficient tropism for skeletal muscle, cardiac muscle, diaphragm muscle, pancreas (including β-islet cells), spleen, the gastrointestinal tract (e.g., epithelium and/or smooth muscle), cells of the central nervous system, lung, joint cells, and/or kidney. Suitable controls will depend on a variety of factors including the desired tropism profile. For example, AAV8 and AAV9 are highly efficient in transducing skeletal muscle, cardiac muscle and diaphragm muscle, but have the disadvantage of also transducing liver with high efficiency. Thus, viral vectors can be identified that demonstrate the efficient transduction of skeletal, cardiac and/or diaphragm muscle of AAV8 or AAV9, but with a much lower transduction efficiency for liver. Further, because the tropism profile of interest may reflect tropism toward multiple target tissues, it will be appreciated that a suitable vector may represent some tradeoffs. To illustrate, a virus vector may be less efficient than AAV8 or AAV9 in transducing skeletal muscle, cardiac muscle and/or diaphragm muscle, but because of low level transduction of liver, may nonetheless be very desirable.

Similarly, it can be determined if a virus “does not efficiently transduce” or “does not have efficient tropism” for a target tissue, or similar terms, by reference to a suitable control. In some embodiments, the virus vector does not efficiently transduce (i.e., does not have efficient tropism) for liver, kidney, gonads and/or germ cells. In some embodiments, undesirable transduction of tissue(s) (e.g., liver) is about 20% or less, about 10% or less, about 5% or less, about 1% or less, about 0.1% or less of the level of transduction of the desired target tissue(s) (e.g., skeletal muscle, diaphragm muscle, cardiac muscle and/or cells of the central nervous system).

As used herein in connection with an AAV vector (or a capsid protein or peptide thereof), the terms “selectively binds,” “selective binding” and similar terms, refer to binding of the AAV vector (or a capsid protein or peptide thereof) to a target in a manner dependent upon the presence of a particular molecular structure. In some embodiments, selective binding refers to binding of the AAV predominantly to a specific target, without substantial or significant binding to other targets. In some embodiments, an AAV vector (or capsid protein or peptide thereof) specifically binds to a receptor in a cell or tissue of interest, but does not exhibit substantial or significant binding to other receptors.

A “polynucleotide” is a sequence of nucleotide bases, and may be RNA, DNA or DNA-RNA hybrid sequences (including both naturally occurring and non-naturally occurring nucleotide). In some embodiments, a polynucleotide is either a single or double stranded DNA sequence.

As used herein, an “isolated” polynucleotide (e.g., an “isolated DNA” or an “isolated RNA”) means a polynucleotide at least partially separated from at least some of the other components of the naturally occurring organism or virus, for example, the cell or viral structural components or other polypeptides or nucleic acids commonly found associated with the polynucleotide. In some embodiments an “isolated” nucleotide is enriched by at least about 10-fold, about 100-fold, about 1000-fold, about 10,000-fold or more as compared with the starting material.

Likewise, an “isolated” polypeptide means a polypeptide that is at least partially separated from at least some of the other components of the naturally occurring organism or virus, for example, the cell or viral structural components or other polypeptides or nucleic acids commonly found associated with the polypeptide. In some embodiments an “isolated” polypeptide is enriched by at least about 10-fold, about 100-fold, about 1000-fold, about 10,000-fold or more as compared with the starting material.

As used herein, by “isolate” or “purify” (or grammatical equivalents) a virus vector, it is meant that the virus vector is at least partially separated from at least some of the other components in the starting material. In some embodiments an “isolated” or “purified” virus vector is enriched by at least about 10-fold, about 100-fold, about 1000-fold, about 10,000-fold or more as compared with the starting material.

A “therapeutic” polypeptide or protein is one that can alleviate, reduce, prevent, delay and/or stabilize symptoms that result from an absence or defect in a protein in a cell or subject and/or is a polypeptide that otherwise confers a benefit to a subject, e.g., anti-cancer effects or improvement in transplant survivability.

By the terms “treat,” “treating” or “treatment of” (and grammatical variations thereof) it is meant that the severity of the subject's condition is reduced, at least partially improved or stabilized and/or that some alleviation, mitigation, decrease or stabilization in at least one clinical symptom is achieved and/or there is a delay in the progression of the disease or disorder.

The terms “prevent,” “preventing” and “prevention” (and grammatical variations thereof) refer to prevention and/or delay of the onset of a disease, disorder and/or a clinical symptom(s) in a subject and/or a reduction in the severity of the onset of the disease, disorder and/or clinical symptom(s) relative to what would occur in the absence of the compositions and/or methods described herein. The prevention can be complete, e.g., the total absence of the disease, disorder and/or clinical symptom(s). The prevention can also be partial, such that the occurrence of the disease, disorder and/or clinical symptom(s) in the subject and/or the severity of onset is less than what would occur in the absence of the compositions and/or methods described herein.

“Therapeutically effective amount” as used herein refers to an amount that, when administered to a subject for treating a disease, or at least one of the clinical symptoms of a disease, is sufficient to affect such treatment of the disease or symptom thereof. The “therapeutically effective amount” may vary depending, for example, on the disease and/or symptoms of the disease, severity of the disease and/or symptoms of the disease or disorder, the age, weight, and/or health of the patient to be treated, and the judgment of the prescribing physician. An appropriate amount in any given instance may be ascertained by those skilled in the art or capable of determination by routine experimentation.

As used herein, the terms “virus vector,” “vector” or “gene delivery vector” refer to a virus (e.g., AAV) particle that functions as a nucleic acid delivery vehicle, and which comprises the vector genome (e.g., viral DNA [vDNA]) packaged within a virion. Alternatively, in some contexts, the term “vector” may be used to refer to the vector genome/vDNA alone. In some embodiments, the vector genome (e.g., the AAV vector genome) may be comprised in a “cargo nucleic acid.” In some embodiments, the vector genome is self-complementary (i.e., double stranded). In some embodiments, the vector genome is not self-complimentary (i.e., single stranded).

A “rAAV vector genome” or “rAAV genome” is an AAV genome (i.e., vDNA) that comprises one or more heterologous nucleic acid sequences. rAAV vectors generally require only the inverted terminal repeat(s) (ITR(s)) in cis to generate virus. All other viral sequences are dispensable and may be supplied in trans (Muzyczka, (1992) Curr. Topics Microbiol. Immunol. 158:97). Typically, the rAAV vector genome will only retain the one or two ITR sequences so as to maximize the size of the transgene that can be efficiently packaged by the vector. The structural and non-structural protein coding sequences may be provided in trans (e.g., from a vector, such as a plasmid, or by stably integrating the sequences into a packaging cell). In some embodiments, the rAAV vector genome comprises at least one ITR sequence (e.g., AAV ITR sequence), optionally two ITRs (e.g., two AAV ITRs), which typically will be at the 5′ and 3′ ends of the vector genome (i.e., the 5′ ITR and the 3′ ITR) and flank the heterologous nucleic acid, but need not be contiguous thereto.

The term “inverted terminal repeat” or “ITR” includes any viral terminal repeat or synthetic sequence that forms a hairpin structure and functions as an inverted terminal repeat (i.e., mediates the desired functions such as replication, virus packaging, integration and/or provirus rescue, and the like). The ITR can be an AAV ITR or a non-AAV ITR. For example, a non-AAV ITR sequence such as those of other parvoviruses (e.g., canine parvovirus (CPV), mouse parvovirus (MVM), human parvovirus B-19) or any other suitable virus sequence (e.g., the SV40 hairpin that serves as the origin of SV40 replication) can be used as an ITR, which can further be modified by truncation, substitution, deletion, insertion and/or addition. Further, the ITR can be partially or completely synthetic, such as the “double-D sequence” as described in U.S. Pat. No. 5,478,745.

An “AAV inverted terminal repeat” or “AAV ITR” may be from any AAV, including but not limited to serotypes 1, 2, 3, 4, 5, 6, 7, 8, 9, 10, 11, 12, 13 or any other AAV now known or later discovered (see, e.g., Table 2). An AAV inverted terminal repeat need not have the native terminal repeat sequence (e.g., a native AAV ITR sequence may be altered by insertion, deletion, truncation and/or missense mutations), as long as the terminal repeat mediates the desired functions, e.g., replication, virus packaging, integration, and/or provirus rescue, and the like.

The virus vectors described herein can further be “targeted” virus vectors (e.g., having a directed tropism) and/or “hybrid” virus vectors (i.e., in which the viral ITRs and viral capsid are from different viruses) as described in international patent publication WO00/28004 and Chao et al, (2000) Molecular Therapy 2:619. In some embodiments, the virus vectors are targeted to a cell and/or tissue of the CNS.

The virus vectors described herein can further be duplexed virus particles as described in international patent publication WO 01/92551 (the disclosure of which is incorporated herein by reference in its entirety). Thus, in some embodiments, double stranded (duplex) genomes can be packaged into the virus capsids described herein. Further, the viral capsid or genomic elements can contain other modifications, including insertions, deletions and/or substitutions.

As used herein, the term “amino acid” encompasses any naturally occurring amino acid, modified forms thereof, and synthetic amino acids. Naturally occurring, levorotatory (L-) amino acids are shown in Table 3.

TABLE 3
Amino acid residues and abbreviations.

| | Abbreviation | |
|---|---|---|
| Amino Acid Residue | Three-Letter Code | One-Letter Code |
| Alanine | Ala | A |
| Arginine | Arg | R |
| Asparagine | Asn | N |
| Aspartic acid (Aspartate) | Asp | D |
| Cysteine | Cys | C |
| Glutamine | Gln | Q |
| Glutamic acid (Glutamate) | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

Alternatively, the amino acid can be a modified amino acid residue (nonlimiting examples are shown in Table 4) and/or can be an amino acid that is modified by post-translation modification (e.g., acetylation, amidation, formylation, hydroxylation, methylation, phosphorylation or sulfatation). Methods of chemically modifying amino acids are known in the art (see, e.g., Greg T. Hermanson, Bioconjugate Techniques, 1^{st} edition, Academic Press, 1996).

TABLE 4
Modified Amino Acid Residues

| | |
|---|---|
| Modified Amino Acid Residue | Abbreviation |

Amino Acid Residue Derivatives

| | |
|---|---|
| 2-Aminoadipic acid | Aad |
| 3-Aminoadipic acid | bAad |
| beta-Alanine, beta-Aminoproprionic acid | bAla |
| 2-Aminobutyric acid | Abu |
| 4-Aminobutyric acid, Piperidinic acid | 4Abu |
| 6-Aminocaproic acid | Acp |
| 2-Aminoheptanoic acid | Ahe |
| 2-Aminoisobutyric acid | Aib |
| 3-Aminoisobutyric acid | bAib |
| 2-Aminopimelic acid | Apm |
| t-butylalanine | t-BuA |
| Citrulline | Cit |
| Cyclohexylalanine | Cha |
| 2,4-Diaminobutyric acid | Dbu |
| Desmosine | Des |
| 2,21-Diaminopimelic acid | Dpm |
| 2,3-Diaminoproprionic acid | Dpr |
| N-Ethylglycine | EtGly |
| N-Ethylasparagine | EtAsn |
| Homoarginine | hArg |
| Homocysteine | hCys |
| Homoserine | hSer |
| Hydroxylysine | Hyl |
| Allo-Hydroxylysine | aHyl |
| 3-Hydroxyproline | 3Hyp |
| 4-Hydroxyproline | 4Hyp |
| Isodesmosine | Ide |
| allo-Isoleucine | alle |
| Methionine sulfoxide | MSO |
| N-Methylglycine, sarcosine | MeGly |
| N-Methyl isoleucine | Melle |
| 6-N-Methyllysine | MeLys |
| N-Methylvaline | MeVal |
| 2-Naphthylalanine | 2-Nal |
| Norvaline | Nva |
| Norleucine | Nle |
| Ornithine | Orn |
| 4-Chlorophenylalanine | Phe(4-C1) |
| 2-Fluorophenylalanine | Phe(2-F) |
| 3-Fluorophenylalanine | Phe(3-F) |
| 4-Fluorophenylalanine | Phe(4-F) |
| Phenylglycine | Phg |
| Beta-2-thienylalanine | Thi |

Further, the non-naturally occurring amino acid can be an “unnatural” amino acid (as described by Wang et al., Annu Rev Biophys Biomol Struct. 35-225-49 (2006)). These unnatural amino acids can advantageously be used to chemically link molecules of interest to the AAV capsid protein.

### Modified AAV Capsid Proteins and AAV Vectors Comprising the Same

### AAV Vectors

Additionally provided herein are adeno-associated virus (AAV) vectors comprising (i) a recombinant capsid protein and (ii) a cargo nucleic acid encapsidated by the capsid protein. In some embodiments, the recombinant capsid proteins (VP1, VP2 and/or VP3) may comprise a peptide in their amino acid sequence that does not occur in any native AAV capsid sequence. The inventors have demonstrated that capsid proteins comprising the peptides described herein can confer one or more desirable properties to virus vectors including, without limitation, the ability to evade neutralizing antibodies. Thus, AAV vectors described herein address the limitations associated with conventional AAV vectors.

Accordingly, in some embodiments, the present disclosure provides adeno-associated virus (AAV) vectors comprising (i) a recombinant capsid protein and (ii) a cargo nucleic acid encapsidated by the capsid protein; wherein the capsid protein comprises a peptide having the sequence of any one of SEQ ID NO: 12-20. In some embodiments, the cargo nucleic acid comprises 5′ and 3′ AAV inverted terminal repeats. In some embodiments, the cargo nucleic acid comprises a transgene. In some embodiments, the cargo nucleic acid is double stranded. In some embodiments, the cargo nucleic acid is single stranded. In some embodiments, the transgene encodes a therapeutic protein or RNA. In some embodiments, the recombinant capsid protein has at least 90%, at least 95%, at least 96%, at least 97%, at least 98%, or at least 99% sequence identity to the native sequence of the AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV or avian AAV capsid. In some embodiments, the recombinant capsid protein has at least 90% sequence identity to the native sequence of the AAV9 capsid.

In some embodiments, the peptide is located at the amino acid positions corresponding to amino acids 451-458 of the native AAV9 capsid, or the equivalent amino acid residues in AAV1, AAV2, AAV3, AAV4, AAV5, AAV6, AAV7, AAV8, AAV10, AAV11, AAV12, AAVrh.8, AAVrh.10, AAVrh32.33, AAVrh74, bovine AAV or avian AAV, and the peptide is selected from any one of SEQ ID NO: 12-18. In some embodiments, the peptide is located at the amino acid positions corresponding to amino acids 587-594 of the native AAV9 capsid, or the equivalent amino acid residues in AAV1, AAV2, AAV3, AAV4, AAV5, AAV6, AAV7, AAV8, AAV10, AAV11, AAV12, AAVrh.8, AAVrh.10, AAVrh32.33, AAVrh74, bovine AAV or avian AAV, and the peptide is selected from SEQ ID NO: 19 or 20.

In some embodiments, a recombinant capsid protein comprises a) a first peptide having a sequence of any one of SEQ ID NO: 12-18; and b) a second peptide having a sequence of any one of SEQ ID NO: 19-20. In some embodiments, the first peptide is at amino acid positions 451-458, and the second peptide is at amino acids 587-594, wherein the amino acid numbering is based on the native AAV9 capsid, or the equivalent amino acid residues in AAV1, AAV2, AAV3, AAV4, AAV5, AAV6, AAV7, AAV8, AAV10, AAV11, AAV12, AAVrh.8, AAVrh.10, AAVrh32.33, AAVrh74, bovine AAV or avian AAV.

In some embodiments, the peptide inhibits binding of at least one antibody to the capsid protein. In some embodiments, the peptide inhibits neutralization of infectivity of the AAV vector by the antibody.

In some embodiments, the peptide selectively binds to a receptor expressed on the surface of a cell in the central nervous system (CNS). In some embodiments, the cell is in the premotor cortex, the thalamus, the cerebellar cortex, the dentate nucleus, the spinal cord, or the dorsal root ganglion. In some embodiments, the peptide selectively binds to a receptor expressed on the surface of a cell in the heart.

In some embodiments, an adeno-associated virus (AAV) vector comprises (i) a mutant AAV9 capsid protein and (ii) a cargo nucleic acid encapsidated by the capsid protein, wherein the capsid protein comprises a peptide having the sequence X¹-X²-X³—X⁴-X⁵-X⁶-X⁷-X⁸ (SEQ ID NO: 158) at amino acids 451-458 of the native AAV9 capsid protein sequence, wherein the peptide does not occur in the native AAV9 capsid protein sequence. In some embodiments, X¹ is not I, X² is not N, X³ is not G, X⁴ is not S, X⁵ is not G, X⁶ is not Q, X⁷ is not N, and/or X⁸ is not Q. In some embodiments, X¹ is S, F, Q, G, K, or R. In some embodiments, X² is C, G, R, D, T, or Q. In some embodiments, X³ is Q, V, G, Y, R, F, or D. In some embodiments, X⁴ is P, Q, A, or R. In some embodiments, X⁵ is T, N, A, P, or I. In some embodiments, X⁶ is V, Q, A, or I. In some embodiments, X⁷ is M, P, R, Q, or N. In some embodiments, X⁸ is N, L, F, E, H, or A. In some embodiments, X¹ is S, X² is C, X³ is Q, X⁴ is P, X⁵ is T, X⁶ is V, X⁷ is M, and X⁸ is N. In some embodiments, X¹ is F, X² is G, X³ is V, X⁴ is P, X⁵ is N, X⁶ is Q, X⁷ is P, and X⁸ is L. In some embodiments, X¹ is Q, X² is R, X³ is G, X⁴ is Q, X⁵ is A, X⁶ is A, X⁷ is P, and X⁸ is F. In some embodiments, X¹ is G, X² is D, X³ is Y, X⁴ is A, X⁵ is P, X⁶ is I, X⁷ is R, and X⁸ is E. In some embodiments, X¹ is K, X² is T, X³ is R, X⁴ is R, X⁵ is I, X⁶ is V, X⁷ is Q, and X⁸ is H. In some embodiments, X¹ is F, X² is G, X³ is F, X⁴ is P, X⁵ is N, X⁶ is Q, X⁷ is P, and X⁸ is L. In some embodiments, X¹ is R, X² is Q, X³ is D, X⁴ is Q, X⁵ is P, X⁶ is I, X⁷ is N, and X⁸ is A.

In some embodiments, an adeno-associated virus (AAV) vector comprises (i) a mutant AAV9 capsid protein and (ii) a cargo nucleic acid encapsidated by the capsid protein, wherein the capsid protein comprises a peptide having the sequence X¹-X²-X³—X⁴-X⁵-X⁶-X⁷-X⁸ (SEQ ID NO: 158) at amino acids 587-594 of the native AAV9 capsid protein sequence, wherein the peptide does not occur in the native AAV9 capsid protein sequence. In some embodiments, X¹ is not A, X² is not Q, X³ is not A, X⁴ is not Q, X⁵ is not A, X⁶ is not Q, X⁷ is not T, and/or X⁸ is not G. In some embodiments, X¹ is S. In some embodiments, X² is K or T. In some embodiments, X³ is V. In some embodiments, X⁴ is E or D. In some embodiments, X⁵ is S. In some embodiments, X⁶ is W or I. In some embodiments, X⁷ is T or A. In some embodiments, X⁸ is E or I. In some embodiments, X¹ is S, X² is K, X³ is V, X⁴ is E, X⁵ is S, X⁶ is W, X⁷ is T, and X⁸ is E. In some embodiments, X¹ is S, X² is T, X³ is V, X⁴ is D, X⁵ is S, X⁶ is I, X⁷ is A, and X⁸ is I.

In some embodiments, an adeno-associated virus (AAV) vector comprises (i) a recombinant capsid protein and (ii) a cargo nucleic acid encapsidated by the capsid protein, wherein the capsid protein comprises an amino acid sequence that is at least 95%, at least 96%, at least 97%, at least 98%, or at least 99% identical to any one of SEQ ID NO: 165-187. In some embodiments, the capsid protein comprises the amino acid sequence of any one of SEQ ID NO: 165-187. In some embodiments, the capsid protein comprises the amino acid sequence of SEQ ID NO: 175. In some embodiments, the capsid protein comprises the amino acid sequence of SEQ ID NO: 180.

In some embodiments, an AAV vector selectively delivers the cargo nucleic acid to a cell or tissue of the central nervous system. In some embodiments, the tissue of the central nervous system is the premotor cortex, the thalamus, the cerebellar cortex, the dentate nucleus, the spinal cord, or the dorsal root ganglion. In some embodiments, the AAV vector delivers the cargo nucleic acid to the brain, but does not deliver the AAV vector to the heart. In some embodiments, the AAV vector delivers the cargo nucleic acid to the brain and to the heart. In some embodiments, delivery of the cargo nucleic acid is greater to the brain than to the heart. In some embodiments, delivery of the cargo nucleic acid is approximately equal in the brain and in the heart.

### AAV Capsid Proteins

In some embodiments, the disclosure provides an adeno-associated virus (AAV) capsid protein comprising one or more amino acid modifications (e.g., substitutions and/or deletions) compared to a native AAV capsid protein, wherein the one or more modifications modify one or more antigenic sites on the AAV capsid protein. The modification of the one or more antigenic sites results in inhibition of binding by an antibody to the one or more antigenic sites and/or inhibition of neutralization of infectivity of a virus particle comprising the AAV capsid protein. The one or more amino acid modifications (e.g., substitutions and/or deletions) can be in one or more antigenic footprints identified by peptide epitope mapping and/or cryo-electron microscopy studies of AAV-antibody complexes containing AAV capsid proteins. In some embodiments, the one or more antigenic sites are common antigenic motifs or CAMs as described in WO 2017/058892, which is incorporated herein by reference in its entirety. In some embodiments, the antigenic sites are in a variable region (VR) of the AAV capsid protein, such as VR-I, VR-II, VR-III, VR-IV, VR-V, VR-VI, VR-VII, VR-VIII, VR-IX. In some embodiments, one or more antigenic sites is in the HI loop of the AAV capsid protein.

In some embodiments, an AAV1, AAV2, AAV3, AAV4, AAV5, AAV6, AAV7, AAV8, AAV9, AAVrh8, AAVrh10, AAV10, AAV11, AAV12, AAVrh32.22, bovine AAV, or Avian AAV capsid protein comprises an amino acid modification (e.g., a substitution or deletion) in one or more of the regions identified in Table 5, below.

TABLE 5
Exemplary antigenic or other regions on various AAV capsids that may be partially or fully
substituted/replaced. Respective VP1 numbering of residues in the native AAV capsid
sequence is shown.

| AAV1 | | AAV2 | | AAV3 | | AAV4 | | AAV5 | |
|---|---|---|---|---|---|---|---|---|---|
| Sequence | | Sequence | | Sequence | | Sequence | | Sequence | |
| (amino | SEQ | (amino | SEQ | (amino | SEQ | (amino | SEQ | (amino | SEQ |
| acid | ID | acid | ID | acid | ID | acid | ID | acid | ID |
| numbers) | NO | numbers) | NO | numbers) | NO | numbers) | NO | numbers) | NO |
| SASTGAS | 2591 | SQSGAS | 2601 | SQSGAS | 2611 | RLGESLQS | 2621 | EIKSGSVDGS | 2631 |
| (262-268) | | (262-267) | | (262-267) | | (253-260) | | (249-258) | |
| VFMIPQY | 2592 | VFMVPQY | 2602 | VFMVPQ | 2612 | VFMVPQ | 2622 | VFTLPQY | 2632 |
| GYL (370- | | GYL (369- | | YGYL | | YGYC | | GYA (360- | |
| 379) | | 378) | | (369-378) | | (360-369) | | 369 | |
| NQSGSA | 2593 | TPSGTTT | 2603 | TTSGTTN | 2613 | GTTLNA | 2623 | STNNTGG | 2633 |
| QNK | | QS (450- | | QS (451- | | GTA | | VQ (440- | |
| (451-459) | | 458) | | 459) | | (445-453) | | 448) | |
| SV (472- | 2594 | RD (471- | 2604 | SL (472- | 2614 | SN (466- | 2624 | AN (458- | 2634 |
| 473) | | 472) | | 473) | | 467 | | 459) | |
| KTDNNN | 2595 | SADNNNS | 2605 | ANDNNN | 2615 | ANQNYKI | 2625 | SGVNRAS | 2635 |
| SN (493- | | E (492- | | SN (493- | | PATGS | | (479-485) | |
| 500) | | 499) | | 500) | | (487-498) | | | |
| KDDEDK | 2596 | KDDEEKF | 2606 | KDDEEK | 2616 | GPADSK | 2626 | LQGSNTY | 2636 |
| F (528- | | (527-533) | | F (528- | | F (527- | | (515-521) | |
| 534) | | | | 534) | | 533 | | | |
| SAGASN | 2597 | GSEKTN | 2607 | GTTASN | 2617 | QNGNTA | 2627 | ANPGTTA | 2637 |
| (547-552) | | (546-551) | | (547-552) | | (545-560) | | T (534-541) | |
| STDPATG | 2598 | NRQAATA | 2608 | NTAPTTG | 2618 | SNLPTV | 2628 | TTAPATG | 2638 |
| DVH (588- | | DVN (587- | | TVN (588- | | DRLT | | TYN (577- | |
| 597 | | 596) | | 597) | | (583-595) | | 586) | |
| AN (709- | 2599 | VN (708- | 2609 | VN (709- | 2619 | NS (707- | 2629 | QF (697- | 2639 |
| 710) | | 709) | | 710 | | 708) | | 698 | |
| DNNGLY | 2600 | DTNGVYS | 2610 | DTNGVY | 2620 | DAAGKY | 2630 | DSTGEYR | 2640 |
| T (716- | | (715-721) | | S (716- | | T (714- | | (704-710) | |
| 722) | | | | 722) | | 720) | | | |
| AAV6 | | AAV7 | | AAV8 | | AAV9 | | AAVrh8 | |
| (amino | SEQ | (amino | SEQ | (amino | SEQ | (amino | SEQ | (amino | SEQ |
| acid | ID | acid | ID | acid | ID | acid | ID | acid | ID |
| numbers | NO | numbers) | NO | numbers) | NO | numbers | NO | numbers) | NO |
| SASTGAS | 2641 | SETAGST | 2651 | NGTSGG | 2661 | NSTSGG | 2671 | NGTSGGS | 2681 |
| (262-268) | | (263-269) | | AT (263- | | SS (262- | | T (262-269) | |
| | | | | 270) | | 269) | | | |
| VFMIPQY | 2642 | VFMIPQY | 2652 | VFMIPQY | 2662 | VFMIPQY | 2672 | VFMVPQY | 2682 |
| GYL (370- | | GYL (371- | | GYL (372- | | GYL | | GYL (371-380) | |
| 379 | | 380) | | 381) | | (371-380) | | | |
| NQSGSA | 2643 | NPGGTA | 2653 | TTGGTA | 2663 | INGSGQ | 2673 | QTTGTGGTQ | 2683 |
| QNK | | GNR (453- | | NTQ | | NQQ | | (451-459) | |
| (451-459) | | 461) | | (453-461) | | (451-459) | | | |
| SV (472- | 2644 | AN (474- | 2654 | AN (474- | 2664 | AV (472- | 2674 | AN (472-473) | 2684 |
| 473) | | 475) | | 475) | | 473) | | | |
| KTDNNN | 2645 | LDQNNNS | 2655 | TGQNNN | 2665 | VTQNNN | 2675 | TNQNNNS | 2685 |
| SN (493- | | N (495- | | SN (495- | | SE (493- | | N (493- | |
| 500) | | 502) | | 502) | | 500) | | 500) | |
| KDDKDK | 2646 | KDDEDRF | 2656 | KDDEER | 2666 | KEGEDR | 2676 | KDDDDRF | 2686 |
| F (528- | | (530-536) | | F (530- | | F (528- | | (528-534) | |
| 534) | | | | 536) | | 534) | | | |
| SAGASN | 2647 | GATNKT | 2657 | NAARDN | 2667 | GTGRDN | 2677 | GAGNDG | 2687 |
| (547-552) | | (549-554) | | (549-554) | | (547-552) | | (547-552) | |
| STDPATG | 2648 | NTAAQTQ | 2658 | NTAPQIG | 2668 | QAQAQT | 2678 | NTQAQTG | 2688 |
| DVH (588- | | VVN (589- | | TVNS | | GWVQ | | LVH (588-597) | |
| 897) | | 598) | | (590-600) | | (588-597) | | | |
| AN (709- | 2649 | TG (710- | 2659 | TS (711- | 2669 | NN (709- | 2679 | TN (709-710) | 2689 |
| 710) | | 711) | | 712) | | 710) | | | |
| DNNGLY | 2650 | DSQGVY | 2660 | NTEGVY | 2670 | NTEGVY | 2680 | NTEGVYS | 2690 |
| T (716- | | S (717- | | S (718- | | S (716- | | (716-722) | |
| 722) | | 723) | | 724) | | 722) | | | |
| AAVrh10 | | AAV10 | | AAV11 | | AAV12 | | | |
| (amino | SEQ | (amino | SEQ | (amino | SEQ | (amino | SEQ | AAVrh32.33 | SEQ |
| acid | ID | acid | ID | acid | ID | acid | ID | (amino acid | ID |
| numbers) | NO | numbers) | NO | numbers) | NO | numbers | NO | numbers) | NO |
| NGTSGGS | 2691 | NGTSGG | 2701 | RLGTTSS | 2711 | RIGTTAN | 2721 | RLGTTSNS | 2731 |
| T (263- | | ST (263- | | S (253- | | S (262- | | (253-260) | |
| 270 | | 270 | | 260) | | 269 | | | |
| VFMIPQY | 2692 | VFMIPQY | 2702 | VFMVPQ | 2712 | VFMVPQ | 2722 | VFMVPQYG | 2732 |
| GYL (372- | | GYL (372- | | YGYC | | YGYC | | YC (360-369) | |
| 381) | | 381) | | (360-369) | | (369-378) | | | |
| STGGTAG | 2693 | STGGTQ | 2703 | GETLNQ | 2713 | GNSLNQ | 2723 | GETLNQGN | 2733 |
| TQ (453- | | GTQ (453- | | GNA | | GTA | | A (444-452) | |
| 461) | | 461) | | (444-452) | | (453-461) | | | |
| SA (474- | 2694 | SA (474- | 2704 | AF (465- | 2714 | AY (474- | 2724 | AF (465-466) | 2734 |
| 475) | | 475) | | 466) | | 475 | | | |
| LSQNNNS | 2695 | LSQNNNS | 2705 | ASQNYKI | 2715 | ANQNYKI | 2725 | ASQNYKIPA | 2735 |
| N (495- | | N (495- | | PASGG | | PASGG | | SGG (486- | |
| 502) | | 502) | | (486-497) | | (495-506) | | 497 | |
| KDDEERF | 2696 | KDDEERF | 2706 | GPSDGD | 2716 | GAGDSD | 2726 | GPSDGDF | 2736 |
| (530-536) | | (530-536) | | F (526-532) | | F (535-541) | | (526-532) | |
| GAGKDN | 2697 | GAGRDN | 2707 | VTGNTT | 2717 | PSGNTT | 2727 | VTGNTT | 2737 |
| (549-554) | | (549-554) | | (544-549) | | (553-558) | | (544-549) | |
| NAAPIVG | 2698 | NTGPIVG | 2708 | TTAPITG | 2718 | TTAPHIA | 2728 | TTAPITGNV | 2738 |
| AVN (590- | | NVN (590- | | NVT (585- | | NLD | | T (585-594) | |
| 599) | | 599 | | 594) | | (594-503) | | | |
| TN (711- | 2699 | TN (711- | 2709 | SS (706- | 2719 | NS (715- | 2729 | SS (706-707) | 2739 |
| 712 | | 712) | | 707) | | 716) | | | |
| NTDGTYS | 2700 | NTEGTYS | 2710 | DTTGKYT | 2720 | DNAGNY | 2730 | DTTGKYT | 2740 |
| (718-724) | | (718-724) | | (713-719) | | H (722-728) | | (713-719) | |

| Bovine AAV (amino acid numbers) | SEQ ID NO | Avian AAV (amino acid numbers) | SEQ ID NO |
|---|---|---|---|
| RLGSSNAS (255-262) | 2741 | RIQGPSGG (265-272) | 2751 |
| VFMVPQYGYC (362-371) | 2742 | IYTIPQYGYC (375-384) | 2752 |
| GGTLNQGNS (447-455) | 2743 | VSQAGSSGR (454-462) | 2753 |
| SG (468-469) | 2744 | AA (475-476) | 2754 |
| ASQNYKIPQGRN (489-500) | 2745 | ASNITKNNVFSV (496-507) | 2755 |
| ANDATDF (529-535) | 2746 | FSGEPDR (533-539) | 2756 |
| ITGNTT (547-552) | 2747 | VYDQTTAT (552-559) | 2757 |
| TTVPTVDDVD (588-597) | 2748 | VTPGTRAAVN (595-604) | 2758 |
| DS (709-710) | 2749 | AD (716-717) | 2759 |
| DNAGAYK (716-722) | 2750 | SDTGSYS (723-729) | 2760 |

In some embodiments, the amino acid substitution replaces any eight amino acids in an AAV capsid protein from any one of the following serotypes: AAV1, AAV2, AAV3, AAV4, AAV5, AAV6, AAV7, AAV8, AAV9, AAVrh8, AAVrh10, AAV10, AAV11, AAV12, AAVrh32.22, bovine AAV, or Avian AAV. For example, the amino acid substitution may replace the following amino acids (VP1 numbering): 355-362, 363-370, 371-378, 379-386, 387-394, 395-402, 403-410, 411-418, 419-426, 427-434, 435-442, 443-450, 451-458, 459-466, 467-474, 475-482, 483-490, 491-498, 499-506, 507-514, 515-522, 523-530, 531-538, 539-546, 547-554, 555-562, 563-570, 571-578, 579-586, 587-594, 595-602, 603-610, 611-618, 619-626, 627-634, 635-642, 643-650, 651-658, 659-666, 667-674, 675-682, 683-690, 691-698, 699-706, 707-714, 715-722 in any of the above-listed AAV serotypes.

In some embodiments, the amino acid substitution is selected from any one of SEQ ID NO: 19-20. In some embodiments, the amino acid substitution has at least 95%, at least 96%, at least 97%, at least 98%, or at least 99% sequence homology with any one of SEQ ID NO: 12-18. In some embodiments, the substitution is at the amino acids corresponding to amino acids 587-594 of the wildtype AAV9 capsid. In some embodiments, the substitution is at the amino acids corresponding to amino acids 587-594 of the wildtype AAV1 capsid. In some embodiments, the substitution is at the amino acids corresponding to amino acids 587-594 of the wildtype AAV6 capsid. In some embodiments, the substitution is at the amino acids corresponding to amino acids 589-596 of the wildtype AAV8 capsid. In some embodiments, the substitution is at the amino acids corresponding to amino acids 587-594 of the wildtype AAVrh8 capsid. In some embodiments, the substitution is at the amino acids corresponding to amino acids 589-596 of the wildtype AAVrh10 capsid.

In some embodiments, the amino acid substitution is selected from any one of SEQ ID NO: 18-20. In some embodiments, the amino acid substitution has at least 95%, at least 96%, at least 97%, at least 98%, or at least 99% sequence homology with any one of SEQ ID NO: 18-20. In some embodiments, the substitution is at the amino acids corresponding to amino acids 451-458 of the wildtype AAV9 capsid.

In some embodiments, an amino acid deletion comprises a deletion of at least one, at least two, at least three, at least four, at least five, at least six, at least seven, at least eight, at least nine, or at least ten amino acids compared to the wildtype capsid.

In some embodiments, an AAV capsid comprises one or more amino acid substitutions and one or more amino acid deletions. In some embodiments, a capsid comprises at least one amino acid substitution and at least one amino acid deletion. In some embodiments, a capsid comprises at least one amino acid substitution and at least one amino acid deletion, wherein the at least one amino acid substitution and the at least one amino acid deletion are immediately adjacent to one another in the capsid amino acid sequence.

In some embodiments, the capsid proteins are modified to produce an AAV capsid that, when present in an AAV virus particle or AAV virus vector, has a phenotype of selectively targeting the CNS (e.g., the brain, the spinal cord). In some embodiments, the capsid proteins are modified to produce an AAV capsid that, when present in an AAV virus particle or AAV virus vector, has a phenotype of evading neutralizing antibodies. The AAV virus particle or vector can also have a phenotype of enhanced or maintained transduction efficiency in addition to the phenotype of evading neutralizing antibodies and/or targeting the CNS.

In some embodiments, the one or more substitutions can introduce one or more sequences from a capsid protein of a first AAV serotype into the capsid protein of a second AAV serotype that is different from the first AAV serotype.

The base AAV capsid protein to which modifications are added can be a capsid protein of an AAV serotype selected from AAV1, AAV2, AAV3, AAV3B, AAV4, AAV5, AAV6, AAV7, AAV8, AAV9, AAV10, AAV11, AAV12, AAVrh.8, AAVrh.10, AAVrh.32.33, AAVrh74, bovine AAV, avian AAV or any other AAV now known or later identified. In some embodiments, the base AAV capsid protein is of the AAV9 serotype. In some embodiments, the base AAV capsid protein is chimeric. In some embodiments, the base AAV capsid protein is an AAV8/9 chimera.

Several examples of a modified AAV capsid protein are provided herein. In the following examples, the capsid protein can comprise the specific substitutions described and, in some embodiments, can comprise fewer or more substitutions than those described. As used herein, “substitution” may refer to a single amino acid substitution, or a substitution of more than one contiguous amino acid. For example in some embodiments, a capsid protein can comprise at least 1, 2, 3, 4, 5, 6, 7, 8, 9, 10, etc., single amino acid substitutions. In some embodiments, a capsid protein can comprise one or more substitutions of multiple contiguous amino acids, such as one or more substitutions of 2, 3, 4, 5, 6, 7, 8, 9, 10, 11, or 12 contiguous amino acids.

Furthermore, in some embodiments described herein wherein an amino acid residue is substituted by any amino acid residue other than the amino acid residue present in the wildtype or native amino acid sequence, the any other amino acid residue can be any natural or non-natural amino acid residue known in the art (see, e.g., Tables 2 and 3). In some embodiments, the substitution can be a conservative substitution and in some embodiments, the substitution can be a nonconservative substitution. In some embodiments, an AAV capsid protein comprises one or more amino acid substitutions, wherein the amino acid substitutions are each individually selected from SEQ ID NO: 12-18 as shown in Table 6.1.

TABLE 6.1
AMINO ACID SUBSTITUTIONS

| Amino Acid Substitution | SEQ ID NO. |
|---|---|
| SCQPTVMN | 12 |
| FGVPNQPL | 13 |
| QRGQAAPF | 14 |
| GDYAPIRE | 15 |
| KTRRIVQH | 16 |
| FGFPNQPL | 17 |
| RQDQPINA | 18 |

In some embodiments, an AAV capsid protein comprises one or more amino acid substitutions, wherein the amino acid substitutions are each selected from SEQ ID NO: 19-20 as shown in Table 6.2.

TABLE 6.2
AMINO ACID SUBSTITUTIONS

| Amino Acid Substitution | SEQ ID NO. |
|---|---|
| SKVESWTE | 19 |
| STVDSIAI | 20 |

In some embodiments, an AAV capsid protein may comprise a first substitution selected from the sequences listed in Table 6.1 and a second substitution selected from the sequences listed in Table 6.2. In some embodiments, an AAV capsid protein may comprise a first substitution, a second substitution as shown in Tables 6.3 and 6.4.

TABLE 6.3
COMBINATIONS OF AMINO ACID SUBSTITUTIONS

| First Substitution | Second Substitution |
|---|---|
| (SEQ ID NO) | (SEQ ID NO) |
| 12, 13 , 14 , 15 , 16, 17, | 19 or 20 |
| or 18 | |

TABLE 6.4
COMBINATIONS OF AMINO ACID SUBSTITUTIONS

| First Substitution | Second Substitution |
|---|---|
| (SEQ ID NO) | (SEQ ID NO) |
| 12 | 19 |
| 12 | 20 |
| 13 | 19 |
| 13 | 20 |
| 14 | 19 |
| 14 | 20 |
| 15 | 19 |
| 15 | 20 |
| 16 | 19 |
| 16 | 20 |
| 17 | 19 |
| 17 | 20 |
| 18 | 19 |
| 18 | 20 |

In some embodiments, an AAV capsid protein comprises an amino acid modification (e.g., substitution and/or deletion), wherein the amino acid modification modifies one or more surface-exposed regions, such as an antigenic region, on the AAV capsid protein.

In some embodiments, an AAV capsid protein comprises one or more amino acid substitutions, wherein at least one of the amino acid substitutions comprises one of SEQ ID NOs: 19-20. In some embodiments, the substitution replaces the amino acids corresponding to amino acids 587-594 of the wildtype AAV9 capsid.

In some embodiments, an AAV capsid protein comprises one or more amino acid substitutions, wherein at least one of the amino acid substitutions comprises one of SEQ ID NOs: 12-18. In some embodiments, the substitution replaces the amino acids corresponding to amino acids 451-458 of the wildtype AAV9 capsid.

In some embodiments, an AAV capsid protein comprises a substitution comprising a sequence of eight amino acids (X¹—X²-X³-X⁴-X⁵-X⁶-X⁷-X⁸) (SEQ ID NO: 158) that does not occur in the native capsid protein sequence. In some embodiments, X¹ is not I, X² is not N, X³ is not G, X⁴ is not S, X⁵ is not G, X⁶ is not Q, X⁷ is not N, and/or X⁸ is not Q. In some embodiments, X¹ is S, F, Q, G, K, or R. In some embodiments, X² is C, G, R, D, T, or Q. In some embodiments, X³ is Q, V, G, Y, R, F, or D. In some embodiments, X⁴ is P, Q, A, or R. In some embodiments, X⁵ is T, N, A, P, or I. In some embodiments, X⁶ is V, Q, A, or I. In some embodiments, X⁷ is M, P, R, Q, or N. In some embodiments, X⁸ is N, L, F, E, H, or A. In some embodiments, X¹ is S, X² is C, X³ is Q, X⁴ is P, X⁵ is T, X⁶ is V, X⁷ is M, and X⁸ is N. In some embodiments, X¹ is F, X² is G, X³ is V, X⁴ is P, X⁵ is N, X⁶ is Q, X⁷ is P, and X⁸ is L. In some embodiments, X¹ is Q, X² is R, X³ is G, X⁴ is Q, X⁵ is A, X⁶ is A, X⁷ is P, and X⁸ is F. In some embodiments, X¹ is G, X² is D, X³ is Y, X⁴ is A, X⁵ is P, X⁶ is I, X⁷ is R, and X⁸ is E. In some embodiments, X¹ is K, X² is T, X³ is R, X⁴ is R, X⁵ is I, X⁶ is V, X⁷ is Q, and X⁸ is H. In some embodiments, X¹ is F, X² is G, X³ is F, X⁴ is P, X⁵ is N, X⁶ is Q, X⁷ is P, and X⁸ is L. In some embodiments, X¹ is R, X² is Q, X³ is D, X⁴ is Q, X⁵ is P, X⁶ is I, X⁷ is N, and X⁸ is A.

In some embodiments, X¹ is not A, X² is not Q, X³ is not A, X⁴ is not Q, X⁵ is not A, X⁶ is not Q, X⁷ is not T, and/or X⁸ is not G. In some embodiments, X¹ is S. In some embodiments, X² is K or T. In some embodiments, X³ is V. In some embodiments, X⁴ is E or D. In some embodiments, X⁵ is S. In some embodiments, X⁶ is W or I. In some embodiments, X⁷ is T or A. In some embodiments, X⁸ is E or I. In some embodiments, X¹ is S, X² is K, X³ is V, X⁴ is E, X⁵ is S, X⁶ is W, X⁷ is T, and X⁸ is E. In some embodiments, X¹ is S, X² is T, X³ is V, X⁴ is D, X⁵ is S, X⁶ is I, X⁷ is A, and X⁸ is I.

In some embodiments, an AAV capsid protein comprises one or more amino acid deletions, wherein the amino acid deletion comprises a deletion of at least six or at least eight amino acids compared to the wildtype AAV capsid. In some embodiments, an AAV capsid protein comprises a deletion of eight consecutive amino acids compared to the native capsid protein sequence. In some embodiments, an AAV capsid protein comprises a deletion of six consecutive amino acids compared to the native capsid protein sequence.

In some embodiments, an AAV capsid protein comprises the sequence LSKTQTLK (SEQ ID NO: 1374) or the sequence LSKTDPQTLK (SEQ ID NO: 1375). In some embodiments, the AAV capsid protein comprising SEQ ID NO: 1374 or 1375 is of a serotype selected from AAV1, AAV2, AAV3, AAV4, AAV5, AAV6, AAV7, AAV8, AAV9, AAV10, AAV11, AAV12, AAVrh8, AAVrh10, AAVrh32.33, AAVrh74, Avian AAV and Bovine AAV.

In some embodiments, an AAV capsid protein comprises a first substitution comprising a sequence selected from SEQ ID NO: 12-18; and a second substitution comprising a sequence selected from SEQ ID NO: 19-20.

In some embodiments, an AAV capsid protein comprises an amino acid deletion and a substitution, wherein the substitution comprises a sequence selected from SEQ ID NO: 12-20.

In some embodiments, a recombinant capsid protein has a sequence that is at least 90%, at least 95%, at least 96%, at least 97%, at least 98%, at least 99% or 100% identical to SEQ ID NO: 9 (AAV9) and comprises one or more of the following amino acid substitutions: 1451S, 1451F, 1451Q, 1451G, 1451K, 1451R, N452C, N452G, N452R, N452D, N452T, N452Q, G453Q, G453V, G453Y, G453R, G453F, G453D, S454P, S454Q, S454A, S454R, G455T, G455N, G455A, G455P, G4551, Q456V, Q456A, Q4561, N457M, N457P, N457R, N457Q, Q458N, Q458L, Q458F, Q458E, Q458H, Q458A, A587S, Q588K, Q588T, A589V, Q590E, Q590D, A591 S, Q592W, Q5921, T593A, G594E, G5941.

Any of the AAV capsids described herein may further comprise a modification (e.g., a substitution or a deletion) in the HI loop. The HI loop is a prominent domain on the AAV capsid surface, between β strands βH and βI, that extends from each viral protein (VP) subunit overlapping the neighboring fivefold VP. In some embodiments, an AAV capsid comprises one, two, three, four, five, six, seven, or eight amino acid substitutions in the HI loop. In some embodiments, the AAV capsid comprises one or more of the following substitutions in the HI loop: P661R, T662S, Q666G, S667D, wherein the numbering corresponds to the wildtype AAV8 capsid (SEQ ID NO: 8). In some embodiments, the AAV capsid comprises one or more of the following substitutions in the HI loop: P659R, T660S, A661T, K664G, wherein the numbering corresponds to the wildtype AAV9 capsid (SEQ ID NO: 9).

In some embodiments, an AAV capsid protein comprises one, two, three, or four amino acid substitutions, wherein each substitution modifies a different antigenic site on the AAV capsid protein, and wherein at least one of the amino acid substitutions modifies the HI loop of the capsid protein.

In some embodiments, an AAV capsid protein comprises a first, a second, a third, and a fourth amino acid substitution. In some embodiments, at least one of the substitutions modifies the HI Loop of the capsid protein. In some embodiments, the AAV capsid comprises one or more of the following substitutions in the HI loop: P661R, T662S, Q666G, S667D, wherein the numbering corresponds to the wildtype AAV8 capsid (SEQ ID NO: 8); or P659R, T660S, A661T, K664G, wherein the numbering corresponds to the wildtype AAV9 capsid (SEQ ID NO: 9). In some embodiments, an AAV capsid protein comprises the amino acid sequence of any one of SEQ ID NO: 185-187. In some embodiments, an AAV capsid protein comprises an amino acid sequence sharing at least 90%, at least 95%, at least 96%, at least 97%, at least 98%, or at least 99% sequence identity to any one of SEQ ID NO: 165-187.

Also provided herein is a nucleotide sequence, or an expression vector comprising the same that encodes one or more of the AAV capsid proteins described herein. The nucleotide sequence may be a DNA sequence or an RNA sequence. In some embodiments, cell comprises one or more nucleotide sequences or expression vectors described herein.

In some embodiments, an AAV capsid comprises an AAV capsid protein as described herein. Further provided herein is a viral vector comprising an AAV capsid as well as a composition comprising the AAV capsid protein, AAV capsid and/or viral vector in a pharmaceutically acceptable carrier.

In some embodiments, modification of one or more antigenic sites results in inhibition of binding by an antibody to the one or more antigenic sites. In some embodiments, modification of the one or more antigenic sites results in inhibition of neutralization of infectivity of a virus particle comprising the AAV capsid protein.

As described herein, the nucleic acid and amino acid sequences of the capsid proteins from a number of AAV are known in the art. Thus, the amino acids “corresponding” to amino acid positions of the native AAV capsid protein can be readily determined for any other AAV (e.g., by using sequence alignments).

The modified capsid proteins can be produced by modifying the capsid protein of any AAV now known or later discovered. Further, the base AAV capsid protein that is to be modified can be a naturally occurring AAV capsid protein (e.g., an AAV2, AAV3a or 3b, AAV4, AAV5, AAV6, AAV7, AAV8, AAV9, AAV10 or AAV11 capsid protein or any of the AAV shown in Table 2) but is not so limited. Those skilled in the art will understand that a variety of manipulations to the AAV capsid proteins are known in the art and the disclosure is not limited to modifications of naturally occurring AAV capsid proteins. For example, the capsid protein to be modified may already have alterations as compared with naturally occurring AAV (e.g., is derived from a naturally occurring AAV capsid protein, e.g., AAV2, AAV3a, AAV3b, AAV4, AAV5, AAV6, AAV7, AAV8, AAV9, AAV10, AAV11, AAV12 or any other AAV now known or later discovered). In some embodiments, the capsid protein may be a chimeric capsid protein. In some embodiments, the capsid protein may be an engineered AAV, such as AAV2i8, AAV2g9, AAV-LK03, AAV7m8, AAV Anc80, AAV PHP.B.

Thus, in some embodiments, the AAV capsid protein to be modified can be derived from a naturally occurring AAV but further comprises one or more foreign sequences (e.g., that are exogenous to the native virus) that are inserted and/or substituted into the capsid protein and/or has been altered by deletion of one or more amino acids.

Accordingly, when referring herein to a specific AAV capsid protein (e.g., an AAV2, AAV3, AAV4, AAV5, AAV6, AAV7, AAV8, AAV9, AAV10 or AAV11 capsid protein or a capsid protein from any of the AAV shown in Table 2, etc.), it is intended to encompass the native capsid protein as well as capsid proteins that have alterations other than the modifications described herein. Such alterations include substitutions, insertions and/or deletions. In some embodiments, the capsid protein comprises 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acids inserted therein (other than the insertions described herein) as compared with the native AAV capsid protein sequence. In some embodiments, the capsid protein comprises 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acid substitutions (other than the amino acid substitutions described herein) as compared with the native AAV capsid protein sequence, in some embodiments, the capsid protein comprises a deletion of 1, 2, 3, 4, 5, 6, 7, 8, 9, 10, 11, 12, 13, 14, 15, 16, 17, 18, 19 or 20, less than 20, less than 30, less than 40, less than 50, less than 60, or less than 70 amino acids as compared with the native AAV capsid protein sequence.

In some embodiments, the AAV capsid protein has an amino acid sequence that is at least about 90%, about 95%, about 97%, about 98% or about 99% similar or identical to a native AAV capsid protein sequence.

Methods of determining sequence similarity or identity between two or more amino acid sequences are known in the art. Sequence similarity or identity may be determined using standard techniques, including, but not limited to, the local sequence identity algorithm of Smith & Waterman, Adv. Appl. Math. 2, 482 (1981), by the sequence identity alignment algorithm of Needleman & Wunsch, J Mol. Biol. 48,443 (1970), by the search for similarity method of Pearson & Lipman, Proc. Natl. Acad. Sci. USA 85, 2444 (1988), by computerized implementations of these algorithms (GAP, BESTFIT, FASTA, and TFASTA in the Wisconsin Genetics Software Package, Genetics Computer Group, 575 Science Drive, Madison, WI), the Best Fit sequence program described by Devereux et al., Nucl. Acid Res. 12, 387-395 (1984), or by inspection.

Another suitable algorithm is the BLAST algorithm, described in Altschul et al., J Mol. Biol. 215, 403-410, (1990) and Karlin et al., Proc. Natl. Acad. Sci. USA 90, 5873-5787 (1993). A particularly useful BLAST program is the WU-BLAST-2 program which was obtained from Altschul et al., Methods in Enzymology, 266, 460-480 (1996); http://blast.wustl/edu/blast/README.html. WU-BLAST-2 uses several search parameters, which are optionally set to the default values. The parameters are dynamic values and are established by the program itself depending upon the composition of the particular sequence and composition of the particular database against which the sequence of interest is being searched; however, the values may be adjusted to increase sensitivity.

Further, an additional useful algorithm is gapped BLAST as reported by Altschul et al, (1997) Nucleic Acids Res. 25, 3389-3402.

In some embodiments, a virus capsid comprises a modified AAV capsid protein as described herein. In some embodiments, the virus capsid is a parvovirus capsid, which may further be an autonomous parvovirus capsid or a dependovirus capsid. Optionally, the virus capsid is an AAV capsid. In some embodiments, the AAV capsid is an AAV1, AAV2, AAV3a, AAV3b, AAV4, AAV5, AAV6, AAV7, AAV8, AAV9, AAV10, AAV11, AAV12, AAVrh8, AAVrh10, AAVrh32.33, bovine AAV capsid, avian AAV capsid or any other AAV now known or later identified. A nonlimiting list of AAV serotypes is shown in Table 2. An AAV capsid can be any AAV serotype listed in Table 2 or derived from any of the foregoing by one or more insertions, substitutions and/or deletions. Molecules that can be packaged by the modified virus capsid and transferred into a cell include cargo nucleic acids (e.g., heterologous DNA or RNA), polypeptides, small organic molecules, metals, or combinations of the same.

Heterologous molecules are defined as those that are not naturally found in an AAV infection, e.g., those not encoded by a wild-type AAV genome. Further, therapeutically useful molecules can be associated with the outside of the chimeric virus capsid for transfer of the molecules into host target cells. Such associated molecules can include DNA, RNA, small organic molecules, metals, carbohydrates, lipids and/or polypeptides. In some embodiments the therapeutically useful molecule is covalently linked (i.e., conjugated or chemically coupled) to the capsid proteins. Methods of covalently linking molecules are known by those skilled in the art.

The modified virus capsids also find use in raising antibodies against the novel capsid structures. As a further alternative, an exogenous amino acid sequence may be inserted into the modified virus capsid for antigen presentation to a cell, e.g., for administration to a subject to produce an immune response to the exogenous amino acid sequence.

In some embodiments, the virus capsids can be administered to block certain cellular sites prior to and/or concurrently with (e.g., within minutes or hours of each other) administration of a virus vector delivering a nucleic acid encoding a polypeptide or functional RNA of interest. For example, the inventive capsids can be delivered to block cellular receptors on liver cells and a delivery vector can be administered subsequently or concurrently, which may reduce transduction of liver cells, and enhance transduction of other targets (e.g., skeletal, cardiac and/or diaphragm muscle).

According to some embodiments, modified virus capsids can be administered to a subject prior to and/or concurrently with a modified virus vector as described herein. Further, the disclosure provides compositions and pharmaceutical formulations comprising the inventive modified virus capsids; optionally, the composition also comprises a modified virus vector as described herein.

In some embodiments, a nucleic acid (optionally, an isolated nucleic acid) encodes the modified virus capsids and capsid proteins described herein. Further provided are vectors comprising the nucleic acids, and cells (in vivo or in culture) comprising the nucleic acids and/or vectors described herein. As one example, a virus vector may comprise: (a) a modified AAV capsid as described herein; and (b) a nucleic acid comprising at least one terminal repeat sequence, wherein the nucleic acid is encapsidated by the AAV capsid.

Other suitable vectors include without limitation viral vectors (e.g., adenovirus, AAV, herpesvirus, vaccinia, poxviruses, baculovirus, lentivirus, coronavirus, and the like), plasmids, phage, YACs, BACs, and the like. Such nucleic acids, vectors and cells can be used, for example, as reagents (e.g., helper packaging constructs or packaging cells) for the production of modified virus capsids or virus vectors as described herein.

Virus capsids described herein can be produced using any method known in the art, e.g., by using a baculovirus system (Brown et al., (1994) Virology 198:477-488).

The modifications to the AAV capsid protein as described herein are “selective” modifications. This approach is in contrast to previous work with whole subunit or large domain swaps between AAV serotypes (see, e.g., international patent publication WO 00/28004 and Hauck et al., (2003) J. Virology 77:2768-2774). In some embodiments, a “selective” modification results in the insertion and/or substitution and/or deletion of less than or equal to about 20, 18, 15, 12, 10, 9, 8, 7, 6, 5, 4 or 3 contiguous amino acids.

The modified capsid proteins and capsids described herein can further comprise any other modification, now known or later identified. For example, the AAV capsid proteins and virus capsids can be chimeric in that they can comprise all or a portion of a capsid subunit from another virus, optionally another parvovirus or AAV, e.g., as described in international patent publication WO 00/28004.

In some embodiments, the virus capsid can be a targeted virus capsid, comprising a targeting sequence (e.g., substituted or inserted in the viral capsid) that directs the virus capsid to interact with cell-surface molecules present on desired target tissue(s) (see, e.g., International patent publication WO 00/28004 and Hauck et al., (2003) J. Virology 77:2768-2774); Shi et al., Human Gene Therapy 17:353-361 (2006) [describing insertion of the integrin receptor binding motif RGD at positions 520 and/or 584 of the AAV capsid subunit]; and U.S. Pat. No. 7,314,912 [describing insertion of the PI peptide containing an RGD motif following amino acid positions 447, 534, 573 and 587 of the AAV2 capsid subunit]). Other positions within the AAV capsid subunit that tolerate insertions are known in the art (e.g., positions 449 and 588 described by Grifman et al., Molecular Therapy 3:964-975 (2001)).

For example, a virus capsid as described herein may have relatively inefficient tropism toward certain target tissues of interest (e.g., liver, skeletal muscle, heart, diaphragm muscle, kidney, brain, stomach, intestines, skin, endothelial cells, and/or lungs). A targeting sequence can advantageously be incorporated into these low-transduction vectors to thereby confer to the virus capsid a desired tropism and, optionally, selective tropism for particular tissue(s). AAV capsid proteins, capsids and vectors comprising targeting sequences are described, for example in international patent publication WO 00/28004. As another example, one or more non-naturally occurring amino acids as described by Wang et al., Annu Rev Biophys Biomol Struct. 35:225-49 (2006)) can be incorporated into an AAV capsid subunit as described herein at an orthogonal site as a means of redirecting a low-transduction vector to desired target tissue(s). These unnatural amino acids can advantageously be used to chemically link molecules of interest to the AAV capsid protein including without limitation: glycans (mannose—dendritic cell targeting); RGD, bombesin or a neuropeptide for targeted delivery to specific cancer cell types; RNA aptamers or peptides selected from phage display targeted to specific cell surface receptors such as growth factor receptors, integrins, and the like.

In some embodiments, the targeting sequence may be a virus capsid sequence (e.g., an autonomous parvovirus capsid sequence, AAV capsid sequence, or any other viral capsid sequence) that directs infection to a particular cell type(s).

As another nonlimiting example, a heparin or heparan sulfate binding domain (e.g., the respiratory syncytial virus heparin binding domain) may be inserted or substituted into a capsid subunit that does not typically bind HS receptors (e.g., AAV4, AAV5) to confer heparin and/or heparan sulfate binding to the resulting mutant.

B19 infects primary erythroid progenitor cells using globoside as its receptor (Brown et al, (1993) Science 262: 114). The structure of B19 has been determined to 8 Å resolution (Agbandje-McKenna et al, (1994) Virology 203: 106). The region of the B19 capsid that binds to globoside has been mapped between amino acids 399-406 (Chapman et al, (1993) Virology 194:419), a looped out region between β-barrel structures E and F (Chipman et al, (1996) Proc. Nat. Acad. Sci. USA 93:7502). Accordingly, the globoside receptor binding domain of the B19 capsid may be substituted into an AAV capsid protein to target a virus capsid or virus vector comprising the same to erythroid cells.

In some embodiments, the exogenous targeting sequence may be any amino acid sequence encoding a peptide that alters the tropism of a virus capsid or virus vector comprising the modified AAV capsid protein. In some embodiments, the targeting peptide or protein may be naturally occurring or, alternately, completely or partially synthetic. Exemplary targeting sequences include ligands and other peptides that bind to cell surface receptors and glycoproteins, such as ROD peptide sequences, bradykinin, hormones, peptide growth factors (e.g., epidermal growth factor, nerve growth factor, fibroblast growth factor, platelet-derived growth factor, insulin-like growth factors I and II, etc.), cytokines, melanocyte stimulating hormone (e.g., α, β or γ), neuropeptides and endorphins, and the like, and fragments thereof that retain the ability to target cells to their cognate receptors. Other illustrative peptides and proteins include substance P, keratinocyte growth factor, neuropeptide Y, gastrin releasing peptide, interleukin 2, hen egg white lysozyme, erythropoietin, gonadolibcrin, corticostatin, β-endorphin, leu-enkephalin, rimorphin, alpha-neo-enkephalin, angiotensin, pneumadin, vasoactive intestinal peptide, neurotensin, motilin, and fragments thereof as described above. As yet a further alternative, the binding domain from a toxin (e.g., tetanus toxin or snake toxins, such as alpha-bungarotoxin, and the like) can be substituted into the capsid protein as a targeting sequence. In some embodiments, the AAV capsid protein can be modified by substitution of a “nonclassical” import/export signal peptide (e.g., fibroblast growth factor-1 and -2, interleukin 1, HIV-1 Tat protein, herpes virus VP22 protein, and the like) as described by Cleves (Current Biology 7:R318 (1997)) into the AAV capsid protein. Also encompassed are peptide motifs that direct uptake by specific cells, e.g., a FVFLP (SEQ ID NO: 22) peptide motif triggers uptake by liver cells.

Phage display techniques, as well as other techniques known in the art, may be used to identify peptides that recognize any cell type of interest.

The targeting sequence may encode any peptide that targets to a cell surface binding site, including receptors (e.g., protein, carbohydrate, glycoprotein or proteoglycan). Examples of cell surface binding sites include, but are not limited to, heparan sulfate, chondroitin sulfate, and other glycosaminoglycans, sialic acid moieties found on mucins, glycoproteins, and gangliosides, MHC 1 glycoproteins, carbohydrate components found on membrane glycoproteins, including, mannose, N-acetyl-galactosamine, N-acetyl-glucosamine, fucose, galactose, and the like.

In some embodiments, a heparan sulfate (HS) or heparin binding domain is substituted into the virus capsid (for example, in an AAV capsid that otherwise does not bind to HS or heparin). It is known in the art that HS/heparin binding is mediated by a “basic patch” that is rich in arginines and/or lysines. In some embodiments, a sequence following the motif BXXB (SEQ ID NO: 23), where “B” is a basic residue and X is neutral and/or hydrophobic residue can be employed. As a nonlimiting example, BXXB can be RGNR (SEQ ID NO: 24). As another nonlimiting example, BXXB is substituted for amino acid positions 262 through 265 in the native AAV2 capsid protein or at the corresponding position(s) in the capsid protein of another AAV serotype.

Table 7 shows other non-limiting examples of suitable targeting sequences.

TABLE 7
TARGETING SEQUENCES

| | SEQ ID | |
|---|---|---|
| Sequence | NO | Reference |
| NSVRDL(G/S) | 25 | Muller et al., Nature Biotechnology 21: 1040-1046 |
| | | (2003) |
| PRSVTVP | 26 | Muller et al., Nature Biotechnology 21: 1040-1046 |
| | | (2003) |
| NSVSSX(S/A) | 27 | Muller et al., Nature Biotechnology 21: 1040-1046 |
| | | (2003) |
| NGR, NGRAHA | 28 | Grifman et al., Molecular Therapy 3: 964-975 (2001) |
| QPEHSST | 29 | Work et al., Molecular Therapy 13: 683-693 (2006) |
| VNTANST | 30 | Work et al., Molecular Therapy 13: 683-693 (2006) |
| HGPMQS | 31 | Work et al., Molecular Therapy 13: 683-693 (2006) |
| PHKPPLA | 32 | Work et al., Molecular Therapy 13: 683-693 (2006) |
| IKNNEMW | 33 | Work et al., Molecular Therapy 13: 683-693 (2006) |
| RNLDTPM | 34 | Work et al., Molecular Therapy 13: 683-693 (2006) |
| VDSHRQS | 35 | Work et al., Molecular Therapy 13: 683-693 (2006) |
| YDSKTKT | 36 | Work et al., Molecular Therapy 13: 683-693 (2006) |
| SQLPHQK | 37 | Work et al., Molecular Therapy 13: 683-693 (2006) |
| STMQQNT | 38 | Work et al., Molecular Therapy 13: 683-693 (2006) |
| TERYMTQ | 39 | Work et al., Molecular Therapy 13: 683-693 (2006) |
| QPEHSST | 40 | Work et al., Molecular Therapy 13: 683-693 (2006) |
| DASLSTS | 41 | Work et al., Molecular Therapy 13: 683-693 (2006) |
| DLPNKT | 42 | Work et al., Molecular Therapy 13: 683-693 (2006) |
| DLTAARL | 43 | Work et al., Molecular Therapy 13: 683-693 (2006) |
| EPHQFNY | 44 | Work et al., Molecular Therapy 13: 683-693 (2006) |
| EPQSNHT | 45 | Work et al., Molecular Therapy 13: 683-693 (2006) |
| MSSWPSQ | 46 | Work et al., Molecular Therapy 13: 683-693 (2006) |
| NPKHNAT | 47 | Work et al., Molecular Therapy 13: 683-693 (2006) |
| PDGMRTT | 48 | Work et al., Molecular Therapy 13: 683-693 (2006) |
| PNNNKTT | 49 | Work et al., Molecular Therapy 13: 683-693 (2006) |
| QSTTHDS | 50 | Work et al., Molecular Therapy 13: 683-693 (2006) |
| TGSKQKQ | 51 | Work et al., Molecular Therapy 13: 683-693 (2006) |
| SLKHQAL | 52 | Work et al., Molecular Therapy 13: 683-693 (2006) |
| SPIDGEQ | 53 | Work et al., Molecular Therapy 13: 683-693 (2006) |
| WIFPWIQL | 54 | Hajitou et al., TCM 16: 80-88 (2006) |
| CDCRGDCFC | 55 | Hajitou et al., TCM 16: 80-88 (2006) |
| CNGRC | 56 | Hajitou et al., TCM 16: 80-88 (2006) |
| CPRECES | 57 | Hajitou et al., TCM 16: 80-88 (2006) |
| CTTHWGFTLC | 58 | Hajitou et al., TCM 16: 80-88 (2006) |
| CGRRAGGSC | 59 | Hajitou et al., TCM 16: 80-88 (2006) |
| CKGGRAKDC | 60 | Hajitou et al., TCM 16: 80-88 (2006) |
| CVPELGHEC | 61 | Hajitou et al., TCM 16: 80-88 (2006) |
| CRRETAWAK | 62 | Koivunen et al., J. Nucl. Med. 40: 883-888 (1999) |
| VSWFSHRYSPFAVS | 63 | Koivunen et al., J. Nucl. Med. 40: 883-888 (1999) |
| GYRDGYAGPILYN | 64 | Koivunen et al., J. Nucl. Med. 40: 883-888 (1999) |
| XXXY*XXX | 65 | Koivunen et al., J. Nucl. Med. 40: 883-888 (1999) |
| Y*E/MNW | 66 | Koivunen et al., J. Nucl. Med. 40: 883-888 (1999) |
| RPLPPLP | 67 | Koivunen et al., J. Nucl. Med. 40: 883-888 (1999) |
| APPLPPR | 68 | Koivunen et al., J. Nucl. Med. 40: 883-888 (1999) |
| DVFYPYPYASGS | 69 | Koivunen et al., J. Nucl. Med. 40: 883-888 (1999) |
| MYWYPY | 70 | Koivunen et al., J. Nucl. Med. 40: 883-888 (1999) |
| DITWDQLWDLMK | 71 | Koivunen et al., J. Nucl. Med. 40: 883-888 (1999) |
| CWDD(G/L)WLC | 72 | Koivunen et al., J. Nucl. Med. 40: 883-888 (1999) |
| EWCEYLGGYLRCYA | 73 | Koivunen et al., J. Nucl. Med. 40: 883-888 (1999) |
| YXCXXGPXTWXCXP | 74 | Koivunen et al., J. Nucl. Med. 40: 883-888 (1999) |
| IEGPTLRQWLAARA | 75 | Koivunen et al., J. Nucl. Med. 40: 883-888 (1999) |
| LWXX(Y/W/F/H) | 76 | Koivunen et al., J. Nucl. Med. 40: 883-888 (1999) |
| XFXXYLW | 77 | Koivunen et al., J. Nucl. Med. 40: 883-888 (1999) |
| RWGLCD | 78 | Koivunen et al., J. Nucl. Med. 40: 883-888 (1999) |
| MSRPACPPNDKYE | 79 | Koivunen et al., J. Nucl. Med. 40: 883-888 (1999) |
| CLRSGRGC | 80 | Koivunen et al., J. Nucl. Med. 40: 883-888 (1999) |
| CHWMFSPWC | 81 | Koivunen et al., J. Nucl. Med. 40: 883-888 (1999) |
| WXXF | 82 | Koivunen et al., J. Nucl. Med. 40: 883-888 (1999) |
| CSSRLDAC | 83 | Koivunen et al., J. Nucl. Med. 40: 883-888 (1999) |
| CLPVASC | 84 | Koivunen et al., J. Nucl. Med. 40: 883-888 (1999) |
| CGFECVRQCPERC | 85 | Koivunen et al., J. Nucl. Med. 40: 883-888 (1999) |
| CVALCREACGEGC | 86 | Koivunen et al., J. Nucl. Med. 40: 883-888 (1999) |
| SWCEPGWCR | 87 | Koivunen et al., J. Nucl. Med. 40: 883-888 (1999) |
| YSGWGW | 88 | Koivunen et al., J. Nucl. Med. 40: 883-888 (1999) |
| GLSGGRS | 89 | Koivunen et al., J. Nucl. Med. 40: 883-888 (1999) |
| LMLPRAD | 90 | Koivunen et al., J. Nucl. Med. 40: 883-888 (1999) |
| CSCFRDVCC | 91 | Koivunen et al., J. Nucl. Med. 40: 883-888 (1999) |
| CRDVVSVIC | 92 | Koivunen et al., J. Nucl. Med. 40: 883-888 (1999) |
| CNGRC | 93 | Koivunen et al., J. Nucl. Med. 40: 883-888 (1999) |
| MARSGL | 94 | Newton & Deutscher, Phage Peptide Display in |
| | | Handbook of Experimental Pharmacology, pages |
| | | 145-163, Springer-Verlag, Berlin (2008) |
| MARAKE | 95 | Newton & Deutscher, Phage Peptide Display in |
| | | Handbook of Experimental Pharmacology, pages |
| | | 145-163, Springer-Verlag, Berlin (2008) |
| MSRTMS | 96 | Newton & Deutscher, Phage Peptide Display in |
| | | Handbook of Experimental Pharmacology, pages |
| | | 145-163, Springer-Verlag, Berlin (2008) |
| KCCYSL | 97 | Newton & Deutscher, Phage Peptide Display in |
| | | Handbook of Experimental Pharmacology, pages |
| | | 145-163, Springer-Verlag, Berlin (2008) |
| MYWGDSHWLQYWYE | 98 | Newton & Deutscher, Phage Peptide Display in |
| | | Handbook of Experimental Pharmacology, pages |
| | | 145-163, Springer-Verlag, Berlin (2008) |
| MQLPLAT | 99 | Newton & Deutscher, Phage Peptide Display in |
| | | Handbook of Experimental Pharmacology, pages |
| | | 145-163, Springer-Verlag, Berlin (2008) |
| EWLS | 100 | Newton & Deutscher, Phage Peptide Display in |
| | | Handbook of Experimental Pharmacology, pages |
| | | 145-163, Springer-Verlag, Berlin (2008) |
| SNEW | 101 | Newton & Deutscher, Phage Peptide Display in |
| | | Handbook of Experimental Pharmacology, pages |
| | | 145-163, Springer-Verlag, Berlin (2008) |
| TNYL | 102 | Newton & Deutscher, Phage Peptide Display in |
| | | Handbook of Experimental Pharmacology, pages |
| | | 145-163, Springer-Verlag, Berlin (2008) |
| WIFPWIQL | 103 | Newton & Deutscher, Phage Peptide Display in |
| | | Handbook of Experimental Pharmacology, pages |
| | | 145-163, Springer-Verlag, Berlin (2008) |
| WDLAWMFRLPVG | 104 | Newton & Deutscher, Phage Peptide Display in |
| | | Handbook of Experimental Pharmacology, pages |
| | | 145-163, Springer-Verlag, Berlin (2008) |
| CTVALPGGYVRVC | 105 | Newton & Deutscher, Phage Peptide Display in |
| | | Handbook of Experimental Pharmacology, pages |
| | | 145-163, Springer-Verlag, Berlin (2008) |
| CVPELGHEC | 106 | Newton & Deutscher, Phage Peptide Display in |
| | | Handbook of Experimental Pharmacology, pages |
| | | 145-163, Springer-Verlag, Berlin (2008) |
| CGRRAGGSC | 107 | Newton & Deutscher, Phage Peptide Display in |
| | | Handbook of Experimental Pharmacology, pages |
| | | 145-163, Springer-Verlag, Berlin (2008) |
| CVAYCIEHHCWTC | 108 | Newton & Deutscher, Phage Peptide Display in |
| | | Handbook of Experimental Pharmacology, pages |
| | | 145-163, Springer-Verlag, Berlin (2008) |
| CVFAHNYDYLVC | 109 | Newton & Deutscher, Phage Peptide Display in |
| | | Handbook of Experimental Pharmacology, pages |
| | | 145-163, Springer-Verlag, Berlin (2008) |
| CVFTSNYAFC | 110 | Newton & Deutscher, Phage Peptide Display in |
| | | Handbook of Experimental Pharmacology, pages |
| | | 145-163, Springer-Verlag, Berlin (2008) |
| VHSPNKK | 111 | Newton & Deutscher, Phage Peptide Display in |
| | | Handbook of Experimental Pharmacology, pages |
| | | 145-163, Springer-Verlag, Berlin (2008) |
| CDCRGDCFC | 112 | Newton & Deutscher, Phage Peptide Display in |
| | | Handbook of Experimental Pharmacology, pages |
| | | 145-163, Springer-Verlag, Berlin (2008) |
| CRGDGWC | 113 | Newton & Deutscher, Phage Peptide Display in |
| | | Handbook of Experimental Pharmacology, pages |
| | | 145-163, Springer-Verlag, Berlin (2008) |
| XRGCDX | 114 | Newton & Deutscher, Phage Peptide Display in |
| | | Handbook of Experimental Pharmacology, pages |
| | | 145-163, Springer-Verlag, Berlin (2008) |
| PXX(S/T) | 115 | Newton & Deutscher, Phage Peptide Display in |
| | | Handbook of Experimental Pharmacology, pages |
| | | 145-163, Springer-Verlag, Berlin (2008) |
| CTTHWGFTLC | 116 | Newton & Deutscher, Phage Peptide Display in |
| | | Handbook of Experimental Pharmacology, pages |
| | | 145-163, Springer-Verlag, Berlin (2008) |
| SGKGPRQITAL | 117 | Newton & Deutscher, Phage Peptide Display in |
| | | Handbook of Experimental Pharmacology, pages |
| | | 145-163, Springer-Verlag, Berlin (2008) |
| A(A/Q)(N/A)(L/Y) | 118 | Newton & Deutscher, Phage Peptide Display in |
| (T/V/M/R)(R/K) | | Handbook of Experimental Pharmacology, pages |
| | | 145-163, Springer-Verlag, Berlin (2008) |
| VYMSPF | 119 | Newton & Deutscher, Phage Peptide Display in |
| | | Handbook of Experimental Pharmacology, pages |
| | | 145-163, Springer-Verlag, Berlin (2008) |
| MQLPLAT | 120 | Newton & Deutscher, Phage Peptide Display in |
| | | Handbook of Experimental Pharmacology, pages |
| | | 145-163, Springer-Verlag, Berlin (2008) |
| ATWLPPR | 121 | Newton & Deutscher, Phage Peptide Display in |
| | | Handbook of Experimental Pharmacology, pages |
| | | 145-163, Springer-Verlag, Berlin (2008) |
| HTMYYHHYQHHL | 122 | Newton & Deutscher, Phage Peptide Display in |
| | | Handbook of Experimental Pharmacology, pages |
| | | 145-163, Springer-Verlag, Berlin (2008) |
| SEVGCRAGPLQWLCEKYFG | 123 | Newton & Deutscher, Phage Peptide Display in |
| | | Handbook of Experimental Pharmacology, pages |
| | | 145-163, Springer-Verlag, Berlin (2008) |
| CGLLPVGRPDRNVWRWLC | 124 | Newton & Deutscher, Phage Peptide Display in |
| | | Handbook of Experimental Pharmacology, pages |
| | | 145-163, Springer-Verlag, Berlin (2008) |
| CKGQCDRFKGLPWEC | 125 | Newton & Deutscher, Phage Peptide Display in |
| | | Handbook of Experimental Pharmacology, pages |
| | | 145-163, Springer-Verlag, Berlin (2008) |
| SGRSA | 126 | Newton & Deutscher, Phage Peptide Display in |
| | | Handbook of Experimental Pharmacology, pages |
| | | 145-163, Springer-Verlag, Berlin (2008) |
| WGFP | 127 | Newton & Deutscher, Phage Peptide Display in |
| | | Handbook of Experimental Pharmacology, pages |
| | | 145-163, Springer-Verlag, Berlin (2008) |
| LWXXAr | 128 | Newton & Deutscher, Phage Peptide Display in |
| | | Handbook of Experimental Pharmacology, pages |
| | | 145-163, Springer-Verlag, Berlin (2008) |
| XFXXYLW | 129 | Newton & Deutscher, Phage Peptide Display in |
| | | Handbook of Experimental Pharmacology, pages |
| | | 145-163, Springer-Verlag, Berlin (2008) |
| AEPMPHSLNFSQYLWYT | 130 | Newton & Deutscher, Phage Peptide Display in |
| | | Handbook of Experimental Pharmacology, pages |
| | | 145-163, Springer-Verlag, Berlin (2008) |
| WAY(W/F)SP | 131 | Newton & Deutscher, Phage Peptide Display in |
| | | Handbook of Experimental Pharmacology, pages |
| | | 145-163, Springer-Verlag, Berlin (2008) |
| IELLQAR | 132 | Newton & Deutscher, Phage Peptide Display in |
| | | Handbook of Experimental Pharmacology, pages |
| | | 145-163, Springer-Verlag, Berlin (2008) |
| DITWDQLWDLMK | 133 | Newton & Deutscher, Phage Peptide Display in |
| | | Handbook of Experimental Pharmacology, pages |
| | | 145-163, Springer-Verlag, Berlin (2008) |
| AYTKCSRQWRTCMTTH | 134 | Newton & Deutscher, Phage Peptide Display in |
| | | Handbook of Experimental Pharmacology, pages |
| | | 145-163, Springer-Verlag, Berlin (2008) |
| PQNSKIPGPTFLDPH | 135 | Newton & Deutscher, Phage Peptide Display in |
| | | Handbook of Experimental Pharmacology, pages |
| | | 145-163, Springer-Verlag, Berlin (2008) |
| SMEPALPDWWWKMFK | 136 | Newton & Deutscher, Phage Peptide Display in |
| | | Handbook of Experimental Pharmacology, pages |
| | | 145-163, Springer-Verlag, Berlin (2008) |
| ANTPCGPYTHDCPVKR | 137 | Newton & Deutscher, Phage Peptide Display in |
| | | Handbook of Experimental Pharmacology, pages |
| | | 145-163, Springer-Verlag, Berlin (2008) |
| TACHQHVRMVRP | 138 | Newton & Deutscher, Phage Peptide Display in |
| | | Handbook of Experimental Pharmacology, pages |
| | | 145-163, Springer-Verlag, Berlin (2008) |
| VPWMEPAYQRFL | 139 | Newton & Deutscher, Phage Peptide Display in |
| | | Handbook of Experimental Pharmacology, pages |
| | | 145-163, Springer-Verlag, Berlin (2008) |
| DPRATPGS | 140 | Newton & Deutscher, Phage Peptide Display in |
| | | Handbook of Experimental Pharmacology, pages |
| | | 145-163, Springer-Verlag, Berlin (2008) |
| FRPNRAQDYNTN | 141 | Newton & Deutscher, Phage Peptide Display in |
| | | Handbook of Experimental Pharmacology, pages |
| | | 145-163, Springer-Verlag, Berlin (2008) |
| CTKNSYLMC | 142 | Newton & Deutscher, Phage Peptide Display in |
| | | Handbook of Experimental Pharmacology, pages |
| | | 145-163, Springer-Verlag, Berlin (2008) |
| C(R/Q)L/RT(G/N)XX | 143 | Newton & Deutscher, Phage Peptide Display in |
| G(A/V)GC | | Handbook of Experimental Pharmacology, pages |
| | | 145-163, Springer-Verlag, Berlin (2008) |
| CPIEDRPMC | 144 | Newton & Deutscher, Phage Peptide Display in |
| | | Handbook of Experimental Pharmacology, pages |
| | | 145-163, Springer-Verlag, Berlin (2008) |
| HEWSYLAPYPWF | 145 | Newton & Deutscher, Phage Peptide Display in |
| | | Handbook of Experimental Pharmacology, pages |
| | | 145-163, Springer-Verlag, Berlin (2008) |
| MCPKHPLGC | 146 | Newton & Deutscher, Phage Peptide Display in |
| | | Handbook of Experimental Pharmacology, pages |
| | | 145-163, Springer-Verlag, Berlin (2008) |
| RMWPSSTVNLSAGRR | 147 | Newton & Deutscher, Phage Peptide Display in |
| | | Handbook of Experimental Pharmacology, pages |
| | | 145-163, Springer-Verlag, Berlin (2008) |
| SAKTAVSQRVWLPSHRGGEP | 148 | Newton & Deutscher, Phage Peptide Display in |
| | | Handbook of Experimental Pharmacology, pages |
| | | 145-163, Springer-Verlag, Berlin (2008) |
| KSREHVNNSACPSKRITAAL | 149 | Newton & Deutscher, Phage Peptide Display in |
| | | Handbook of Experimental Pharmacology, pages |
| | | 145-163, Springer-Verlag, Berlin (2008) |
| EGFR | 150 | Newton & Deutscher, Phage Peptide Display in |
| | | Handbook of Experimental Pharmacology, pages |
| | | 145-163, Springer-Verlag, Berlin (2008) |
| AGLGVR | 151 | Newton & Deutscher, Phage Peptide Display in |
| | | Handbook of Experimental Pharmacology, pages |
| | | 145-163, Springer-Verlag, Berlin (2008) |
| GTRQGHTMRLGVSDG | 152 | Newton & Deutscher, Phage Peptide Display in |
| | | Handbook of Experimental Pharmacology, pages |
| | | 145-163, Springer-Verlag, Berlin (2008) |
| IAGLATPGWSHWLAL | 153 | Newton & Deutscher, Phage Peptide Display in |
| | | Handbook of Experimental Pharmacology, pages |
| | | 145-163, Springer-Verlag, Berlin (2008) |
| SMSIARL | 154 | Newton & Deutscher, Phage Peptide Display in |
| | | Handbook of Experimental Pharmacology, pages |
| | | 145-163, Springer-Verlag, Berlin (2008) |
| HTFEPGV | 155 | Newton & Deutscher, Phage Peptide Display in |
| | | Handbook of Experimental Pharmacology, pages |
| | | 145-163, Springer-Verlag, Berlin (2008) |
| NTSLKRISNKR1RRK | 156 | Newton & Deutscher, Phage Peptide Display in |
| | | Handbook of Experimental Pharmacology, pages |
| | | 145-163, Springer-Verlag, Berlin (2008) |
| LRIKRKRRKRKKTRK | 157 | Newton & Deutscher, Phage Peptide Display in |
| | | Handbook of Experimental Pharmacology, pages |
| | | 145-163, Springer-Verlag, Berlin (2008) |

Y* is phospho-Tyr

In some embodiments, the targeting sequence may be a peptide that can be used for chemical coupling (e.g., can comprise arginine and/or lysine residues that can be chemically coupled through their R groups) to another molecule that targets entry into a cell.

In some embodiments, the AAV capsid protein or virus capsid can comprise a mutation as described in WO 2006/066066. For example, the capsid protein can comprise a selective amino acid substitution at amino acid position 263, 705, 708 and/or 716 of the native AAV2 capsid protein or a corresponding change(s) in a capsid protein from another AAV serotype.

Additionally, or alternatively, in some embodiments, the capsid protein, virus capsid or vector comprises a selective amino acid insertion directly following amino acid position 264 of the AAV2 capsid protein or a corresponding change in the capsid protein from other AAV. By “directly following amino acid position X” it is intended that the insertion immediately follows the indicated amino acid position (for example, “following amino acid position 264” indicates a point insertion at position 265 or a larger insertion, e.g., from positions 265 to 268, etc.).

Furthermore, in some embodiments, the capsid protein, virus capsid or vector can comprise amino acid modifications such as described in PCT Publication No. WO 2010/093784 (e.g., 2i8) and/or in PCT Publication No. WO 2014/144229 (e.g., dual glycan).

In some embodiments, the capsid protein, virus capsid or vector can have equivalent or enhanced transduction efficiency relative to the transduction efficiency of the AAV serotype from which the capsid protein, virus capsid or vector originated. In some embodiments, the capsid protein, virus capsid or vector can have reduced transduction efficiency relative to the transduction efficiency of the AAV serotype from which the capsid protein, virus capsid or vector originated. In some embodiments, the capsid protein, virus capsid or vector can have equivalent or enhanced tropism relative to the tropism of the AAV serotype from which the capsid protein, virus capsid or vector originated. In some embodiments, the capsid protein, virus capsid or vector can have an altered or different tropism relative to the tropism of the AAV serotype from which the capsid protein, virus capsid or vector originated. In some embodiments, the capsid protein, virus capsid or vector can have or be engineered to have tropism for brain tissue. In some embodiments, the capsid protein, virus capsid or vector can have or be engineered to have tropism for liver tissue.

The AAV vectors described herein can be used to deliver a heterologous nucleic acid to a cell or subject. For example, the modified vector can be used to treat a lysosomal storage disorder such as a mucopolysaccharidosis disorder (e.g., Sly syndrome [3-glucuronidase], Hurler Syndrome [alpha-L-iduronidase], Scheie Syndrome [alpha-L-iduronidase], Hurler-Scheie Syndrome [alpha-L-iduronidase], Hunter's Syndrome [iduronate sulfatase], Sanfilippo Syndrome (A [heparan sulfamidase], B [N-acetylglucosaminidase], C [acetyl-CoA:alpha-glucosaminide acetyltransferase], D [N-acetylglucosamine 6-sulfatase]), Morquio Syndrome (A [galactose-6-sulfate sulfatase], B [3-galactosidase]), Maroteaux-Lamy Syndrome [N-acetylgalactosamine-4-sulfatase], etc.), Fabry disease (a-galactosidase), Gaucher's disease (glucocerebrosidase), or a glycogen storage disorder (e.g., Pompe disease; lysosomal acid alpha-glucosidase) as described herein.

Those skilled in the art will appreciate that for some AAV capsid proteins the corresponding modification will be an insertion and/or a substitution, depending on whether the corresponding amino acid positions are partially or completely present in the virus or, alternatively, are completely absent.

In some embodiments, virus vectors comprise the modified capsid proteins and capsids described herein. In some embodiments, the virus vector is a parvovirus vector (e.g., comprising a parvovirus capsid and/or vector genome), for example, an AAV vector (e.g., comprising an AAV capsid and/or vector genome). In some embodiments, the virus vector comprises a modified AAV capsid comprising a modified capsid as described herein and a vector genome.

For example, in some embodiments, the virus vector comprises: (a) a modified virus capsid (e.g., a modified AAV capsid) comprising a modified capsid protein described herein; and (b) a nucleic acid comprising a terminal repeat sequence (e.g., an AAV TR), wherein the nucleic acid comprising the terminal repeat sequence is encapsidated by the modified virus capsid. The nucleic acid can optionally comprise two terminal repeats (e.g., two AAV TRs).

In some embodiments, the virus vector is a recombinant virus vector comprising a heterologous nucleic acid encoding a polypeptide or functional RNA of interest. Recombinant virus vectors are described in more detail below.

In some embodiments, the virus vectors (i) have reduced transduction of liver as compared with the level of transduction by a virus vector without the modified capsid protein; (ii) exhibit enhanced systemic transduction by the virus vector in an animal subject as compared with the level observed by a virus vector without the modified capsid protein; (iii) demonstrate enhanced movement across endothelial cells as compared with the level of movement by a virus vector without the modified capsid protein, and/or (iv) exhibit a selective enhancement in transduction of muscle tissue (e.g., skeletal muscle, cardiac muscle and/or diaphragm muscle), (v) exhibit a selective enhancement in transduction of liver tissue, and/or (vi) reduced transduction of brain tissues (e.g., neurons) as compared with the level of transduction by a virus vector without the modified capsid protein. In some embodiments, the virus vector has systemic transduction toward liver.

It will be understood by those skilled in the art that the modified capsid proteins, virus capsids and virus vectors described herein exclude those capsid proteins, capsids and virus vectors that have the indicated amino acids at the specified positions in their native state (i.e., are not mutants).

### Methods of Producing Virus Vectors

Also provided herein are methods of producing virus vectors. In some embodiments, a method of producing an AAV vector that evades neutralizing antibodies, comprises: a) identifying contact amino acid residues that form a three dimensional antigenic footprint on an AAV capsid protein; b) generating a library of AAV capsid proteins comprising amino acid substitutions of the contact amino acid residues identified in (a); c) producing AAV particles comprising capsid proteins from the library of AAV capsid proteins of (b); d) contacting the AAV particles of (c) with cells under conditions whereby infection and replication can occur; e) selecting AAV particles that can complete at least one infectious cycle and replicate to titers similar to control AAV particles: 1) contacting the AAV particles selected in (e) with neutralizing antibodies and cells under conditions whereby infection and replication can occur; and g) selecting AAV particles that are not neutralized by the neutralizing antibodies of (f). Nonlimiting examples of methods for identifying contact amino acid residues include peptide epitope mapping and/or cryo-electron microscopy.

Resolution and identification of the antibody contact residues within the three dimensional antigenic footprint allows for their subsequent modification through random, rational and/or degenerate mutagenesis to generate antibody-evading AAV capsids that can be identified through further selection and/or screening.

Thus, in some embodiments, a method of producing an AAV vector that evades neutralizing antibodies comprises: a) identifying contact amino acid residues that form a three dimensional antigenic footprint on an AAV capsid protein; b) generating AAV capsid proteins comprising amino acid substitutions of the contact amino acid residues identified in (a) by random, rational and/or degenerate mutagenesis; c) producing AAV particles comprising capsid proteins from the AAV capsid proteins of (b); d) contacting the AAV particles of (c) with cells under conditions whereby infection and replication can occur; e) selecting AAV particles that can complete at least one infectious cycle and replicate to titers similar to control AAV particles; f) contacting the AAV particles selected in (e) with neutralizing antibodies and cells under conditions whereby infection and replication can occur; and g) selecting AAV particles that are not neutralized by the neutralizing antibodies of (f).

Nonlimiting examples of methods for identifying contact amino acid residues include peptide epitope mapping and/or cryo-electron microscopy. Methods of generating AAV capsid proteins comprising amino acid substitutions of contact amino acid residues by random, rational and/or degenerate mutagenesis are known in the art.

This comprehensive approach presents a platform technology that can be applied to modifying any AAV capsid. Application of this platform technology yields AAV antigenic variants derived from the original AAV capsid template without loss of transduction efficiency. As one advantage and benefit, application of this technology will expand the cohort of patients eligible for gene therapy with AAV vectors.

In some embodiments, a method of producing a virus vector comprises providing to a cell: (a) a nucleic acid template comprising at least one TR sequence (e.g., AAV TR sequence), and (b) AAV sequences sufficient for replication of the nucleic acid template and encapsidation into AAV capsids (e.g., AAV rep sequences and AAV cap sequences encoding the AAV capsids). Optionally, the nucleic acid template further comprises at least one heterologous nucleic acid sequence. In some embodiments, the nucleic acid template comprises two AAV ITR sequences, which are located 5′ and 3′ to the heterologous nucleic acid sequence (if present), although they need not be directly contiguous thereto.

The nucleic acid template and AAV rep and cap sequences are provided under conditions such that virus vector comprising the nucleic acid template packaged within the AAV capsid is produced in the cell. The method can further comprise the step of collecting the virus vector from the cell. The virus vector can be collected from the medium and/or by lysing the cells.

The cell can be a cell that is permissive for AAV viral replication. Any suitable cell known in the art may be employed. In some embodiments, the cell is a mammalian cell. As another option, the cell can be a trans-complementing packaging cell line that provides functions deleted from a replication-defective helper virus, e.g., 293 cells or other E1a trans-complementing cells.

The AAV replication and capsid sequences may be provided by any method known in the art. Current protocols typically express the AAV rep/cap genes on a single plasmid. The AAV replication and packaging sequences need not be provided together, although it may be convenient to do so. The AAV rep and/or cap sequences may be provided by any viral or non-viral vector. For example, the rep/cap sequences may be provided by a hybrid adenovirus or herpesvirus vector (e.g., inserted into the E1a or E3 regions of a deleted adenovirus vector). EBV vectors may also be employed to express the AAV cap and rep genes. One advantage of this method is that EBV vectors are episomal, yet will maintain a high copy number throughout successive cell divisions (i.e., are stably integrated into the cell as extra-chromosomal elements, designated as an “EBV based nuclear episome,” see Margolski, (1992) Curr. Top. Microbiol. Immun. 158:67).

As a further alternative, the rep/cap sequences may be stably incorporated into a cell.

Typically the AAV rep/cap sequences will not be flanked by the TRs, to prevent rescue and/or packaging of these sequences.

The nucleic acid template can be provided to the cell using any method known in the art. For example, the template can be supplied by a non-viral (e.g., plasmid) or viral vector. In some embodiments, the nucleic acid template is supplied by a herpesvirus or adenovirus vector (e.g., inserted into the E1a or E3 regions of a deleted adenovirus). As another illustration, Palombo et al., (1998) J. Virology 72:5025, describes a baculovirus vector carrying a reporter gene flanked by the AAV TRs. EBV vectors may also be employed to deliver the template, as described above with respect to the rep/cap genes.

In some embodiments, the nucleic acid template is provided by a replicating rAAV virus. In some embodiments, an AAV provirus comprising the nucleic acid template is stably integrated into the chromosome of the cell.

To enhance virus titers, helper virus functions (e.g., adenovirus or herpesvirus) that promote a productive AAV infection can be provided to the cell. Helper virus sequences necessary for AAV replication are known in the art. Typically, these sequences will be provided by a helper adenovirus or herpesvirus vector. Alternatively, the adenovirus or herpesvirus sequences can be provided by another non-viral or viral vector, e.g., as a noninfectious adenovirus miniplasmid that carries all of the helper genes that promote efficient AAV production as described by Ferrari et al., (1997) Nature Med. 3: 1295, and U.S. Pat. Nos. 6,040,183 and 6,093,570.

Further, the helper virus functions may be provided by a packaging cell with the helper sequences embedded in the chromosome or maintained as a stable extrachromosomal element. Generally, the helper virus sequences cannot be packaged into AAV virions, e.g., are not flanked by TRs.

Those skilled in the art will appreciate that it may be advantageous to provide the AAV replication and capsid sequences and the helper virus sequences (e.g., adenovirus sequences) on a single helper construct. This helper construct may be a non-viral or viral construct. As one nonlimiting illustration, the helper construct can be a hybrid adenovirus or hybrid herpesvirus comprising the AAV rep/cap genes.

In some embodiments, the AAV rep/cap sequences and the adenovirus helper sequences are supplied by a single adenovirus helper vector. This vector further can further comprise the nucleic acid template. The AAV rep/cap sequences and/or the rAAV template can be inserted into a deleted region (e.g., the Ela or E3 regions) of the adenovirus.

In some embodiments, the AAV rep/cap sequences and the adenovirus helper sequences are supplied by a single adenovirus helper vector. According to this embodiment, the rAAV template can be provided as a plasmid template.

In some embodiments, the AAV rep/cap sequences and adenovirus helper sequences are provided by a single adenovirus helper vector, and the rAAV template is integrated into the cell as a provirus. Alternatively, the rAAV template is provided by an EBV vector that is maintained within the cell as an extrachromosomal element (e.g., as an EBV based nuclear episome).

In some embodiments, the AAV rep/cap sequences and adenovirus helper sequences are provided by a single adenovirus helper. The rAAV template can be provided as a separate replicating viral vector. For example, the rAAV template can be provided by a rAAV particle or a second recombinant adenovirus particle.

According to the foregoing methods, the hybrid adenovirus vector typically comprises the adenovirus 5′ and 3′ cis sequences sufficient for adenovirus replication and packaging (i.e., the adenovirus terminal repeats and PAC sequence). The AAV rep/cap sequences and, if present, the rAAV template are embedded in the adenovirus backbone and are flanked by the 5′ and 3′ cis sequences, so that these sequences may be packaged into adenovirus capsids. As described above, the adenovirus helper sequences and the AAV rep/cap sequences are generally not flanked by TRs so that these sequences are not packaged into the AAV virions. Zhang et al., ((2001) Gene Ther. 18:704-12) describe a chimeric helper comprising both adenovirus and the AAV rep and cap genes.

Herpesvirus may also be used as a helper virus in AAV packaging methods. Hybrid herpesviruses encoding the AAV Rep protein(s) may advantageously facilitate scalable AAV vector production schemes. A hybrid herpes simplex virus type I (HSV-1) vector expressing the AAV-2 rep and cap genes has been described (Conway et al., (1999) Gene Therapy 6:986 and WO 00/17377.

As a further alternative, virus vectors can be produced in insect cells using baculovirus vectors to deliver the rep/cap genes and rAAV template as described, for example, by Urabe et al., (2002) Human Gene Therapy 13: 1935-43.

AAV vector stocks free of contaminating helper virus may be obtained by any method known in the art. For example, AAV and helper virus may be readily differentiated based on size. AAV may also be separated away from helper virus based on affinity for a heparin substrate (Zolotukhin et al. (1999) Gene Therapy 6:973). Deleted replication-defective helper viruses can be used so that any contaminating helper virus is not replication competent. As a further alternative, an adenovirus helper lacking late gene expression may be employed, as only adenovirus early gene expression is required to mediate packaging of AAV virus. Adenovirus mutants defective for late gene expression are known in the art (e.g., ts100K and ts149 adenovirus mutants).

### Recombinant Virus Vectors

The virus vectors described herein are useful for the delivery of nucleic acids to cells in vitro, ex vivo, and in vivo. In particular, the virus vectors can be advantageously employed to deliver or transfer nucleic acids to animal, including mammalian, cells. Thus, in some embodiments, a nucleic acid may be encapsidated by a capsid protein described herein. In some embodiments, the nucleic acid is a cargo nucleic acid. In some embodiments, the cargo nucleic acid comprises a vector genome (e.g., 5′ ITR, transgene, and 3′ ITR).

The cargo nucleic acid sequence delivered by the virus vectors may be any heterologous nucleic acid sequence(s) of interest. Nucleic acids of interest include nucleic acids encoding polypeptides, including therapeutic (e.g., for medical or veterinary uses) or immunogenic (e.g., for vaccines) polypeptides or RNAs. In some embodiments, the cargo nucleic acid comprises a 5′ ITR and a 3′ ITR. In some embodiments, the cargo nucleic acid comprises a 5′ ITR, a transgene, and a 3′ITR. In some embodiments, the transgene encodes a therapeutic protein or RNA.

Therapeutic polypeptides include, but are not limited to, cystic fibrosis transmembrane regulator protein (CFTR), dystrophin (including mini- and micro-dystrophins, see, e.g., Vincent et al, (1993) Nature Genetics 5: 130; U.S. Patent Publication No. 2003/017131; International publication WO/2008/088895, Wang et al., Proc. Natl. Acad. Sci. USA 97: 1 3714-13719 (2000); and Gregorevic et al., Mol. Ther. 16:657-64 (2008)), myostatin propeptide, follistatin, activin type 11 soluble receptor, IGF-1, apolipoproteins such as apoA (apoA1, apoA2, apoA4, apoA-V), apoB (apoB100, ApoB48), apoC (apoCI, apoCII, apoCIII, apoCIV), apoD, apoE, apoH, apoL, apo(a), anti-inflammatory polypeptides such as the Ikappa B dominant mutant, amyloid beta, tau, sarcospan, utrophin (Tinsley et al, (1996) Nature 384:349), mini-utrophin, clotting factors (e.g., Factor VIII, Factor IX, Factor X, etc.), erythropoietin, angiostatin, endostatin, catalase, tyrosine hydroxylase, superoxide dismutase, leptin, the LDL receptor, lipoprotein lipase, progranulin, ornithine transcarbamylase, β-globin, α-globin, spectrin, alpha-1-antitrypsin, adenosine deaminase, hypoxanthine guanine phosphoribosyl transferase, β-glucocerebrosidase, battenin, sphingomyelinase, lysosomal hexosaminidase A, branched-chain keto acid dehydrogenase, frataxin, RP65 protein, cytokines (e.g., alpha-interferon, beta-interferon, gamma-interferon, interleukin-2, interleukin-4, alpha synuclein, parkin, granulocyte-macrophage colony stimulating factor, lymphotoxin, and the like), peptide growth factors, neurotrophic factors and hormones (e.g., somatotropin, insulin, insulin-like growth factors 1 and 2, platelet derived growth factor, epidermal growth factor, fibroblast growth factor, nerve growth factor, neurotrophic factor-3 and -4, brain-derived neurotrophic factor, bone morphogenic proteins [including RANKL and VEGF], glial derived growth factor, transforming growth factor-α and -β, and the like), huntingin, lysosomal acid alpha-glucosidase, iduronate-2-sulfatase, N-sulfoglucosamine sulfohydrolase, alpha-galactosidase A, receptors (e.g., the tumor necrosis growth factor soluble receptor), S100A1, ubiquitin protein ligase E3, parvalbumin, adenylyl cyclase type 6, a molecule that modulates calcium handling (e.g., SERCA2A, Inhibitor 1 of PP1 and fragments thereof [e.g., WO 2006/029319 and WO 2007/100465]), a molecule that effects G-protein coupled receptor kinase type 2 knockdown such as a truncated constitutively active bARKct, anti-inflammatory factors such as IRAP, anti-myostatin proteins, aspartoacylase, monoclonal antibodies (including single chain monoclonal antibodies; an exemplary Mab is the Herceptin® Mab), neuropeptides and fragments thereof (e.g., galanin, Neuropeptide Y (see, U.S. Pat. No. 7,071,172)), angiogenesis inhibitors such as Vasohibins and other VEGF inhibitors (e.g., Vasohibin 2 [see, WO JP2006/073052]). Other illustrative heterologous nucleic acid sequences encode suicide gene products (e.g., thymidine kinase, cytosine deaminase, diphtheria toxin, and tumor necrosis factor), proteins that enhance or inhibit transcription of host factors (e.g., nuclease-dead Cas9 linked to a transcription enhancer or inhibitor element, zinc-finger proteins linked to a transcription enhancer or inhibitor element, transcription activator-like (TAL) effectors linked to a transcription enhancer or inhibitor element), proteins conferring resistance to a drug used in cancer therapy, tumor suppressor gene products (e.g., p53, Rb, Wt-1), TRAIL, FAS-ligand, and any other polypeptide that has a therapeutic effect in a subject in need thereof. AAV vectors can also be used to deliver monoclonal antibodies and antibody fragments, for example, an antibody or antibody fragment directed against myostatin (see, e.g., Fang et al., Nature Biotechnology 23:584-590 (2005)). Heterologous nucleic acid sequences encoding polypeptides include those encoding reporter polypeptides (e.g., an enzyme). Reporter polypeptides are known in the art and include, but are not limited to, Green Fluorescent Protein, β-galactosidase, alkaline phosphatase, luciferase, and chloramphenicol acetyltransferase gene.

Optionally, the heterologous nucleic acid encodes a secreted polypeptide (e.g., a polypeptide that is a secreted polypeptide in its native state or that has been engineered to be secreted, for example, by operable association with a secretory signal sequence as is known in the art).

Alternatively, in some embodiments, the heterologous nucleic acid may encode an antisense nucleic acid, a ribozyme (e.g., as described in U.S. Pat. No. 5,877,022), RNAs that effect spliceosome-mediated/ram-splicing (see, Puttaraju et al, (1999) Nature Biotech. 17:246; U.S. Pat. Nos. 6,013,487; 6,083,702), interfering RNAs (RNAi) including siRNA, shRNA or miRNA that mediate gene silencing (see, Sharp et al, (2000) Science 287:2431), and other non-translated RNAs, such as “guide” RNAs (Gorman et al., (1998) Proc. Nat. Acad. Sci. USA 95:4929; U.S. Pat. No. 5,869,248 to Yuan et al.), and the like. Exemplary untranslated RNAs include RNAi against a multiple drug resistance (MDR) gene product (e.g., to treat and/or prevent tumors and/or for administration to the heart to prevent damage by chemotherapy), RNAi against myostatin (e.g., for Duchenne muscular dystrophy), RNAi against VEGF (e.g., to treat and/or prevent tumors), RNAi against phospholamban (e.g., to treat cardiovascular disease, see, e.g., Andino et al., J. Gene Med. 10: 132-142 (2008) and Li et al., Acta Pharmacol Sin. 26:51-55 (2005)); phospholamban inhibitory or dominant-negative molecules such as phospholamban S16E (e.g., to treat cardiovascular disease, see, e.g., Hoshijima et al. Nat. Med. 8:864-871 (2002)), RNAi to adenosine kinase (e.g., for epilepsy), and RNAi directed against pathogenic organisms and viruses (e.g., hepatitis B and/or C virus, human immunodeficiency virus, CMV, herpes simplex virus, human papilloma virus, etc.).

Further, a nucleic acid sequence that directs alternative splicing can be delivered. To illustrate, an antisense sequence (or other inhibitory sequence) complementary to the 5′ and/or 3′ splice site of dystrophin exon 51 can be delivered in conjunction with a U1 or U7 small nuclear (sn) RNA promoter to induce skipping of this exon. For example, a DNA sequence comprising a U1 or U7 snRNA promoter located 5′ to the antisense/inhibitory sequence(s) can be packaged and delivered in a modified capsid.

In some embodiments, a nucleic acid sequence that directs gene editing can be delivered. For example, the nucleic acid may encode a guide RNA. In some embodiments, the guide RNA is a single guide RNA (sgRNA) comprising a crRNA sequence and a tracrRNA sequence. In some embodiments, the nucleic acid may encode a nuclease. In some embodiments, the nuclease is a zinc-finger nuclease, a homing endonuclease, a TALEN (transcription activator-like effector nuclease), a NgAgo (agronaute endonuclease), a SGN (structure-guided endonuclease), a RGN (RNA-guided nuclease), or modified or truncated variants thereof. In some embodiments, the RNA-guided nuclease is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, or modified or truncated variants thereof. In some embodiments, the Cas9 nuclease is isolated or derived from S. pyogenes or S. aureus.

In some embodiments, a nucleic acid sequence that directs gene knockdown can be delivered. For example, the nucleic acid sequence may encode a siRNA, an shRNA, a microRNA, or an antisense nucleic acid. The virus vector may also comprise a heterologous nucleic acid that shares homology with and recombines with a locus on a host chromosome. This approach can be utilized, for example, to correct a genetic defect in the host cell.

Also provided are virus vectors that express an immunogenic polypeptide, e.g., for vaccination. The nucleic acid may encode any immunogen of interest known in the art including, but not limited to, immunogens from human immunodeficiency virus (HIV), simian immunodeficiency virus (SIV), influenza virus, HIV or SIV gag proteins, tumor antigens, cancer antigens, bacterial antigens, viral antigens, and the like.

The use of parvoviruses as vaccine vectors is known in the art (see, e.g., Miyamura el al, (1994) Proc. Nat. Acad. Sci USA 91:8507; U.S. Pat. No. 5,916,563 to Young et al, U.S. Pat. No. 5,905,040 to Mazzara et al, U.S. Pat. No. 5,882,652, 5,863,541 to Samulski et al). The antigen may be presented in the parvovirus capsid.

Alternatively, the antigen may be expressed from a heterologous nucleic acid introduced into a recombinant vector genome. In some embodiments, any immunogen of interest as described herein and/or as is known in the art can be provided by the virus vectors described herein.

An immunogenic polypeptide can be any polypeptide suitable for eliciting an immune response and/or protecting the subject against an infection and/or disease, including, but not limited to, microbial, bacterial, protozoal, parasitic, fungal and/or viral infections and diseases. For example, the immunogenic polypeptide can be an orthomyxovirus immunogen (e.g., an influenza virus immunogen, such as the influenza virus hemagglutinin (HA) surface protein or the influenza virus nucleoprotein, or an equine influenza virus immunogen) or a lentivirus immunogen (e.g., an equine infectious anemia virus immunogen, a Simian Immunodeficiency Virus (SIV) immunogen, or a Human Immunodeficiency Virus (HIV) immunogen, such as the HIV or SIV envelope GP 160 protein, the HIV or SIV matrix/capsid proteins, and the HIV or SIV gag, pol and env genes products). The immunogenic polypeptide can also be an arenavirus immunogen (e.g., Lassa fever virus immunogen, such as the Lassa fever virus nucleocapsid protein and the Lassa fever envelope glycoprotein), a poxvirus immunogen (e.g., a vaccinia virus immunogen, such as the vaccinia LI or L8 gene products), a flavivirus immunogen (e.g., a yellow fever virus immunogen or a Japanese encephalitis virus immunogen), a filovirus immunogen (e.g., an Ebola virus immunogen, or a Marburg virus immunogen, such as NP and GP gene products), a bunyavirus immunogen (e.g., RVFV, CCHF, and/or SFS virus immunogens), or a coronavirus immunogen (e.g., an infectious human coronavirus immunogen, such as the human coronavirus envelope glycoprotein, or a porcine transmissible gastroenteritis virus immunogen, or an avian infectious bronchitis virus immunogen). The immunogenic polypeptide can further be a polio immunogen, a herpes immunogen (e.g., CMV, EBV, HSV immunogens), a mumps immunogen, a measles immunogen, a rubella immunogen, a diphtheria toxin or other diphtheria immunogen, a pertussis antigen, a hepatitis (e.g., hepatitis A, hepatitis B, hepatitis C, etc.) immunogen, and/or any other vaccine immunogen now known in the art or later identified as an immunogen.

Alternatively, the immunogenic polypeptide can be any tumor or cancer cell antigen. Optionally, the tumor or cancer antigen is expressed on the surface of the cancer cell.

Exemplary cancer and tumor cell antigens are described in S. A. Rosenberg (Immunity 10:281 (1991)). Other illustrative cancer and tumor antigens include, but are not limited to: BRCA1 gene product, BRCA2 gene product, gp100, tyrosinase, GAGE-1/2, BAGE, RAGE, LAGE, NY-ESO-1, CDK-4, β-catenin, MUM-1, Caspase-8, KIAA0205, HPVE, SART-1, FRAME, p15, melanoma tumor antigens (Kawakami et al., (1994) Proc. Natl. Acad. Sci. USA 91:3515; Kawakami et al., (1994) J. Exp. Med., 180:347; Kawakami et al., (1994) Cancer Res. 54:3124), MART-1, gp100, MAGE-1, MAGE-2, MAGE-3, CEA, TRP-1, TRP-2, P-15, tyrosinase (Brichard et al., (1993) J Exp. Med. 178:489); HER-2/neu gene product (U.S. Pat. No. 4,968,603), CA 125, LK26, FB5 (endosialin), TAG 72, AFP, CA 19-9, NSE, DU-PAN-2, CA50, SPan-1, CA72-4, HCG, STN (sialyl Tn antigen), c-erbB-2 proteins, PSA, L-CanAg, estrogen receptor, milk fat globulin, p53 tumor suppressor protein (Levine, (1993) Ann. Rev. Biochem. 62:623); mucin antigens (International Patent Publication No. WO 90/05142); telomerases; nuclear matrix proteins; prostatic acid phosphatase; papilloma virus antigens; and/or antigens now known or later discovered to be associated with the following cancers: melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition or metastasis thereof now known or later identified (see, e.g., Rosenberg, (1996) Ann. Rev. Med. 47:481-91).

As a further alternative, the heterologous nucleic acid can encode any polypeptide that is desirably produced in a cell in vitro, ex vivo, or in vivo. For example, the virus vectors may be introduced into cultured cells and the expressed gene product isolated therefrom.

It will be understood by those skilled in the art that the heterologous nucleic acid(s) of interest can be operably associated with appropriate control sequences. For example, the heterologous nucleic acid can be operably associated with expression control elements, such as transcription/translation control signals, origins of replication, polyadenylation signals, internal ribosome entry sites (IRES), promoters, and/or enhancers, and the like.

Further, regulated expression of the heterologous nucleic acid(s) of interest can be achieved at the post-transcriptional level, e.g., by regulating selective splicing of different introns by the presence or absence of an oligonucleotide, small molecule and/or other compound that selectively blocks splicing activity at specific sites (e.g., as described in WO 2006/119137).

Those skilled in the art will appreciate that a variety of promoter/enhancer elements can be used depending on the level and tissue-specific expression desired. The promoter/enhancer can be constitutive or inducible, depending on the pattern of expression desired. The promoter/enhancer can be native or foreign and can be a natural or a synthetic sequence. By foreign, it is intended that the transcriptional initiation region is not found in the wild-type host into which the transcriptional initiation region is introduced.

In some embodiments, the promoter/enhancer elements can be native to the target cell or subject to be treated. In some embodiments, the promoters/enhancer element can be native to the heterologous nucleic acid sequence. The promoter/enhancer element is generally chosen so that it functions in the target cell(s) of interest. Further, in some embodiments the promoter/enhancer element is a mammalian promoter/enhancer element. The promoter/enhancer element may be constitutive or inducible.

Inducible expression control elements are typically advantageous in those applications in which it is desirable to provide regulation over expression of the heterologous nucleic acid sequence(s). Inducible promoters/enhancer elements for gene delivery can be tissue-specific or -preferred promoter/enhancer elements, and include muscle specific or preferred (including cardiac, skeletal and/or smooth muscle specific or preferred), neural tissue specific or preferred (including brain-specific or preferred), eye specific or preferred (including retina-specific and cornea-specific), liver specific or preferred, bone marrow specific or preferred, pancreatic specific or preferred, spleen specific or preferred, and lung specific or preferred promoter/enhancer elements. Other inducible promoter/enhancer elements include hormone-inducible and metal-inducible elements. Exemplary inducible promoters/enhancer elements include, but are not limited to, a Tet on/off element, a RU486-inducible promoter, an ecdysone-inducible promoter, a rapamycin-inducible promoter, and a metallothionein promoter.

In some embodiments wherein the heterologous nucleic acid sequence(s) is transcribed and then translated in the target cells, specific initiation signals are generally included for efficient translation of inserted protein coding sequences. These exogenous translational control sequences, which may include the ATG initiation codon and adjacent sequences, can be of a variety of origins, both natural and synthetic.

The virus vectors described herein provide a means for delivering heterologous nucleic acids into a broad range of cells, including dividing and non-dividing cells. The virus vectors can be employed to deliver a nucleic acid of interest to a cell in vitro, e.g., to produce a polypeptide in vitro or for ex vivo gene therapy. The virus vectors are additionally useful in a method of delivering a nucleic acid to a subject in need thereof e.g., to express an immunogenic or therapeutic polypeptide or a functional RNA. In this manner, the polypeptide or functional RNA can be produced in vivo in the subject. The subject can be in need of the polypeptide because the subject has a deficiency of the polypeptide. Further, the method can be practiced because the production of the polypeptide or functional RNA in the subject may impart some beneficial effect.

The virus vectors can also be used to produce a polypeptide of interest or functional RNA in cultured cells or in a subject (e.g., using the subject as a bioreactor to produce the polypeptide or to observe the effects of the functional RNA on the subject, for example, in connection with screening methods).

In general, the virus vectors of the described herein can be employed to deliver a heterologous nucleic acid encoding a polypeptide or functional RNA to treat and/or prevent any disease state for which it is beneficial to deliver a therapeutic polypeptide or functional RNA. Illustrative disease states include, but are not limited to: cystic fibrosis (cystic fibrosis transmembrane regulator protein) and other diseases of the lung, hemophilia A (Factor VIII), hemophilia B (Factor IX), thalassemia (β-globin), anemia (erythropoietin) and other blood disorders. Alzheimer's disease (GDF; neprilysin), multiple sclerosis (β-interferon), Parkinson's disease (glial-cell line derived neurotrophic factor [GDNF]), Huntington's disease (RNAi to remove repeats), Canavan's disease, amyotrophic lateral sclerosis, epilepsy (galanin, neurotrophic factors), and other neurological disorders, cancer (endostatin, angiostatin, TRAIL, FAS-ligand, cytokines including interferons; RNAi including RNAi against VEGF or the multiple drug resistance gene product, mir-26a [e.g., for hepatocellular carcinoma]), diabetes mellitus (insulin), muscular dystrophies including Duchenne (dystrophin, mini-dystrophin, insulin-like growth factor I, a sarcoglycan [e.g., α, β, γ], RNAi against myostatic myostatin propeptide, follistatin, activin type II soluble receptor, anti-inflammatory polypeptides such as the Ikappa B dominant mutant, sarcospan, utrophin, mini-utrophin, antisense or RNAi against splice junctions in the dystrophin gene to induce exon skipping [see, e.g., WO/2003/095647], antisense against U7 snRNAs to induce exon skipping [see, e.g., WO/2006/021724], and antibodies or antibody fragments against myostatin or myostatin propeptide) and Becker, Myotonic dystrophy 1 or 2, facioscapulohumeral muscular dystrophy (FSHD), Gaucher disease (glucocerebrosidase), Hurler's disease (a-L-iduronidase), adenosine deaminase deficiency (adenosine deaminase), glycogen storage diseases (e.g., Fabry disease [a-galactosidase] and Pompe disease [lysosomal acid alpha-glucosidase]) and other metabolic disorders, congenital emphysema (alpha-1-antitrypsin), Lesch-Nyhan Syndrome (hypoxan thine guanine phosphoribosyl transferase), Niemann-Pick disease (sphingomyelinase), Tay-Sachs disease (lysosomal hexosaminidase A), frontotemporal dementia, Maple Syrup Urine Disease (branched-chain keto acid dehydrogenase), retinal degenerative diseases (and other diseases of the eye and retina; e.g., PDGF for macular degeneration and/or vasohibin or other inhibitors of VEGF or other angiogenesis inhibitors to treat/prevent retinal disorders, e.g., in Type I diabetes), diseases of solid organs such as brain (including Parkinson's Disease [GDNF], astrocytomas [endostatin, angiostatin and/or RNAi against VEGF], glioblastomas [endostatin, angiostatin and/or RNAi against VEGF]), liver, kidney, heart including congestive heart failure or peripheral artery disease (PAD) (e.g., by delivering protein phosphatase inhibitor 1 (1-1) and fragments thereof (e.g., IIC), serca2a, zinc finger proteins that regulate the phospholamban gene, Barkct, [32-adrenergic receptor, 2-adrenergic receptor kinase (BARK), phosphoinositide-3 kinase (PI3 kinase), S100A1, parvalbumin, adenylyl cyclase type 6, a molecule that effects G-protein coupled receptor kinase type 2 knockdown such as a truncated constitutively active bARKct; calsarcin, RNAi against phospholamban; phospholamban inhibitory or dominant-negative molecules such as phospholamban S16E, etc.), arthritis (insulin-like growth factors), joint disorders (insulin-like growth factor 1 and/or 2), intimal hyperplasia (e.g., by delivering enos, inos), improve survival of heart transplants (superoxide dismutase), AIDS (soluble CD4), muscle wasting (insulin-like growth factor I), kidney deficiency (erythropoietin), anemia (erythropoietin), arthritis (anti-inflammatory factors such as I RAP and TNFa soluble receptor), hepatitis (a-interferon), LDL receptor deficiency (LDL receptor), hyperammonemia (ornithine transcarbamylase), Krabbe's disease (galactocerebrosidase), Batten's disease, spinal cerebral ataxias including SCA1, SCA2 and SCA3, phenylketonuria (phenylalanine hydroxylase), autoimmune diseases, and the like. The compositions and methods disclosed herein can further be used following organ transplantation to increase the success of the transplant and/or to reduce the negative side effects of organ transplantation or adjunct therapies (e.g., by administering immunosuppressant agents or inhibitory nucleic acids to block cytokine production). As another example, bone morphogenic proteins (including BNP 2, 7, etc., RANKL and/or VEGF) can be administered with a bone allograft, for example, following a break or surgical removal in a cancer patient.

In some embodiments, the virus vectors described herein can be employed to deliver a heterologous nucleic acid encoding a polypeptide or functional RNA to treat and/or prevent a liver disease or disorder. The liver disease or disorder may be, for example, primary biliary cirrhosis, nonalcoholic fatty liver disease (NAFLD), non-alcoholic steatohepatitis (NASH), autoimmune hepatitis, hepatitis B, hepatitis C, alcoholic liver disease, fibrosis, jaundice, primary sclerosing cholangitis (PSC), Budd-Chiari syndrome, hemochromatosis, Wilson's disease, alcoholic fibrosis, non-alcoholic fibrosis, liver steatosis, Gilbert's syndrome, biliary atresia, alpha-1-antitrypsin deficiency, alagille syndrome, progressive familial intrahepatic cholestasis, Hemophilia B, Hereditary Angioedema (HAE), Homozygous Familial Hypercholesterolemia (HoFH), Heterozygous Familial Hypercholesterolemia (HeFH), Von Gierke's Disease (GSD 1), Hemophilia A, Methylmalonic Acidemia, Propionic Acidemia, Homocystinuria, Phenylketonuria (PKU), Tyrosinemia Type 1, Arginase 1 Deficiency, Argininosuccinate Lyase Deficiency, Carbamoyl-phosphate synthetase 1 deficiency, Citrullinemia Type 1, Citrin Deficiency, Crigler-Najjar Syndrome Type 1, Cystinosis, Fabry Disease, Glycogen Storage Disease 1b, LPL Deficiency, N-Acetylglutamate Synthetase Deficiency, Ornithine Transcarbamylase Deficiency, Ornithine Translocase Deficiency, Primary Hyperoxaluria Type 1, or ADA SCID.

The compositions and methods described herein can also be used to produce induced pluripotent stem cells (iPS). For example, a virus vector described herein can be used to deliver stem cell associated nucleic acid(s) into a non-pluripotent cell, such as adult fibroblasts, skin cells, liver cells, renal cells, adipose cells, cardiac cells, neural cells, epithelial cells, endothelial cells, and the like.

Nucleic acids encoding factors associated with stem cells are known in the art. Nonlimiting examples of such factors associated with stem cells and pluripotency include Oct-3/4, the SOX family (e.g., SOX 1, SOX2, SOX3 and/or SOX 15), the KIf family (e.g., KIf1, KHZ KIf4 and/or KIf5), the Myc family (e.g., C-myc, L-myc and/or N-myc), NANOG and/or LIN28.

The methods described herein can also be practiced to treat and/or prevent a metabolic disorder such as diabetes (e.g., insulin), hemophilia (e.g., Factor IX or Factor VIII), a lysosomal storage disorder such as a mucopolysaccharidosis disorder (e.g., Sly syndrome [β-glucuronidase], Hurler Syndrome [alpha-L-iduronidase], Scheie Syndrome [alpha-L-iduronidase], Hurler-Scheie Syndrome [alpha-L-iduronidase], Hunter's Syndrome [iduronate sulfatase], Sanfilippo Syndrome A [heparan sulfamidase], B [N-acetylglucosaminidase], C [acetyl-CoA:alpha-glucosaminide acetyltransferase], D [N-acetylglucosamine 6-sulfatase], Morquio Syndrome A [galactoses-sulfate sulfatase], B [3-galactosidase], Maroteaux-Lamy Syndrome [N-acetylgalactosamine-4-sulfatase], etc.), Fabry disease (alpha-galactosidase), Gaucher's disease (glucocerebrosidase), or a glycogen storage disorder (e.g., Pompe disease; lysosomal acid alpha-glucosidase).

Gene transfer has substantial use for understanding and providing therapy for disease states. There are a number of inherited diseases in which defective genes are known and have been cloned. In general, the above disease states fall into two classes: deficiency states, usually of enzymes, which are generally inherited in a recessive manner, and unbalanced states, which may involve regulatory or structural proteins, and which are typically inherited in a dominant manner. For deficiency state diseases, gene transfer can be used to bring a normal gene into affected tissues for replacement therapy, as well as to create animal models for the disease using antisense mutations. For unbalanced disease states, gene transfer can be used to create a disease state in a model system, which can then be used in efforts to counteract the disease state. Thus, virus vectors as described herein permit the treatment and/or prevention of genetic diseases.

The virus vectors described herein may also be employed to provide a functional RNA to a cell in vitro or in vivo. The functional RNA may be, for example, a non-coding RNA. In some embodiments, expression of the functional RNA in the cell can diminish expression of a particular target protein by the cell. Accordingly, functional RNA can be administered to decrease expression of a particular protein in a subject in need thereof. In some embodiments, expression of the functional RNA in the cell can increase expression of a particular target protein by the cell. Accordingly, functional RNA can be administered to increase expression of a particular protein in a subject in need thereof. In some embodiments, expression of the functional RNA can regulate splicing of a particular target RNA in a cell. Accordingly, functional RNA can be administered to regulate splicing a particular RNA in a subject in need thereof. In some embodiments, expression of the functional RNA in the cell can regulate the function of a particular target protein by the cell. Accordingly, functional RNA can be administered to regulate the function of a particular protein in a subject in need thereof. Functional RNA can also be administered to cells in vitro to regulate gene expression and/or cell physiology, e.g., to optimize cell or tissue culture systems or in screening methods.

In addition, virus vectors as described herein find use in diagnostic and screening methods, whereby a nucleic acid of interest is transiently or stably expressed in a cell culture system, or alternatively, a transgenic animal model.

The virus vectors can also be used for various non-therapeutic purposes, including but not limited to use in protocols to assess gene targeting, clearance, transcription, translation, etc., as would be apparent to one skilled in the art. The virus vectors can also be used for the purpose of evaluating safety (spread, toxicity, immunogenicity, etc.). Such data, for example, are considered by the United States Food and Drug Administration as part of the regulatory approval process prior to evaluation of clinical efficacy.

In some embodiments, the virus vectors may be used to produce an immune response in a subject. According to this embodiment, a virus vector comprising a heterologous nucleic acid sequence encoding an immunogenic polypeptide can be administered to a subject, and an active immune response is mounted by the subject against the immunogenic polypeptide. Immunogenic polypeptides are as described hereinabove. In some embodiments, a protective immune response is elicited.

Alternatively, the virus vector may be administered to a cell ex vivo and the altered cell is administered to the subject. The virus vector comprising the heterologous nucleic acid is introduced into the cell, and the cell is administered to the subject, where the heterologous nucleic acid encoding the immunogen can be expressed and induce an immune response in the subject against the immunogen. In some embodiments, the cell is an antigen-presenting cell (e.g., a dendritic cell).

An “active immune response” or “active immunity” is characterized by “participation of host tissues and cells after an encounter with the immunogen. It involves differentiation and proliferation of immunocompetent cells in lymphoreticular tissues, which lead to synthesis of antibody or the development of cell-mediated reactivity, or both.” Herbert B. Herscowitz, Immunophysiology: Cell Function and Cellular Interactions in Antibody Formation, in IMMUNOLOGY: BASIC PROCESSES 117 (Joseph A. Bellanti ed., 1985). Alternatively stated, an active immune response is mounted by the host after exposure to an immunogen by infection or by vaccination. Active immunity can be contrasted with passive immunity, which is acquired through the transfer of preformed substances (antibody, transfer factor, thymic graft, interleukin-2) from an actively immunized host to a non-immune host.

A “protective” immune response or “protective” immunity as used herein indicates that the immune response confers some benefit to the subject in that it prevents or reduces the incidence of disease. Alternatively, a protective immune response or protective immunity may be useful in the treatment and/or prevention of disease, in particular cancer or tumors (e.g., by preventing cancer or tumor formation, by causing regression of a cancer or tumor and/or by preventing metastasis and/or by preventing growth of metastatic nodules). The protective effects may be complete or partial, as long as the benefits of the treatment outweigh any disadvantages thereof.

In some embodiments, the virus vector or cell comprising the heterologous nucleic acid can be administered in an immunogenically effective amount, as described below.

In some embodiments, the virus vectors can be administered for cancer immunotherapy by administration of a virus vector expressing one or more cancer cell antigens (or an immunologically similar molecule) or any other immunogen that produces an immune response against a cancer cell. To illustrate, an immune response can be produced against a cancer cell antigen in a subject by administering a virus vector comprising a heterologous nucleic acid encoding the cancer cell antigen, for example to treat a patient with cancer and/or to prevent cancer from developing in the subject. The virus vector may be administered to a subject in vivo or by using ex vivo methods, as described herein.

Alternatively, the cancer antigen can be expressed as part of the virus capsid or be otherwise associated with the virus capsid (e.g., as described above).

As another alternative, any other therapeutic nucleic acid (e.g., RNAi) or polypeptide (e.g., cytokine) known in the art can be administered to treat and/or prevent cancer.

As used herein, the term “cancer” encompasses tumor-forming cancers. Likewise, the term “cancerous tissue” encompasses tumors. A “cancer cell antigen” encompasses tumor antigens.

The term “cancer” has its understood meaning in the art, for example, an uncontrolled growth of tissue that has the potential to spread to distant sites of the body (i.e., metastasize). Exemplary cancers include, but are not limited to melanoma, adenocarcinoma, thymoma, lymphoma (e.g., non-Hodgkin's lymphoma, Hodgkin's lymphoma), sarcoma, lung cancer, liver cancer, colon cancer, leukemia, uterine cancer, breast cancer, prostate cancer, ovarian cancer, cervical cancer, bladder cancer, kidney cancer, pancreatic cancer, brain cancer and any other cancer or malignant condition now known or later identified. In some embodiments, a method of treating and/or preventing tumor-forming cancers is provided.

The term “tumor” is also understood in the art, for example, as an abnormal mass of undifferentiated cells within a multicellular organism. Tumors can be malignant or benign. In some embodiments, the methods disclosed herein are used to prevent and treat malignant tumors.

By the terms “treating cancer,” “treatment of cancer” and equivalent terms it is intended that the severity of the cancer is reduced or at least partially eliminated and/or the progression of the disease is slowed and/or controlled and/or the disease is stabilized. In some embodiments, these terms indicate that metastasis of the cancer is prevented or reduced or at least partially eliminated and/or that growth of metastatic nodules is prevented or reduced or at least partially eliminated.

By the terms “prevention of cancer” or “preventing cancer” and equivalent terms it is intended that the methods at least partially eliminate or reduce and/or delay the incidence and/or severity of the onset of cancer. Alternatively stated, the onset of cancer in the subject may be reduced in likelihood or probability and/or delayed.

In some embodiments, cells may be removed from a subject with cancer and contacted with a virus vector expressing a cancer cell antigen as described herein. The modified cell is then administered to the subject, whereby an immune response against the cancer cell antigen is elicited. This method can be advantageously employed with immunocompromised subjects that cannot mount a sufficient immune response in vivo (i.e., cannot produce enhancing antibodies in sufficient quantities).

It is known in the art that immune responses may be enhanced by immunomodulatory cytokines (e.g., alpha-interferon, beta-interferon, gamma-interferon, omega-interferon, tau-interferon, interleukin-1-alpha, interleukin-1p, interleukin-2, interleukin-3, interleukin-4, interleukin 5, interleukin-6, interleukin-7, interleukin-8, interleukin-9, interleukin-10, interleukin-11, interleukin-12, interleukin-13, interleukin-14, interleukin-18, B cell Growth factor, CD40 Ligand, tumor necrosis factor-alpha, tumor necrosis factor-β, monocyte chemoattractant protein-1, granulocyte-macrophage colony stimulating factor, and lymphotoxin). Accordingly, immunomodulatory cytokines (preferably, CTL inductive cytokines) may be administered to a subject in conjunction with the virus vector. Cytokines may be administered by any method known in the art. Exogenous cytokines may be administered to the subject, or alternatively, a nucleic acid encoding a cytokine may be delivered to the subject using a suitable vector, and the cytokine produced in vivo.

### Subjects, Pharmaceutical Formulations, and Modes of Administration

Virus vectors and capsids as described herein find use in both veterinary and medical applications. Suitable subjects include both avians and mammals. The term “avian” as used herein includes, but is not limited to, chickens, ducks, geese, quail, turkeys, pheasant, parrots, parakeets, and the like. The term “mammals” as used herein includes, but is not limited to, humans, non-human primates, bovines, ovines, caprines, equines, felines, canines, lagomorphs, etc. Human subjects include neonates, infants, juveniles, adults and geriatric subjects.

In some embodiments, the subject is “in need” of the methods described herein.

In some embodiments, a pharmaceutical composition is provided comprising a virus vector and/or capsid and/or capsid protein and/or virus particle in a pharmaceutically acceptable carrier and, optionally, other medicinal agents, pharmaceutical agents, stabilizing agents, buffers, carriers, adjuvants, diluents, etc. For injection, the carrier will typically be a liquid. For other methods of administration, the carrier may be either solid or liquid. For inhalation administration, the carrier will be respirable, and optionally can be in solid or liquid particulate form.

By “pharmaceutically acceptable” it is meant a material that is not toxic or otherwise undesirable, i.e., the material may be administered to a subject without causing any undesirable biological effects.

Also provided herein are method of transferring a nucleic acid to a cell in vitro. The virus vector may be introduced into the cells at the appropriate multiplicity of infection according to standard transduction methods suitable for the particular target cells. Titers of virus vector to administer can vary, depending upon the target cell type and number, and the particular virus vector, and can be determined by those of skill in the art without undue experimentation. In some embodiments, at least about 10³ infectious units, optionally at least about 10⁵ infectious units are introduced to the cell.

The cell(s) into which the virus vector is introduced can be of any type, including but not limited to neural cells (including cells of the peripheral and central nervous systems, in particular, brain cells such as neurons and oligodendrocytes), lung cells, cells of the eye (including retinal cells, retinal pigment epithelium, and corneal cells), epithelial cells (e.g., gut and respiratory epithelial cells), muscle cells (e.g., skeletal muscle cells, cardiac muscle cells, smooth muscle cells and/or diaphragm muscle cells), dendritic cells, pancreatic cells (including islet cells), hepatic cells, myocardial cells, bone cells (e.g., bone marrow stem cells), hematopoietic stem cells, spleen cells, keratinocytes, fibroblasts, endothelial cells, prostate cells, germ cells, and the like. In some embodiments, the cell can be any progenitor cell. As a further possibility, the cell can be a stem cell (e.g., neural stem cell, liver stem cell). As still a further alternative, the cell can be a cancer or tumor cell. Moreover, the cell can be from any species of origin, as indicated above.

The virus vector can be introduced into cells in vitro for the purpose of administering the modified cell to a subject. In some embodiments, the cells have been removed from a subject, the virus vector is introduced therein, and the cells are then administered back into the subject. Methods of removing cells from the subject for manipulation ex vivo, followed by introduction back into the subject are known in the art (see, e.g., U.S. Pat. No. 5,399,346). Alternatively, the recombinant virus vector can be introduced into cells from a donor subject, into cultured cells, or into cells from any other suitable source, and the cells are administered to a subject in need thereof (i.e., a “recipient” subject).

Suitable cells for ex vivo nucleic acid delivery are as described above. Dosages of the cells to administer to a subject will vary upon the age, condition and species of the subject, the type of cell, the nucleic acid being expressed by the cell, the mode of administration, and the like. Typically, at least about 10² to about 10⁸ cells or at least about 10³ to about 10⁶ cells will be administered per dose in a pharmaceutically acceptable carrier. In some embodiments, the cells transduced with the virus vector are administered to the subject in a therapeutically effective amount in combination with a pharmaceutical carrier.

In some embodiments, the virus vector is introduced into a cell and the cell can be administered to a subject to elicit an immunogenic response against the delivered polypeptide (e.g., expressed as a transgene or in the capsid). Typically, a quantity of cells expressing an immunogenically effective amount of the polypeptide in combination with a pharmaceutically acceptable carrier is administered. An “immunogenically effective amount” is an amount of the expressed polypeptide that is sufficient to evoke an active immune response against the polypeptide in the subject to which the pharmaceutical formulation is administered. In some embodiments, the dosage is sufficient to produce a protective immune response (as defined above). The degree of protection conferred need not be complete or permanent, as long as the benefits of administering the immunogenic polypeptide outweigh any disadvantages thereof.

Thus, in some embodiments, a method of administering a nucleic acid to a cell comprises contacting the cell with the virus vector, virus particle and/or composition as described herein.

Also provided herein is a method of administering the virus vector, virus particle and/or virus capsid as described herein to a subject. In some embodiments, a method of delivering a nucleic acid to a subject comprises administering to the subject a virus particle, virus vector and/or composition as described herein. Administration of the virus vectors, virus particles and/or capsids to a human subject or an animal in need thereof can be by any means known in the art. Optionally, the virus vector, virus particle and/or capsid is delivered in a therapeutically effective dose in a pharmaceutically acceptable carrier. In some embodiments, a therapeutically effective amount of the virus vector, virus particle and/or capsid is delivered.

The virus vectors and/or capsids described herein can further be administered to elicit an immunogenic response (e.g., as a vaccine). Typically, immunogenic compositions comprise an immunogenically effective amount of virus vector and/or capsid in combination with a pharmaceutically acceptable carrier. Optionally, the dosage is sufficient to produce a protective immune response (as defined above). The degree of protection conferred need not be complete or permanent, as long as the benefits of administering the immunogenic polypeptide outweigh any disadvantages thereof. Subjects and immunogens are as described above.

Dosages of the virus vector and/or capsid to be administered to a subject depend upon the mode of administration, the disease or condition to be treated and/or prevented, the individual subject's condition, the particular virus vector or capsid, and the nucleic acid to be delivered, and the like, and can be determined in a routine manner. Exemplary doses for achieving therapeutic effects are titers of at least about 10⁵, about 10⁶, about 10⁷, about 10⁸, about 10⁹, about 10¹⁰, about 10¹¹, about 10¹², about 10¹³, about 10¹⁴, or about 10¹⁵ transducing units, optionally about 10⁸-10¹³ transducing units. In some embodiments, the dose of AAV may be from about 2.0×10¹³ vg/kg body weight of the subject to about 4.0×10¹³ vg/kg body weight of the subject, such as about 2.0×10¹³ vg/kg, about 2.1×10¹³ vg/kg, about 2.2×10¹³ vg/kg, about 2.3×10¹³ vg/kg, about 2.4×10¹³ vg/kg, about 2.5×10¹³ vg/kg, about 2.6×10¹³ vg/kg, about 2.7×10¹³ vg/kg, about 2.8×10¹³ vg/kg, about 2.9×10¹³ vg/kg, about 3.0×10¹³ vg/kg, about 3.1×10¹³ vg/kg, about 3.2×10¹³ vg/kg, about 3.3×10¹³ vg/kg, about 3.4×10¹³ vg/kg, about 3.5×10¹³ vg/kg, about 3.6×10¹³ vg/kg, about 3.7×10¹³ vg/kg, about 3.8×10¹³ vg/kg, about 3.9×10¹³ vg/kg, or about 4.0×10¹³ vg/kg. In some embodiments, the dose of AAV may be from about 2×10¹³ vg to about 4.0×10¹³ vg, such as about 2.0×10¹³ vg, about 2.1×10¹³ vg, about 2.2×10¹³ vg, about 2.3×10¹³ vg, about 2.4×10¹³ vg, about 2.5×10¹³ vg, about 2.6×10¹³ vg, about 2.7×10¹³ vg, about 2.8×10¹³ vg, about 2.9×10¹³ vg, about 3.0×10¹³ vg, about 3.1×10¹³ vg, about 3.2×10¹³ vg, about 3.3×10¹³ vg, about 3.4×10¹³ vg, about 3.5×10¹³ vg, about 3.6×10¹³ vg, about 3.7×10¹³ vg, about 3.8×10¹³ vg, about 3.9×10¹³ vg, or about 4.0×10¹³ vg.

In some embodiments, more than one administration (e.g., two, three, four or more administrations) may be employed to achieve the desired level of gene expression over a period of various intervals, e.g., daily, weekly, monthly, yearly, etc.

Exemplary modes of administration include oral, rectal, transmucosal, intranasal, inhalation (e.g., via an aerosol), buccal (e.g., sublingual), vaginal, intrathecal, intraocular, transdermal, in utero (or in ovo), parenteral (e.g., intravenous, subcutaneous, intradermal, intramuscular [including administration to skeletal, diaphragm and/or cardiac muscle], intradermal, intrapleural, intracerebral, and intraarticular), topical (e.g., to both skin and mucosal surfaces, including airway surfaces, and transdermal administration), intralymphatic, and the like, as well as direct tissue or organ injection (e.g., to liver, skeletal muscle, cardiac muscle, diaphragm muscle or brain). Administration can also be to a tumor (e.g., in or near a tumor or a lymph node). The most suitable route in any given case will depend on the nature and severity of the condition being treated and/or prevented and on the nature of the particular vector that is being used.

Administration to skeletal muscle includes but is not limited to administration to skeletal muscle in the limbs (e.g., upper arm, lower arm, upper leg, and/or lower leg), back, neck, head (e.g., tongue), thorax, abdomen, pelvis/perineum, and/or digits. Suitable skeletal muscles include but are not limited to abductor digiti minimi (in the hand), abductor digiti minimi (in the foot), abductor hallucis, abductor ossis metatarsi quinti, abductor pollicis brevis, abductor pollicis longus, adductor brevis, adductor hallucis, adductor longus, adductor magnus, adductor pollicis, anconeus, anterior scalene, articularis genus, biceps brachii, biceps femoris, brachialis, brachioradialis, buccinator, coracobrachialis, corrugator supercilii, deltoid, depressor anguli oris, depressor labii inferioris, digastric, dorsal interossei (in the hand), dorsal interossei (in the foot), extensor carpi radialis brevis, extensor carpi radialis longus, extensor carpi ulnaris, extensor digiti minimi, extensor digitorum, extensor digitorum brevis, extensor digitorum longus, extensor hallucis brevis, extensor hallucis longus, extensor indicis, extensor pollicis brevis, extensor pollicis longus, flexor carpi radialis, flexor carpi ulnaris, flexor digiti minimi brevis (in the hand), flexor digiti minimi brevis (in the foot), flexor digitorum brevis, flexor digitorum longus, flexor digitorum profundus, flexor digitorum superficial is, flexor hallucis brevis, flexor hallucis longus, flexor pollicis brevis. flexor pollicis longus, frontalis, gastrocnemius, geniohyoid, gluteus maximus, gluteus medius, gluteus minimus, gracilis, iliocostalis cervicis, iliocostalis lumborum, iliocostalis thoracis, illiacus, inferior gemellus, inferior oblique, inferior rectus, infraspinatus, interspinalis, intertransversi, lateral pterygoid, lateral rectus, latissimus dorsi, levator anguli oris, levator labii superioris, levator labii superioris alaeque nasi, levator palpebrae superioris, levator scapulae, long rotators, longissimus capitis, longissimus cervicis, longissimus thoracis, longus capitis, longus colli, lumbricals (in the hand), lumbricals (in the foot), masseter, medial pterygoid, medial rectus, middle scalene, multifidus, mylohyoid, obliquus capitis inferior, obliquus capitis superior, obturator externus, obturator internus, occipitalis, omohyoid, opponens digiti minimi, opponens pollicis, orbicularis oculi, orbicularis oris, palmar interossei, palmaris brevis, palmaris longus, pectineus, pectoralis major, pectoralis minor, peroneus brevis, peroneus longus, peroneus tertius, piriformis, plantar interossei, plantaris, platysma, popliteus, posterior scalene, pronator quadratus, pronator teres, psoas major, quadratus femoris, quadratus plantae, rectus capitis anterior, rectus capitis lateralis, rectus capitis posterior major, rectus capitis posterior minor, rectus femoris, rhomboid major, rhomboid minor, risorius, sartorius, scalenus minimus, semimembranosus, semispinalis capitis, semispinalis cervicis, semispinalis thoracis, semitendinosus, serratus anterior, short rotators, soleus, spinalis capitis, spinalis cervicis, spinalis thoracis, splenius capitis, splenius cervicis, sternocleidomastoid, sternohyoid, sternothyroid, stylohyoid, subclavius, subscapularis, superior gemellus, superior oblique, superior rectus, supinator, supraspinatus, temporalis, tensor fascia lata, teres major, teres minor, thoracis, thyrohyoid, tibialis anterior, tibialis posterior, trapezius, triceps brachii, vastus intermedius, vastus lateralis, vastus medialis, zygomaticus major, and zygomaticus minor, and any other suitable skeletal muscle as known in the art.

The virus vector and/or capsid can be delivered to skeletal muscle by intravenous administration, intra-arterial administration, intraperitoneal administration, limb perfusion, (optionally, isolated limb perfusion of a leg and/or arm; see, e.g. Arruda et al., (2005) Blood 105: 3458-3464), and/or direct intramuscular injection. In some embodiments, the virus vector and/or capsid is administered to a limb (arm and/or leg) of a subject (e.g., a subject with muscular dystrophy such as Duchenne muscular dystrophy (DMD) or limb-girdle muscular dystrophy (LGMD)) by limb perfusion, optionally isolated limb perfusion (e.g., by intravenous or intra-articular administration). In some embodiments, the virus vectors and/or capsids can advantageously be administered without employing “hydrodynamic” techniques. Tissue delivery (e.g., to muscle) of prior art vectors is often enhanced by hydrodynamic techniques (e.g., intravenous/intravenous administration in a large volume), which increase pressure in the vasculature and facilitate the ability of the vector to cross the endothelial cell barrier. In some embodiments, the viral vectors and/or capsids can be administered in the absence of hydrodynamic techniques such as high volume infusions and/or elevated intravascular pressure (e.g., greater than normal systolic pressure, for example, less than or equal to a 5%, 10%, 15%, 20%, 25% increase in intravascular pressure over normal systolic pressure). Such methods may reduce or avoid the side effects associated with hydrodynamic techniques such as edema, nerve damage and/or compartment syndrome. Administration to cardiac muscle includes administration to the left atrium, right atrium, left ventricle, right ventricle and/or septum. The virus vector and/or capsid can be delivered to cardiac muscle by intravenous administration, intra-arterial administration such as intra-aortic administration, direct cardiac injection (e.g., into left atrium, right atrium, left ventricle, right ventricle), and/or coronary artery perfusion.

Administration to diaphragm muscle can be by any suitable method including intravenous administration, intra-arterial administration, and/or intra-peritoneal administration.

Delivery to a target tissue can also be achieved by delivering a depot comprising the virus vector and/or capsid. In some embodiments, a depot comprising the virus vector and/or capsid is implanted into skeletal, cardiac and/or diaphragm muscle tissue or the tissue can be contacted with a film or other matrix comprising the virus vector and/or capsid. Such implantable matrices or substrates are described in U.S. Pat. No. 7,201,898.

In some embodiments, a virus vector and/or virus capsid according is administered to skeletal muscle, diaphragm muscle and/or cardiac muscle (e.g., to treat and/or prevent muscular dystrophy, heart disease [for example, PAD or congestive heart failure]).

In some embodiments, the compositions and methods described herein are used to treat and/or prevent diseases or disorders of skeletal, cardiac and/or diaphragm muscle. The diseases or disorders of the muscle may be, for example, muscular dystrophy, myopathy, motor neuron disease, and cardiomyopathy. The diseases or disorders of the muscle may be, for example, dystrophinopathies, Duchenne muscular dystrophy, Becker muscular dystrophy, myotonic dystrophies (e.g., myotonic dystrophy 1 and 2), facioscapulohumeral muscular dystrophy (FDHD), Eimery-Dreifuss muscular dystrophy, limb-girdle disease, facioscapulohumeral muscular dystrophy, oculopharyngeal muscular dystrophy, distal muscular dystrophy, congenital muscular dystrophy, juvenile macular dystrophy, centronuclear myopathy, central core myopathy, and inclusion body myositis.

In some embodiments, a method of treating and/or preventing muscular dystrophy in a subject in need thereof is provided, the method comprising: administering a treatment or prevention effective amount of a virus vector to a mammalian subject, wherein the virus vector comprises a heterologous nucleic acid encoding dystrophin, a mini-dystrophin, a micro-dystrophin, myostatin propeptide, follistatin, activin type II soluble receptor, IGF-1, anti-inflammatory polypeptides such as the Ikappa B dominant mutant, sarcospan, utrophin, a micro-dystrophin, laminin-a2, alpha-sarcoglycan, beta-sarcoglycan, gamma-sarcoglycan, delta-sarcoglycan, IGF-1, an antibody or antibody fragment against myostatin or myostatin propeptide, and/or RNAi against myostatin. In some embodiments, the virus vector can be administered to skeletal, diaphragm and/or cardiac muscle as described elsewhere herein.

Alternatively, methods described herein can be practiced to deliver a nucleic acid to skeletal, cardiac or diaphragm muscle, which is used as a platform for production of a polypeptide (e.g., an enzyme) or functional RNA (e.g., RNAi, micro RNA, antisense RNA) that normally circulates in the blood or for systemic delivery to other tissues to treat and/or prevent a disorder (e.g., a metabolic disorder, such as diabetes [e.g., insulin], hemophilia [e.g., Factor IX or Factor VIII], a mucopolysaccharide disorder [e.g., Sly syndrome, Hurler Syndrome, Scheie Syndrome, Hurler-Scheie Syndrome, Hunter's Syndrome, Sanfilippo Syndrome A, B, C, D, Morquio Syndrome, Maroteaux-Lamy Syndrome, etc.] or a lysosomal storage disorder such as Gaucher's disease [glucocerebrosidase] or Fabry disease [a-galactosidase A] or a glycogen storage disorder such as Pompe disease [Iysosomal acid alpha glucosidase]). Other suitable proteins for treating and/or preventing metabolic disorders are described herein. The use of muscle as a platform to express a nucleic acid of interest is described in U.S. Patent publication US 2002/0192189.

In some embodiments, a method of treating and/or preventing a metabolic disorder in a subject in need thereof comprises administering a treatment or prevention effective amount of a virus vector to skeletal muscle of a subject, wherein the virus vector comprises a heterologous nucleic acid encoding a polypeptide, wherein the metabolic disorder is a result of a deficiency and/or defect in the polypeptide. Illustrative metabolic disorders and heterologous nucleic acids encoding polypeptides are described herein. Optionally, the polypeptide is secreted (e.g., a polypeptide that is a secreted polypeptide in its native state or that has been engineered to be secreted, for example, by operable association with a secretory signal sequence as is known in the art). Without being limited by any particular theory, according to this embodiment, administration to the skeletal muscle can result in secretion of the polypeptide into the systemic circulation and delivery to target tissue(s). Methods of delivering virus vectors to skeletal muscle is described in more detail herein.

The methods described herein can also be practiced to produce noncoding RNA, such as antisense RNA, RNAi or other functional RNA (e.g., a ribozyme) for systemic delivery.

In some embodiments, a method of treating and/or preventing congenital heart failure or PAD in a subject in need thereof comprises administering a treatment or prevention effective amount of a virus vector to a mammalian subject, wherein the virus vector comprises a heterologous nucleic acid encoding, for example, a sarcoplasmic endoreticulum Ca²⁺-ATPase (SERCA2a), an angiogenic factor, phosphatase inhibitor I (I-1) and fragments thereof (e.g., 11C), RNAi against phospholamban; a phospholamban inhibitory or dominant-negative molecule such as phospholamban S16E, a zinc finger protein that regulates the phospholamban gene, beta-2-adrenergic receptor, beta-2-adrenergic receptor kinase (BARK), PI3 kinase, calsarcan, a β-adrenergic receptor kinase inhibitor (PARKct), inhibitor 1 of protein phosphatase 1 and fragments thereof (e.g., I1 C), S100A1, parvalbumin, adenylyl cyclase type 6, a molecule that effects G-protein coupled receptor kinase type 2 knockdown such as a truncated constitutively active bARKct, Pim-1, PGC-I α, SOD-1, SOD-2, EC-SOD, kallikrein, HIF, thymosin-p4, mir-1, mir-133, mir-206, mir-208 and/or mir-26a.

Injectables can be prepared in conventional forms, either as liquid solutions or suspensions, solid forms suitable for solution or suspension in liquid prior to injection, or as emulsions. Alternatively, one may administer the virus vector and/or virus capsids in a local rather than systemic manner, for example, in a depot or sustained-release formulation. Further, the virus vector and/or virus capsid can be delivered adhered to a surgically implantable matrix (e.g., as described in U.S. Patent Publication No. US-2004-0013645-A1).

The virus vectors and/or virus capsids disclosed herein can be administered to the lungs of a subject by any suitable means, optionally by administering an aerosol suspension of respirable particles comprised of the virus vectors and/or virus capsids, which the subject inhales. The respirable particles can be liquid or solid. Aerosols of liquid particles comprising the virus vectors and/or virus capsids may be produced by any suitable means, such as with a pressure-driven aerosol nebulizer or an ultrasonic nebulizer, as is known to those of skill in the art. See, e.g., U.S. Pat. No. 4,501,729. Aerosols of solid particles comprising the virus vectors and/or capsids may likewise be produced with any solid particulate medicament aerosol generator, by techniques known in the pharmaceutical art.

The virus vectors and virus capsids can be administered to tissues of the CNS (e.g., brain, eye) and may advantageously result in broader distribution of the virus vector or capsid than would be observed in the absence of the compositions and methods described herein.

In some embodiments, the delivery vectors described herein may be administered to treat diseases of the CNS, including genetic disorders, neurodegenerative disorders, psychiatric disorders and tumors. Illustrative diseases of the CNS include, but are not limited to Adrenomyeloneuropathy (AMN), Alzheimer's disease, Angelman Syndrome, Frontotemporal Dementia, Parkinson's disease, Huntington's disease, Fragile X syndrome, Canavan disease, Leigh's disease, Refsum disease, Tourette syndrome, primary lateral sclerosis, amyotrophic lateral sclerosis, progressive muscular atrophy, Pick's disease, muscular dystrophy, multiple sclerosis, myasthenia gravis, Binswanger's disease, trauma due to spinal cord or head injury, Tay Sachs disease (GM2 Gangliosidosis), Lesch-Nyhan disease, MC4R Obesity, Metachromatic Leukodystrophy (MLD), MPS I (Hurler/Scheie), MPS IIIA (Sanfilippo A), Niemann Pick C1, Rett Syndrome, Spinal Muscular Atrophy (SMA), AADC Deficiency, Monogenic Amyotropic Lateral Sclerosis (ALS), Alpha mannosidosis, Aspartylglucosaminuria, Dravet Syndrome, Giant Axonal Neuropathy, Globoid Cell Leukodystrophy (Krabbe), Glut 1 Deficiency, GM1 Gangliosidosis, Infantile Neuronal Ceroid Lipfuscinosis (INCL, Batten), Juvenile Neuronal Ceroid Lipfuscinosis (JNCL, Batten), Late Infantile Neuronal Ceroid Lipfuscinosis (LINCL, Batten), MPS II (Hunter), MPS IIIB (Sanfilippo B), MPS IIIC (Sanfilippo C), MPS IVA (Morquio Syndrome), MPS VI (Maroteaux-Lamy), Peroxisome Biogenesis Disorders (Zellweger Syndrome Spectrum), Sandhoff Disease (GM2 Gangliosidosis), epilepsy, cerebral infarcts, psychiatric disorders including mood disorders (e.g., depression, bipolar affective disorder, persistent affective disorder, secondary mood disorder), schizophrenia, drug dependency (e.g., alcoholism and other substance dependencies), neuroses (e.g., anxiety, obsessional disorder, somatoform disorder, dissociative disorder, grief, post-partum depression), psychosis (e.g., hallucinations and delusions), dementia, paranoia, attention deficit disorder, psychosexual disorders, sleeping disorders, pain disorders, eating or weight disorders (e.g., obesity, cachexia, anorexia nervosa, and bulemia) and cancers and tumors (e.g., pituitary tumors) of the CNS.

Disorders of the CNS include ophthalmic disorders involving the retina, posterior tract, and optic nerve (e.g., retinitis pigmentosa, diabetic retinopathy and other retinal degenerative diseases, uveitis, age-related macular degeneration, glaucoma).

Most, if not all, ophthalmic diseases and disorders are associated with one or more of three types of indications: (1) angiogenesis, (2) inflammation, and (3) degeneration. The viral vectors described herein can be employed to deliver anti-angiogenic factors; anti-inflammatory factors; factors that retard cell degeneration, promote cell sparing, or promote cell growth and combinations of the foregoing.

Diabetic retinopathy, for example, is characterized by angiogenesis. Diabetic retinopathy can be treated by delivering one or more anti-angiogenic factors either intraocularly (e.g., in the vitreous) or periocularly (e.g., in the sub-Tenon's region). One or more neurotrophic factors may also be co-delivered, either intraocularly (e.g., intravitreally) or periocularly.

Uveitis involves inflammation. One or more anti-inflammatory factors can be administered by intraocular (e.g., vitreous or anterior chamber) administration of a delivery vector.

Retinitis pigmentosa, by comparison, is characterized by retinal degeneration. In some embodiments, retinitis pigmentosa can be treated by intraocular (e.g., vitreal administration) of a delivery vector encoding one or more neurotrophic factors.

Age-related macular degeneration involves both angiogenesis and retinal degeneration. This disorder can be treated by administering the inventive delivery vectors encoding one or more neurotrophic factors intraocularly (e.g., vitreous) and/or one or more anti-angiogenic factors intraocularly or periocularly (e.g., in the sub-Tenon's region).

Glaucoma is characterized by increased ocular pressure and loss of retinal ganglion cells. Treatments for glaucoma include administration of one or more neuroprotective agents that protect cells from excitotoxic damage using the inventive delivery vectors. Such agents include N-methyl-D-aspartate (NMDA) antagonists, cytokines, and neurotrophic factors, delivered intraocularly, optionally intravitreally.

In some embodiments, the compositions and methods described herein may be used to treat seizures, e.g., to reduce the onset, incidence or severity of seizures. The efficacy of a therapeutic treatment for seizures can be assessed by behavioral (e.g., shaking, ticks of the eye or mouth) and/or electrographic means (most seizures have signature electrographic abnormalities). Thus, epilepsy, which is marked by multiple seizures over time, may also be treated.

In some embodiments, a method of treating a subject in need thereof comprises administering to the subject an AAV vector comprising a capsid protein, wherein the capsid protein comprises the amino acid sequence of any one of SEQ ID NO: 165-187. In some embodiments, the AAV vector comprises a capsid protein comprising the amino acid sequence of SEQ ID NO: 175, or a sequence at least 95% identical thereto. In some embodiments, the AAV vector comprises a capsid protein comprising the amino acid sequence of SEQ ID NO: 175, or a sequence at least 95% identical thereto. In some embodiments, the subject has Dravet syndrome. In some embodiments, the subject has Rett syndrome. In some embodiments, the subject has Angelman syndrome. In some embodiments, the subject has Niemann-Pick disease. In some embodiments, the subject has Fragile X syndrome. In some embodiments, the subject has Alzheimer's disease. In some embodiments, the subject has Gaucher's disease. In some embodiments, the subject has Huntington's disease. In some embodiments, the subject has Parkinson's disease. In some embodiments, the subject has Friedrich's ataxia. In some embodiments, the AAV vector is administered to the subject by intracerebroventricular (ICV) injection. In some embodiments, the AAV vector is administered to the subject by intrathecal (IT) injection. In some embodiments, the AAV vector is administered to the subject by intravenous (IV) injection.

In some embodiments, a method of treating a subject in need thereof comprises administering to the subject an AAV vector comprising a capsid protein, wherein the capsid protein comprises the amino acid sequence of SEQ ID NO: 175 or 180, wherein the subject has Dravet syndrome, Rett syndrome, Angelman syndrome, Niemann-Pick disease, or Fragile X syndrome, and wherein the AAV vector is administered to the subject by ICV or IT injection.

In some embodiments, a method of treating a subject in need thereof comprises administering to the subject an AAV vector comprising a capsid protein, wherein the capsid protein comprises the amino acid sequence of SEQ ID NO: 175 or 180, wherein the subject has Gaucher's disease, Huntington's disease, Parkinson's disease, or Friedrich's ataxia, and wherein the AAV vector is administered to the subject by ICV or IT injection.

In some embodiments, somatostatin (or an active fragment thereof) is administered to the brain using a delivery vector to treat a pituitary tumor. According to this embodiment, the delivery vector encoding somatostatin (or an active fragment thereof) is administered by microinfusion into the pituitary. Likewise, such treatment can be used to treat acromegaly (abnormal growth hormone secretion from the pituitary). The nucleic acid (e.g., GenBank Accession No. J00306) and amino acid (e.g., GenBank Accession No. P01166; contains processed active peptides somatostatin-28 and somatostatin-14) sequences of somatostatins are known in the art.

In some embodiments, the vector can comprise a secretory signal as described in U.S. Pat. No. 7,071,172.

In some embodiments, the virus vector and/or virus capsid is administered to the CNS (e.g., to the brain or to the eye). The virus vector and/or capsid may be introduced into the spinal cord, brainstem (medulla oblongata, pons), midbrain (hypothalamus, thalamus, epithalamus, pituitary gland, substantia nigra, pineal gland), cerebellum, telencephalon (corpus striatum, cerebrum including the occipital, temporal, parietal and frontal lobes, cortex, basal ganglia, hippocampus and portaamygdala), limbic system, neocortex, corpus striatum, cerebrum, and inferior colliculus. The virus vector and/or capsid may also be administered to different regions of the eye such as the retina, cornea and/or optic nerve.

The virus vector and/or capsid may be delivered into the cerebrospinal fluid (e.g., by lumbar puncture) for more disperse administration of the delivery vector. The virus vector and/or capsid may further be administered intravascularly to the CNS in situations in which the blood-brain barrier has been perturbed (e.g., brain tumor or cerebral infarct).

The virus vector and/or capsid can be administered to the desired region(s) of the CNS by any route known in the art, including but not limited to, intrathecal, intra-ocular, intracerebral, intraventricular, intravenous (e.g., in the presence of a sugar such as mannitol), intranasal, intra-aural, intra-ocular (e.g., intra-vitreous, sub-retinal, anterior chamber) and peri-ocular (e.g., sub-Tenon's region) delivery as well as intramuscular delivery with retrograde delivery to motor neurons. In some embodiments, the virus vector and/or capsid is administered in a liquid formulation by direct injection (e.g., stereotactic injection) to the desired region or compartment in the CNS. In some embodiments, the virus vector and/or capsid may be provided by topical application to the desired region or by intra-nasal administration of an aerosol formulation. Administration to the eye, may be by topical application of liquid droplets. As a further alternative, the virus vector and/or capsid may be administered as a solid, slow-release formulation (see, e.g., U.S. Pat. No. 7,201,898).

In some embodiments, the virus vector can used for retrograde transport to treat and/or prevent diseases and disorders involving motor neurons (e.g., amyotrophic lateral sclerosis (ALS); spinal muscular atrophy (SMA), etc.). For example, the virus vector can be delivered to muscle tissue from which it can migrate into neurons.

### Numbered Embodiments

Notwithstanding the appended claims, the disclosure sets forth the following numbered embodiments:

1. An adeno-associated virus (AAV) vector comprising (i) a recombinant capsid protein and (ii) a cargo nucleic acid encapsidated by the capsid protein, wherein the capsid protein comprises a peptide having the sequence of any one of SEQ ID NO: 12-20.

2. The AAV vector of embodiment 1, wherein the cargo nucleic acid comprises 5′ and 3′ AAV inverted terminal repeats.

3. The AAV vector of embodiment 1 or 2, wherein the cargo nucleic acid comprises a transgene.

4. The AAV vector of embodiment 3, wherein the transgene encodes a therapeutic protein or RNA.

5. The AAV vector of any one of embodiments 1-4, wherein the recombinant capsid protein has at least 90%, at least 95%, at least 96%, at least 97%, at least 98%, or at least 99% sequence identity to the native sequence of the AAV1, AAV2, AAV3, AAV4, AAV5, AAV6, AAV7, AAV8, AAV9, AAV10, AAV11, AAV12, AAVrh.8, AAVrh.10, AAVrh32.33, AAVrh74, bovine AAV or avian AAV capsid.

6. The AAV vector of embodiment 5, wherein the recombinant capsid protein has at least 90% sequence identity to the native sequence of the AAV9 capsid.

7. The AAV vector of any one of embodiments 1-6, wherein the peptide is located at the amino acid positions corresponding to amino acids 451-458 of the native AAV9 capsid, or the equivalent amino acid residues in AAV1, AAV2, AAV3, AAV4, AAV5, AAV6, AAV7, AAV8, AAV10, AAV11, AAV12, AAVrh.8, AAVrh.10, AAVrh32.33, AAVrh74, bovine AAV or avian AAV, and wherein the peptide is selected from any one of SEQ ID NO: 12-18.

8. The AAV vector of any one of embodiments 1-6, wherein the peptide is located at the amino acid positions corresponding to amino acids 587-594 of the native AAV9 capsid, or the equivalent amino acid residues in AAV1, AAV2, AAV3, AAV4, AAV5, AAV6, AAV7, AAV8, AAV10, AAV11, AAV12, AAVrh.8, AAVrh.10, AAVrh32.33, AAVrh74, bovine AAV or avian AAV, and wherein the peptide is selected from SEQ ID NO: 19 or 20.

9. The AAV vector of embodiment 1, wherein the recombinant capsid protein comprises: a) a first peptide having a sequence of any one of SEQ ID NO: 12-18; and b) a second peptide having a sequence of any one of SEQ ID NO: 19-20.

10. The AAV vector of embodiment 9, wherein the first peptide is at amino acid positions 451-458, and the second peptide is at amino acids 587-594, wherein the amino acid numbering is based on the native AAV9 capsid, or the equivalent amino acid residues in AAV1, AAV2, AAV3, AAV4, AAV5, AAV6, AAV7, AAV8, AAV10, AAV11, AAV12, AAVrh.8, AAVrh.10, AAVrh32.33, AAVrh74, bovine AAV or avian AAV.

11. The AAV vector of any one of embodiments 1-10, wherein the peptide inhibits binding of at least one antibody to the capsid protein.

12. The AAV vector of embodiment 11, wherein the peptide inhibits neutralization of infectivity of the AAV vector by the antibody.

13. The AAV vector of any one of embodiments 1-12, wherein the peptide selectively binds to a receptor expressed on the surface of a cell in the central nervous system (CNS).

14. The AAV vector of embodiment 13, wherein the cell is in the premotor cortex, the thalamus, the cerebellar cortex, the dentate nucleus, the spinal cord, or the dorsal root ganglion.

15. The AAV vector of any one of embodiments 1-14, wherein the peptide selectively binds to a receptor expressed on the surface of a cell in the heart.

16. The AAV vector of any one of embodiments 1-15, wherein the capsid protein further comprises a peptide that modifies the HI loop of the capsid.

17. An adeno-associated virus (AAV) vector comprising (i) a mutant AAV9 capsid protein and (ii) a cargo nucleic acid encapsidated by the capsid protein, wherein the capsid protein comprises a peptide having the sequence X¹-X²-X³-X⁴-X⁵-X⁶-X⁷-X⁸ (SEQ ID NO: 158) at amino acids 451-458 of the native AAV9 capsid protein sequence, wherein the peptide does not occur in the native AAV9 capsid protein sequence.

18. The AAV vector of embodiment 17, wherein X¹ is not I, X² is not N, X³ is not G, X⁴ is not S, X⁵ is not G, X⁶ is not Q, X⁷ is not N, and/or X⁸ is not Q.

19. The AAV vector of embodiment 18, wherein X¹ is S, F, Q, G, K, or R.

20. The AAV vector of embodiment 18 or 19, wherein X² is C, G, R, D, T, or Q.

21. The AAV vector of any one of embodiments 18-20, wherein X³ is Q, V, G, Y, R, F, or D.

22. The AAV vector of any one of embodiments 18-21, wherein X⁴ is P, Q, A, or R.

23. The AAV vector of any one of embodiments 18-22, wherein X⁵ is T, N, A, P, or I.

24. The AAV vector of anyone of embodiments 18-23, wherein X⁶ is V, Q, A, or I.

25. The AAV vector of any one of embodiments 18-24, wherein X⁷ is M, P, R, Q, or N.

26. The AAV vector of any one of embodiments 18-25, wherein X⁸ is N, L, F, E, H, or A.

27. The AAV vector of embodiment 17, wherein X¹ is S, X² is C, X³ is Q, X⁴ is P, X⁵ is T, X⁶ is V, X⁷ is M, and X⁸ is N.

28. The AAV vector of embodiment 17, wherein X¹ is F, X² is G, X³ is V, X⁴ is P, X⁵ is N, X⁶ is Q, X⁷ is P, and X⁸ is L.

29. The AAV vector of embodiment 17, wherein X¹ is Q, X² is R, X³ is G, X⁴ is Q, X⁵ is A, X⁶ is A, X⁷ is P, and X⁸ is F.

30. The AAV vector of embodiment 17, wherein X¹ is G, X² is D, X³ is Y, X⁴ is A, X⁵ is P, X⁶ is I, X⁷ is R, and X⁸ is E.

31. The AAV vector of embodiment 17, wherein X¹ is K, X² is T, X³ is R, X⁴ is R, X⁵ is I, X⁶ is V, X⁷ is Q, and X⁸ is H.

32. The AAV vector of embodiment 17, wherein X¹ is F, X² is G, X³ is F, X⁴ is P, X⁵ is N, X⁶ is Q, X⁷ is P, and X⁸ is L.

33. The AAV vector of embodiment 17, wherein X¹ is R, X² is Q, X³ is D, X⁴ is Q, X⁵ is P, X⁶ is I, X⁷ is N, and X⁸ is A.

34. An adeno-associated virus (AAV) vector comprising (i) a mutant AAV9 capsid protein and (ii) a cargo nucleic acid encapsidated by the capsid protein, wherein the capsid protein comprises a peptide having the sequence X¹-X²-X³-X⁴-X⁵-X⁶-X⁷-X⁸ (SEQ ID NO: 158) at amino acids 587-594 of the native AAV9 capsid protein sequence, wherein the peptide does not occur in the native AAV9 capsid protein sequence.

35. The AAV vector of embodiment 34, wherein X¹ is not A, X² is not Q, X³ is not A, X⁴ is not Q, X⁵ is not A, X⁶ is not Q, X⁷ is not T, and/or X⁸ is not G.

36. The AAV vector of embodiment 35, wherein X¹ is S.

37. The AAV vector of embodiment 35 or 36, wherein X² is K or T.

38. The AAV vector of any one of embodiments 35-37, wherein X³ is V.

39. The AAV vector of any one of embodiments 35-38, wherein X⁴ is E or D.

40. The AAV vector of any one of embodiments 35-39, wherein X⁵ is S.

41. The AAV vector of any one of embodiments 35-40, wherein X⁶ is W or I.

42. The AAV vector of any one of embodiments 35-41, wherein X⁷ is T or A.

43. The AAV vector of any one of embodiments 35-42, wherein X⁸ is E or I.

44. The AAV vector of embodiment 34, wherein X¹ is S, X² is K, X³ is V, X⁴ is E, X⁵ is S, X⁶ is W, X⁷ is T, and X⁸ is E.

45. The AAV vector of embodiment 34, wherein X¹ is S, X² is T, X³ is V, X⁴ is D, X⁵ is S, X⁶ is I, X⁷ is A, and X⁸ is I.

46. An adeno-associated virus (AAV) vector comprising (i) a recombinant capsid protein and (ii) a cargo nucleic acid encapsidated by the capsid protein, wherein the capsid protein comprises an amino acid sequence that is at least 95%, at least 96%, at least 97%, at least 98%, or at least 99% identical to any one of SEQ ID NO: 165-187.

47. The AAV vector of embodiment 46, wherein the capsid protein comprises the amino acid sequence of any one of SEQ ID NO: 165-187.

48. The AAV vector of embodiment 47, wherein the capsid protein comprises the amino acid sequence of SEQ ID NO: 175.

49. The AAV vector of embodiment 47, wherein the capsid protein comprises the amino acid sequence of SEQ ID NO: 180.

50. The AAV vector of any one of embodiments 46-49, wherein the AAV vector selectively delivers the cargo nucleic acid to a cell or tissue of the central nervous system.

51. The AAV vector of embodiment 50, wherein the tissue of the central nervous system is the premotor cortex, the thalamus, the cerebellar cortex, the dentate nucleus, the spinal cord, or the dorsal root ganglion.

52. The AAV vector of any one of embodiments 46-49, wherein the AAV vector delivers the cargo nucleic acid to the brain, but does not deliver the AAV vector to the heart.

53. The AAV vector of any one of embodiments 46-49, wherein the AAV vector delivers the cargo nucleic acid to the brain and to the heart.

54. The AAV vector of embodiment 53, wherein delivery of the cargo nucleic acid is greater to the brain than to the heart.

55. The AAV vector of embodiment 53, wherein delivery of the cargo nucleic acid is approximately equal in the brain in the heart.

56. A nucleic acid sequence encoding the recombinant capsid protein of the AAV vector of any one of embodiments 1 to 55.

57. The nucleic acid sequence of embodiment 56, wherein the nucleic acid sequence is a DNA sequence.

58. The nucleic acid sequence of embodiment 56, wherein the nucleic acid sequence is an RNA sequence.

59. An expression vector comprising the nucleic acid sequence of any one of embodiments 56-58.

60. A cell comprising the nucleic acid sequence of any one of embodiments 56-58.

61. A cell comprising the expression vector of embodiment 59.

62. A pharmaceutical composition comprising the AAV vector of any one of embodiments 1-55.

63. The pharmaceutical composition of embodiment 62, wherein the composition further comprises a pharmaceutically acceptable carrier.

64. A pharmaceutical composition comprising the cell of embodiment 60 or 61.

65. The pharmaceutical composition of embodiment 64, wherein the composition further comprises a pharmaceutically acceptable carrier.

66. A method of treating a subject in need thereof comprising administering to the subject a therapeutically effective amount of the AAV vector of any one of embodiments 1-55.

67. The method of embodiment 66, wherein the subject has a disease or disorder of the central nervous system.

68. The method of embodiment 67, wherein the disease or disorder of the central nervous system is Dravet syndrome.

69. The method of embodiment 67, wherein the disease or disorder of the central nervous system is Rett syndrome.

70. The method of embodiment 67, wherein the disease or disorder of the central nervous system is Angelman syndrome.

71. The method of embodiment 67, wherein the disease or disorder of the central nervous system is Niemann-Pick disease.

72. The method of embodiment 67, wherein the disease or disorder of the central nervous system is Fragile X syndrome.

73. The method of embodiment 67, wherein the disease or disorder of the central nervous system is Gaucher's disease.

74. The method of embodiment 67, wherein the disease or disorder of the central nervous system is Huntington's disease.

75. The method of embodiment 67, wherein the disease or disorder of the central nervous system is Parkinson's disease.

76. The method of embodiment 67, wherein the disease or disorder of the central nervous system is Friedrich's ataxia.

77. The method of any one of embodiments 66-76, wherein AAV vector comprises a capsid protein, wherein the capsid protein comprises the amino acid sequence of any one of SEQ ID NO: 165-187.

78. The method of any one of embodiments 66-76, wherein AAV vector comprises a capsid protein, wherein the capsid protein comprises the amino acid sequence of SEQ ID NO: 175.

79. The method of any one of embodiments 66-76, wherein AAV vector comprises a capsid protein, wherein the capsid protein comprises the amino acid sequence of SEQ ID NO: 180.

80. The method of any one of embodiments 66-79, wherein the AAV vector is administered to the subject by intracerebroventricular (ICV) injection.

81. The method of any one of embodiments 66-79, wherein the AAV vector is administered to the subject by intrathecal (IT) injection.

82. The method of any one of embodiments 66-79, wherein the AAV vector is administered to the subject by intravenous (IV) injection.

83. The method of any one of embodiments 66-82, wherein the subject is a mammal.

84. The method of embodiment 83, wherein the subject is a human.

85. An in vitro method of introducing a nucleic acid molecule into a cell, comprising contacting the cell with the AAV vector of any one of embodiments 1-55.

86. An AAV vector of any one of embodiments 1-55 for use as a medicament.

87. An AAV vector of any one of embodiments 1-55 for use in a method of treatment of a subject in need thereof.

88. An AAV vector of any one of embodiments 1-55 for use in a method of treating or preventing a disease or disorder of the CNS in a subject in need thereof.

### EXAMPLES

The following examples, which are included herein for illustration purposes only, are not intended to be limiting. As used herein, the terms STRD.101 and STRD.102 are used to describe capsid protein sequences, and AAV-STRD.101 and AAV-STRD.102 are used to describe AAV vectors comprising the capsid proteins. However, the terms STRD.101 and STRD.102 may be used in some contexts to describe AAV vectors comprising the named capsids, as will be apparent to the skilled artisan.

### Example 1. Combinatorial Engineering and Selection of Antibody-Evading AAV Vectors

The method for generating antibody evading AAV mutants is as follows. The first step involves identification of conformational 3D antigenic epitopes on the AAV capsid surface, for example using cryo-electron microscopy. Selected residues within antigenic motifs are then subjected to mutagenesis using degenerate primers with each codon substituted by nucleotides NNK and gene fragments combined together by Gibson assembly and/or multistep PCR. Capsid-encoding genes containing a degenerate library of mutated antigenic motifs are cloned into a wild type AAV genome to replace the original Cap encoding DNA sequence, yielding a plasmid library. Plasmid libraries are then transfected into 293 producer cell lines with an adenoviral helper plasmid to generate AAV capsid libraries, which can then be subjected to selection. Successful generation of AAV libraries is confirmed via DNA sequencing.

In order to select for new AAV strains that can escape neutralizing antibodies (NAbs) and/or target the central nervous system (CNS), AAV libraries are subjected to multiple rounds of infection in non-human primates. At each stage, tissues of interest are isolated from animal subjects. Cell lysates harvested from the tissues of interest are sequenced to identify AAV isolates escaping antibody neutralization. After multiple rounds of infection in non-human primates, the isolated sequences from each mutagenized region are combined in all permutations and combinations.

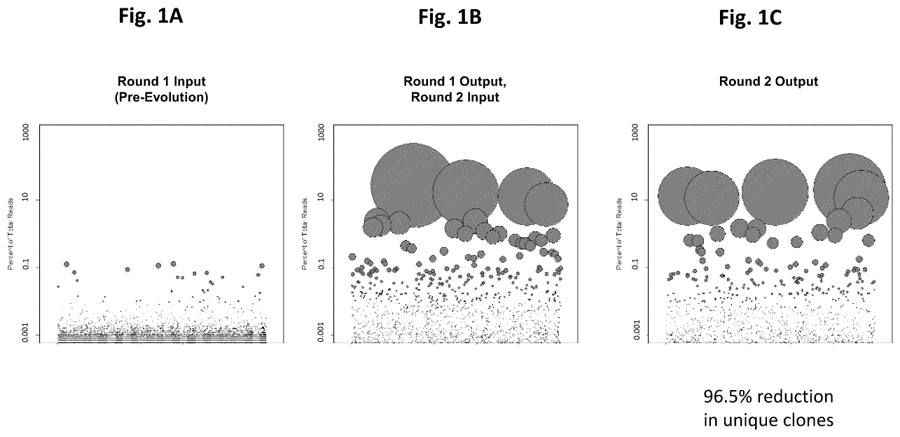

As a specific example, a common antigenic motif on an AAV capsid protein (VP1) was subjected to mutagenesis as described above. The degenerate libraries (FIG. 1A) were then subjected to a first round of infection in a non-human primate (intravenous injection). Tissues were harvested at day 7 post-infection and sequenced to identify single AAV isolates.

Various recombinant AAV isolates were identified in tissue samples, including the spinal cord, dorsal root ganglion, frontal lobe, temporal lobe, occipital lobe, putamen, globus pallidus, thalamus, amygdala, hippocampus, substantia nigra, pons, cerebellum, medulla. Results from this first round of evolution are shown in FIG. 1B.

The recombinant AAVs isolated during the first round of evolution (FIG. 1B) were then reintroduced into a second non-human primate. Tissues were harvested at day 7 post-infection and sequenced to identify single AAV isolates. The results from this second round of evolution are shown in FIG. 1C.

Recombinant AAVs with the highest frequency were sequenced. Substitutions present in these AAVs are shown in Tables 6.1 and 6.2. These data demonstrate that recombinant AAV virions having capsid proteins comprising the substitutions listed in Tables 6.1 and 6.2 evade neutralizing antibodies in vivo in non-human primates and have a tropism for the desired target tissues.

### Example 2: Manufacturability of Recombinant AAV Vectors

To determine whether various recombinant AAVs identified in Example 1 may be manufactured in large-scale systems, the AAVs were produced according to standard methods, and yield was compared to that of wildtype AAV vectors.

AAVs were produced in HEK293 cells according to a standard triple transfection protocol. Briefly, the cells were transfected with (i) a plasmid comprising either the wildtype AAV9 capsid sequence, the STRD.101 capsid variant sequence (SEQ ID NO: 180), or the STRD.102 capsid variant sequence (SEQ ID NO: 175), (ii) a plasmid comprising a 5′ITR, a transgene, and a 3′ ITR sequence, and (ii) a plasmid comprising helper genes necessary for AAV production. Two different transgenes were used with each capsid, in self-complementary constructs. The cells were subsequently lysed and the virions were purified using an affinity column, CsCl density ultracentrifugation, and dialysis. Subsequently, yield of each AAV was measured using a PCR-based quantification approach.

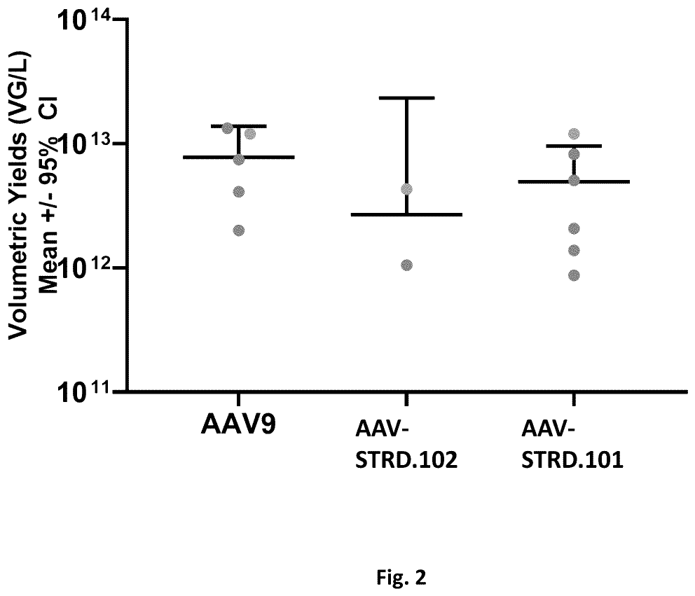

As shown in FIG. 2, recombinant AAV vectors comprising the STRD.101 and STRD.102 capsids had a yield that was similar to the yield of wildtype AAV9. This data confirms that recombinant AAVs comprising the recombinant capsid proteins are suitable for commercial manufacturing.

### Example 3: In Vitro Transduction Using Recombinant AAV Viral Vectors

To confirm whether the recombinant AAV vectors of Example 1 are generally infective and able to transduce cells in culture, various AAV vectors were prepared according to a standard protocol.

The infectivity of the recombinant AAVs was tested using a standard TCID50 assay. Briefly, HeLaRC32 cells were infected with recombinant AAV particles in the presence of Adenovirus (Ad5) at doses spanning 5 orders of magnitude. After 72 hours, DNA was extracted and vector genome replication was quantified by qPCR.

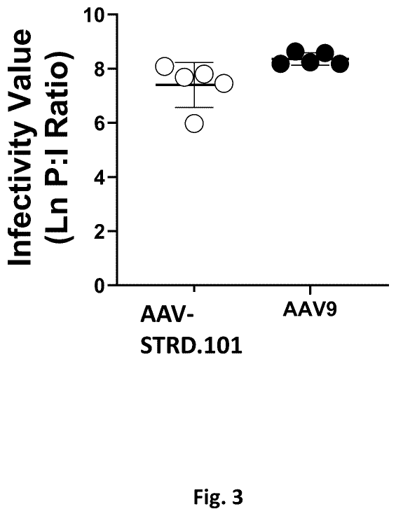

The particle to infectivity ratio was calculated to determine infectivity. As shown in FIG. 3, the infectivity ratio of an AAV-STRD.101 vector was lower compared to that of wildtype AAV9. Because a lower infectivity ratio translates to a higher potency, AAV-STRD.101 is more infectious than wildtype AAV9.

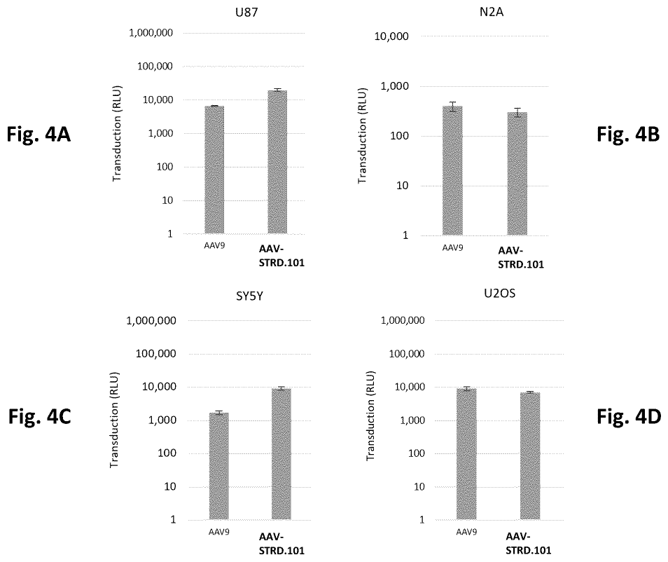

Separately, infectivity was also determined in various cell lines. Recombinant AAVs packaging a luciferase transgene were generated and contacted with the cells in culture at a dose of 10,000 vector genomes (vg) per cell. 48-hours post infection, cells were lysed. The lysate was contacted with a bioluminescent substrate, and relative fluorescence units (RFUs) were measured. As shown in FIG. 4A-4D, AAV-STRD.101 vectors infected U87 cells (human glioblastoma cell line, FIG. 4A), N2A cells (mouse neural crest-derived cell line, FIG. 4B), SY5Y cells (human neuroblastoma cell line, FIG. 4C), and U2OS cells (human osteosarcoma cell line, FIG. 4D) at levels comparable to wildtype AAV9.

Accordingly, this data demonstrates that the recombinant AAV vectors of Example 1 can effectively transduce cells in culture.

### Example 4: In Vivo Characterization of Recombinant AAVs Targeting the Central Nervous System

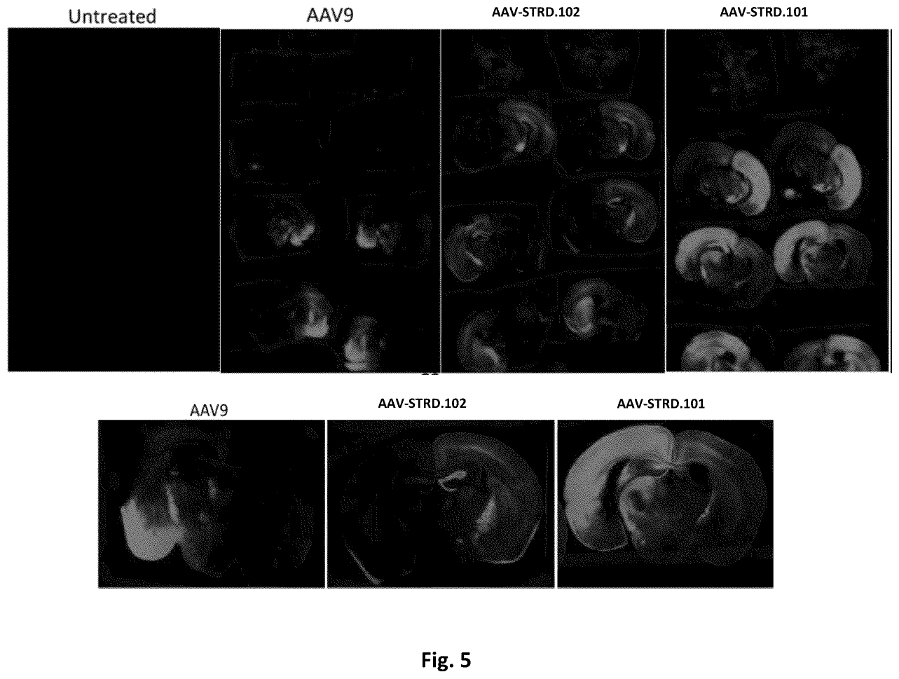

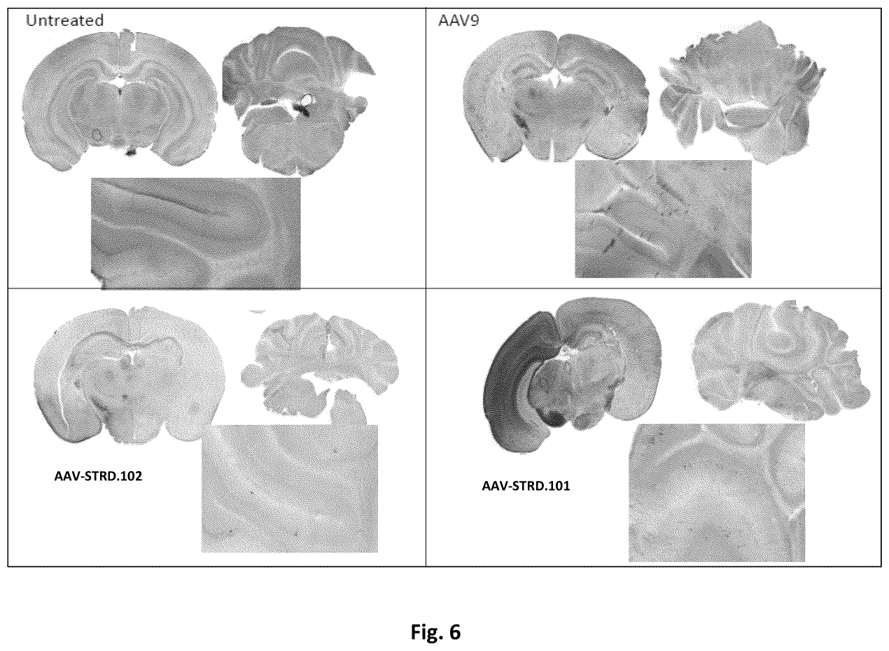

Two recombinant capsid proteins, STRD.101 and STRD.102 were selected for in vivo characterization. Recombinant AAVs comprising these capsid proteins and packaging a native tdTomato fluorescent transgene were generated. The recombinant AAVs were administered to neonatal mice by intracerebroventricular injection at day 0. At three weeks post-injection, brain tissues were harvested and fixed to evaluate the expression by visual assessment of the tdTomato fluorescence. FIG. 5 provides representative images showing tdTomato expression in coronal vibratome sections after 24 hours post-fixation with 4% PFA. These same sections were also visualized using immunohistochemistry (FIG. 6). As shown in the images of FIG. 5 and FIG. 6, AAV9, AAV-STRD.102 and AAV-STRD.101 vectors each had different distribution in the brain tissues, with the highest transgene expression localized near the site of injection. Taken together, this data shows that the recombinant AAVs tested successfully deliver a transgene to target cells in vivo after intracerbroventricular injection.

The AAV-STRD.101 and AAV-STRD.102 vectors packaging tdTomato were also administered to four adult mice by intravenous injection at a dose of 5.5×10¹³ vg/kg. Three weeks post-injection, liver and heart were harvested and fixed to evaluate the expression profile by visual assessment of tdTomato fluorescence.

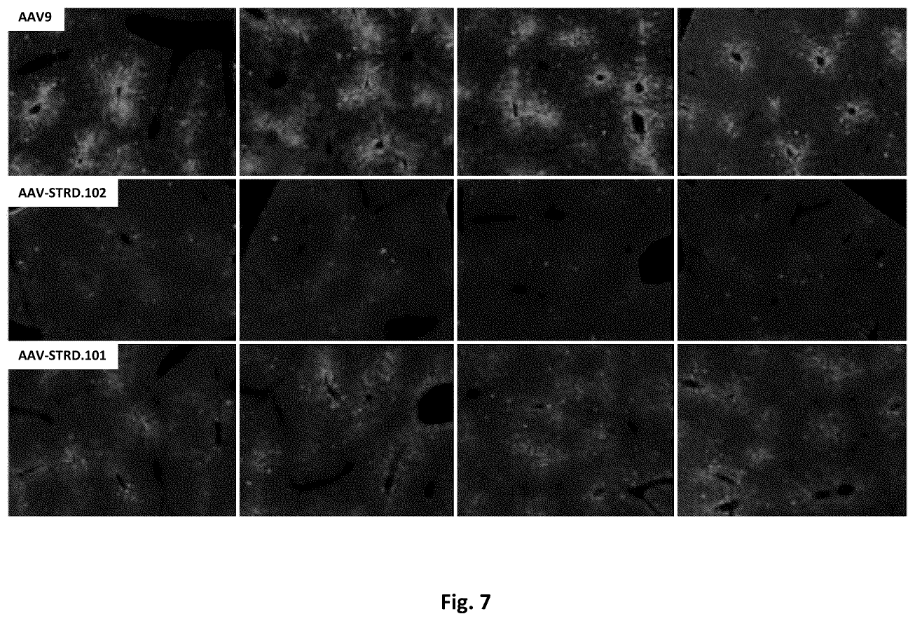

Representative images from one mouse showing TdTomato expression in vibratrome liver sections after 24 hours post-fixation with 4% PFA are provided in FIG. 7. Notably, the AAV-STRD.102 and AAV-STRD.101 vectors were detargeted to the liver compared to wildtype AAV9. This desirable property was unexpected, as no counter screen in the liver was performed during evolution.

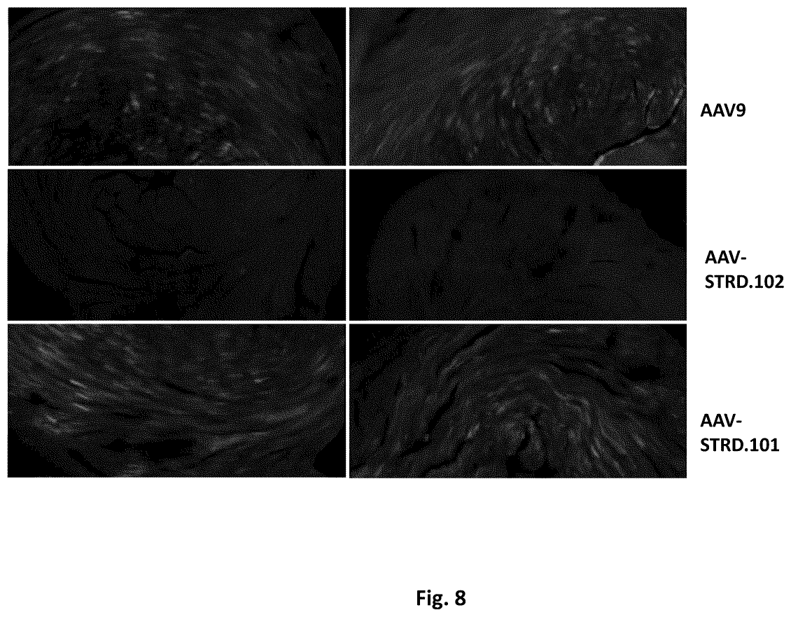

Representative images from one mouse showing TdTomato expression in vibratrome heart sections after 24 hours post-fixation with 4% PFA are provided in FIG. 8. Notably, the vectors tested had different tropism for the heart. Specifically, the AAV-STRD.102 vector was less infective in heart compared to AAV-STRD.101. Because no heart screen was performed during evolution, this differential transduction was wholly unexpected.

Taken together, this data indicates that the AAV-STRD.102 and AAV-STRD.101 vectors can be successfully used to target CNS tissues in vivo, avoid clearance by the liver, and are powerful tools for gene therapy. Given their different tropisms (i.e., AAV-STRD.101 was more infective in the heart than AAV-STRD.102), these vectors will be powerful tools for targeting gene therapy treatments to specifically desired tissues.

### Example 5: Biodistribution of Recombinant AAVs in Non-Human Primates

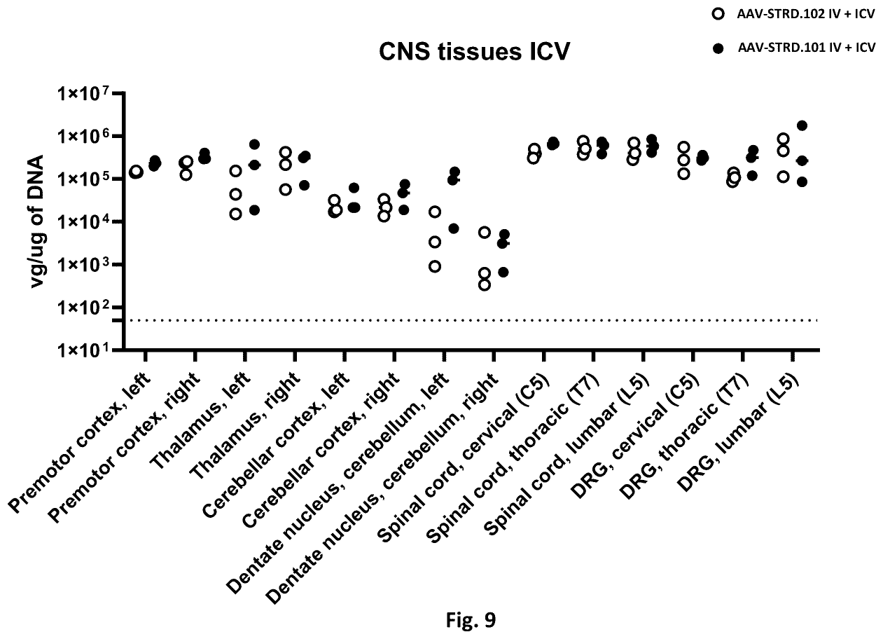

Recombinant AAVs were administered to non-human primates, in order to determine biodistribution. Recombinant AAVs were administered by intravenous (IV) and intracerebrovascular (ICV) injection (FIG. 9). AAV-STRD.101 was administered at a dose of 2.9×10¹³ vg/kg by IV injection, and 2.1×10¹³ vg by ICV injection (black dots). AAV-STRD.102 was administered at a dose of 2.8×10¹³ vg/kg by IV injection, and 3.0×10¹³ vg by ICV injection (white dots). After 30 days, the animals were sacrificed, and viral load in various CNS tissues was measured by qPCR.

As shown in FIG. 9, both AAV-STRD.102 and AAV-STRD.101 infected various CNS tissues. Additionally, because the AAVs showed high levels of transduction, this data suggest that these AAVs are likely to avoid neutralizing AAVs in vivo.

### Example 6: Cell Therapy Method for Treating a Subject in Need Thereof

Cells are transduced using an AAV vector ex vivo. For some purposes, the cells may be autologous (i.e., derived from the subject to be treated) or allogenic (i.e., derived from a different subject/donor). After transduction of the cells using an AAV, and after expression of a transgene has been verified, the cells are administered to the subject using standard clinical methods.

Cells may be administered to the subject once, or administration may be repeated multiple times at therapeutically effective intervals. The number of cells administered varies depending on, for example, the disease or condition to be treated, the severity of the subject's disease/condition, and the subject's height and weight.

### Example 7: Gene Therapy Method for Treating a Subject in Need Thereof

An AAV vector described herein (e.g., an AAV vector comprising a capsid having the sequence of SEQ ID NO: 175 or 180) is administered to a subject in need thereof, wherein the subject has a disease or disorder of the CNS. The AAV vector is administered to the subject once, or administration may be repeated multiple times at therapeutically effective intervals. The administration is by one or more therapeutically effective routes, such as intravenous (IV), intracerebroventricular (ICV), or intrathecal (IT) injection. The dose of AAV vector varies depending on, for example, the disease or condition to be treated, the severity of the subject's disease/condition, and the subject's height and weight. For example, the dose of AAV administered to the subject may be 2.8×10¹³ vg/kg or 2.9×10¹³ vg/kg when the AAV vector is administered by IV injection. When the AAV vector is administered by ICV injection, the dose may be 2.1×10¹³ vg or 3.0×10¹³ vg. In some protocols, the AAV vector may be administered to the subject by both IV and ICV injection.

The foregoing is illustrative of the present invention, and is not to be construed as limiting thereof. The invention is defined by the following claims, with equivalents of the claims to be included therein.

1.-73. (canceled)

74. An adeno-associated virus (AAV) vector comprising an AAV capsid protein, wherein the AAV capsid protein comprises an amino acid sequence of SEQ ID NO: 180.

75. The AAV vector of claim 74, wherein the AAV vector comprises a cargo nucleic acid, wherein the cargo nucleic acid is encapsidated by the AAV capsid protein.

76. The AAV vector of claim 75, wherein the cargo nucleic acid comprises at least one terminal repeat sequence.

77. The AAV vector of claim 76, wherein the cargo nucleic acid comprises a heterologous nucleic acid sequence.

78. The AAV vector of claim 77, wherein the cargo nucleic acid comprises inverted terminal repeat (ITR) sequences located 3′ and 5′ of the heterologous sequence.

79. The AAV vector of claim 77, wherein the heterologous nucleic acid sequence encodes a polypeptide.

80. The AAV vector of claim 79, wherein the polypeptide is a therapeutic polypeptide.

81. The AAV vector of claim 79, wherein the polypeptide is an immunogenic polypeptide.

82. The AAV vector of claim 79, wherein the polypeptide is nuclease.

83. The AAV vector of claim 82, wherein the nuclease is a Cas9 nuclease, a Cas12(a) nuclease (Cpf1), a Cas12b nuclease, a Cas12c nuclease, a TrpB-like nuclease, a Cas13a nuclease (C2c2), a Cas13b nuclease, or a modified or truncated variant of any thereof.

84. The AAV vector of claim 77, wherein the heterologous nucleic acid sequence encodes an untranslated RNA.

85. The AAV vector of claim 84, wherein the untranslated RNA is a guide RNA.

86. The AAV vector of claim 84, wherein the untranslated RNA is an antisense RNA, a ribozyme, or an interfering RNA.

87. A pharmaceutical composition comprising the AAV vector of claim 75 and a pharmaceutically acceptable carrier.

88. A method of introducing a cargo nucleic acid molecule into a cell, comprising contacting the cell with the AAV vector of claim 75.

89. An adeno-associated virus (AAV) capsid protein comprising an amino acid sequence of SEQ ID NO: 180.

90. A nucleic acid comprising a nucleic acid sequence encoding the AAV capsid protein of claim 89.

91. An expression vector comprising a nucleic acid sequence encoding the AAV capsid protein of claim 89.

92. A cell comprising the nucleic acid of claim 90.

93. A method of producing an adeno-associated virus (AAV) vector, the method comprising:
a. culturing a cell that comprises (i) the nucleic acid of claim 90, (ii) a cargo nucleic acid comprising a 5′ inverted terminal repeat (ITR), a heterologous acid sequence, and a 3′ ITR, and (iii) AAV sequences sufficient for replication and encapsidation of nucleic acid, wherein the cell is cultured under conditions such that it produces the AAV vector; and
b. collecting the AAV vector from the cell.
